SEQUENCE LISTING (33 sequences)

SEQ ID NO: 1 - DNA, 1563 bp, Homo sapiens
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaactgggctttcctggtgctgccatcgcttctgatactgctgttagtaattgccgcagggggtgtgatctggaagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 2 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 3 - DNA, 34450 bp, Homo sapiens
  misc_feature (10013)..(10013): Wherein “N” is A, or T, or C, or G.
gaaagagagagaaaaaagaaggaaggaaggaaggaaggaaggaaggaaggaaggaaagaaagaaagaaagaaagaaagaaagaaagaaagaaagaaagaaagagagagaaaggaaggaaggaaggagaaaagaaagtcaacagtcaacatttcagagatcccaagataccaacactgaccgtgcctgctgctcttccatcctcctccaccctgcgcctttgaggtggaattgcgtcctctgtgagcagggctttgttaagagatcctaattaaggccaggcacagtggctcatgcctgtaatcccagcactttgggaggctgaggtcacctgaggtcaggagttcaagaccagcctgcccaacatggtgaaaccccatctctacaaaaattagctgagcatgatggcaggtgcctgtaatcccaactacttgggaggctgaagtgagaaaatagcttgaacccaggaggcggggttgcagtgagccaagatcacactattgcattccagcctgggcgacagagcttttgtctaaaaaaaaaaaaagaaaaaaaatcctgattaagcagaagccttgatgctagtcccagaagcatcctgaaatttccaaaagaaatttcccccgcggttaaactcagagcaacttttggacccaccaagctctgtgaaaatcattttctcttccaaaaactgatgggaccaaagctgatcccagtttcaaataattatcaaaaaattggaaacgaaatatgatcagaaaagaagaaagttgaaaaagaaaatccttatcacccaaagacaacaaccattaatattttggtaattattattacaaatatctttctatgcatacagacagactcacacacacacacacacacacacacacacacttttttttttttttttgaaactgagtttcactctgtcgcccaggctggagtgcagtggcgcgatctcggctcactgcaacctccgcctcctgggttcaagcgattctcctgcctcagcctccctgatagctgggattacaggtgaatgccaccacgcccggctgattttctgtatttttagtagagacggggtttcaccatgttggccaggcttgtctccaactcctgacctcaggcgatccacccgcctcaccctcccaaagtgctgggattacaggcgtgagccaccgcgcccggctacacacacacttttttaatgggcctatgttttagcactcgcttttctgtttctcagtgtgttgcaaacacctcggtgtcgatacacaccattcggcaacgtcctcctaaagggccgcataatattgcgcgtcgtggcgtgtgccttactgggaagctactgctgtccaggtgaacaccacagccttcggggtcagaaagacagctttccccagaacaagcacctgaagctctggggcctgccgctccccgggtagagaagtacgtggagaagggcagcacggatccgccgggatccccgggggcattaaagggaatcgcgtgtgtaaggcgcggagctcagcatccggctcagaaacgcgctcggatcccgccaatggcattgaggccgcgtagccaaaccggccttgaactctccctaatcctgccaaaatggcccgtcctggagcactggactggccgtgggttattgatcatcagccggtttcttcccctcccctgcccttcccccgtgcacggatttactgatttttttttccgggaattgagtaaaacaaaactaagtgcagatgaagcagaggtacgggcgagtttcgagcgcggggaccggcgcgctcccccccccctccccccgcggcggggctgtccccagggaccttctcagtgaatcctaggcggcagggacgggcccgcggctctgcgggccattggctgccgactgcgtcacctgcccgcggtgggctaggagacgggaggcgggaggcgggaggcggggacctgggtccgggcggggacgccgcggcaggaaggccatggcggggcccgagcgctggggccccctgctcctgtgcctgctgcaggccgctccaggtaagggcgcggggccgcgggagggagggggaagagggctccccgggccgggccgcgcctaccctcggacccagagctcctgggacaggcacggggtccgcagccacccgagccgggtgcgaatcggccctgcctacgcgcccccagtttgcttcttcccaggactgaacagaaccgggtctttgatattcctctcccgcaggaaacgaatccagtttcctaatgcttccagcttcaggagaactggagaaaaaagacagcggcagtttgatactgcatattttttaataaagtgctttttaatgtttcctaaagaaagcactgatccctgcgtgaaaaccacacttgaccctaaagtgtggacagcagggaaagtgggaccgattgatgtcccttcccgttcctgccaggcctctggtgggacggagctctggtcgcctgtgccctgctttctaacaagacggctttcttttggtggtggttgttgttttgttgttgttttgttgttgttgttgttgttgttgttttcccacctctactgatgagtaaggtgtcaggtacaaaattcctcgccgtaggacccaaccaccaaacctcaccgcccacgactccaaccgaagcagggaagagaaggtccagaaatcgcccccaggatattttcctagtcttggactcacagtttaaagagctgtaaaggtccctgggcataatccaatcatcataaaagcctatatttattcagcaacttctttgtgccaggcaccgcattattctggaagcctcacgacccagccatcctaggaggtagatattatttttacttttccgatgggaaaactgaggctcagagcaattcagggaattcctcaagaaggacggcagaggtgaggcacacagaagagagaagaggggctaaagcaagcctggctagcttttgcctccagggtaggcacgtgggacaggctgtccatccactgggtcactaggccagccagggatgctccagcccccagtgcccacagcagcgttctctgtggctgatgagggaccgtgtacctgtgtgtggagggagggtggggtcttctgttcccctttcactgtcaagcccagaccttcttgtactttcacctgataagtatttaatatacacaacactaactatggtgtgatgatttaggagtaagtacagccagatctaagttcaaatactggctcccacacaaactgactgtgtagcctcaggcaagttagttagcatctgtctctgagcctagcgccctttccatggaagcagaatgaatgacacctaccccatagggtggtctgtcccaagggtgattgaggttttacatgtaaagagccaaactagtgcctggcatcctttgaaggcttcatagaggaaagttgctctagctgctgtttttctcatgtgacctagctcgaatctggggactgtcctgcccataggataccttacaagtggcttgcagacagcctggtctcctgctggtcacccgttaggaagtccagaagctgggagtagtaatagcactagcctcgtggtgatacagtcccagctagaggacacaggatgaggtggaagcaggcacccacttttgggtctaaaaggtgatgggtaggcagccgaggctggggacagccatccacagaactggaccctccctccctgatgccattttgcaacccgtatggatttccatcatggcacatgggacacttcaggaccctgaattctccatgggaccatgagctcctatagggcaggaatgaagttgtgttcttctttgaaacccctggcacaccgtggtcaacagatcttgtttgactcgtagtggtcaatagatggaatagttggaatcataaagctcaatagaccccatgagaacctagaagacaaagtacagtcaagagctcggactttggagttggctaggcctggactgaatctgattctacaacttaatagctgagagggccttggttttcccatctgtaaagattataattattataatgaatacctacctcctagggatgtaatgaggattaaaagagaaagtgcaggtaaactgtttaacacagaacctggctcatagaacacaatacacattagctgctattattattattattattttatttatttattttgagacagagtctcactctgtcacccaggctggagtgcagtggcgcaatctcggctcactgcaacctccacctatcgggttcaagcaattctcgtgtctcagcctcccaagtagctgagatgacaggcgtgtgccaccatgcccaactaatttttgtatttttagaagagacgtggtttcaccatgttggccaggctggtctcaaactcctgacctcaggtgatttgcctacctctgcctcccaaaatgctgggatcacaggggtgagttaccatgcccggccttagctgctattattatcatcatcgttatcatcatcatcatcacctcgtagatatgtcaaggaagattccctggaggaagtgacatttgaatcaagtatttcaaagactagatggtgaataccaggcagtcaaagacacctgggtttaaaaacatccagaagaatgcagtggcttggcaacatcgagcaggaagattgcctgatgagcctgtagggtagctgttggggagagagcagcaagacggcctggccaggccaggccaggccacgtcaggcagggcctcacaaacctcaataacaaatgtggactttattctgaggccaaggaaagggcatgaaactggggagtggtgtaatcagatgcgtatttcagaagatgaagattaacagtgagaaggaaaatgtgccacagaggggaatagaggtcagttaaagggagtcagggaaagtgtcctcgagacagtgacatcaaaggaatgtgaaaacagcaaaggagtgagccaggtggatatccaggggcagaactgttaaggcagagggaacagcatgagggaacagcgtgtgcaaaggcctggagttgggagtgtggctggggtgctccaggaagggcaaaaagtcctgtgtggatggagatatgggagcaagggaggagtggtgggtcagattgggtagggccttggtggtgattgtaaagactttggagtttagaccaggcacagtggctcaggcctgtaatcccagcactttgagaggccaaggtgggcggatcacctgaggtcaggagttcgagaccagcctgtaatcccagctactctggaggctgaggcaggagaatcgcttgaacccggaaggtggaggttgcagtgagctgagattgtgccactgtactccagcctgggtggcagcataagactctgcctcaaaataaaataaaaataataaagacttttgagtttccctggagtgagaggaaagccttagagggctttagcagaagatgaacatgatctgattttcatttttaatccttccctgctaatgtggagaatggactgaaggcaaggtgttttgtatatttgtctgtttcgtagagacagggtcttgctctgttggccagactgaagtgcagtggcacaatcacggcagccttgaactcctgggctcaggcgaaactcccacctcagcctccttactctcaccattgtgccctgctaattttttaaaaaatttattttgtagagatgtggtctcactatgttgcctaggcaagtcttaaattcctggtctcaaatgattctcctgcctcgatgtcccaaagtgctgggattacaggtgtcagctgccatgcccgacctgtatttttttttttaatggggaaaaagccttttaatagtatgaggtgttttctggtgtttctaccataaagctcttctgtaaatcaaaatgagaatgtaattattgatagagcaatgaccttagactacagtgcagacttttcatcttacatttgggctcatgaattttagtataactgattatgacagtgttttttacatagttatgatctagagcagaactgaaaacaaaataacacatactctacatcaatatattcgttcagtaatatctgggcttggatgaacctgcagaagtaggtaaagctgtcagatattttcttaaaccaacagaaaagaaatgtatatgacagatgttgtgtttacttacttatttatttatttatttatttatttgagatggagtctcactgtgtcaccaggctggagtacagtggtgtgatctctgctcactgcaacctccacctcccggattcaagcgattctcctgcctcagcctcctgagtagctgggattacaggcgtgcaccaccacgcctggctaatttttgtgtttttagtagagacagggtttcaccatgttggtcaggctggtctcgaactcctgacctcgggatctgcccacatcagcctcccaaagtactgggattacaggcatgaaccaccacgcccagcctgtatttatttttttaccactatggagtccaatatgaaattctcacaactatgcatatacattattaacatgtaagcacacctaggtataaatatgcacatagtccattaattacatcaggggaattaaaaacatactttcaagttaaaatgaattttcaggaaaaaaactgcattcacaaatctgaaatgtgaatacaaaaatgaaattgtgaaataaataatgaatataggtgtcacctaaacttccatagtaacatgcctccaaatgtggatttagtgatcatccaccttgggacaagggcttttgagagcctccagctaaattagggttccagtagcagagtggctggcaagcctgccctaatgaataatgccagcgagctgggcgtgggtacttacagtgtgcccttcatggaatacttttttttttttttttggaatggagtctcgccctgttgcccaggctggaatgcagtggcacaatctcagctcactgcaacctcgtcctcctgggttcaagcaattctcgtgcctcagcctcccaggtagctgagactacagccctgtgccatcatgttctgctaatttttgcatttttagtagagacgaggtttcaccaagttggccaagactggtcttgaattcctgacctcaggtgatctgcccaccttgacctcccaaagtgctgggattacaggcttgagccactgcgcccggcccatgaaatacttcttacctggcggacagcctaatagcctagctgtctaacccatggctgggggtccttcacacttgtttatactggcagacgtccctgtgactcttgtctgatccatgtccaagtttatgcctgtctgaccattgctctggcgctgggagccagactgtgttcccagcaacccagggaaaaccaggcctgggctgggcctgggttcctgagatggaaggtgcaaattcagtacaccacctcaatgcaaaacaagttcaaaggcttattacttacagatcctgagcagggaaggtgcaatgagtagggagggtcatcctccatcctgggctacatgaagcgggaatgaagagtcaggcaaaaagaaagtgagagcttgtggcaatgagaagtatattatgtaagggactagggtgtgggtcaggttaagtttgagggcaaatgcttgaatgatccctttaaaggaatgggtgggaagtggggagcccagtttgccgggagggagagatgcctcgaagttcttatctctggccactggcttgggccatctgagtgtggcatctacttctaatgcctaggcagcaacctttgctgtgtcatctcccttacacaaggttggaagcagggagaccggtcaggaagcctttggtgtaacccatgttattgtaatattcattcatttactcaacagatgtttattgtgcacctactatgtgctgaggccatggcaggcaggctctggggatgtggctgagaacaggacagagcccctggtccttgatatcctcaaggatgctccctcctggaggccattaggttcctgttccatggtgttctgctggaaccctccggtcccagagtgtgcaggagcctcccctcctggcaaagggtcttctctcatggcacaagggctgcagtacagccagtcagtggctcctggttcctcaaactcagtgagcacttgcctgcccttcgtgctgcccctcagcttgggatggcctgagtcaagaccagccaggagctccaggcttcatgacccctttctttcccccagggaggccccgtctggcccctccccagaatgtgacgctgctctcccagaacttcagcgtgtacctgacatggctcccagggcttggcaacccccaggatgtgacctattttgtggcctatcagaggtagaggagactctctcggctggtggatgggaagactgagggggtgggtgggggcttggaggggcttctctgggacagctgcacccagtgtgggcagcactggctagctctctgggccctacgggagatggcatgtggccggcatttggagaggggcttttgataaaggtctggaggtggggaagatgttgaatgaagagcagtgtacaggtgaccagtctgccggggcgggggtaagtctttgaggaaagttggtgtggggcatggatgtagctgtgggggccagaggatgaaattctcaagtggctggatgaggtgcttggagctgtcccagctgatcagtgaggcaactaggtacacggcagaggagctgttacctgggcaattaggcatccctcaatgatcacactttttttctcttttttttttttttttgagacagagtcttggtctgtcacccaagctggagtgcagtggcttgatctcggctcactgcaacctccacctcctgggttcaagtgattctcctgcctcagcctccagagtagctgggattacaggcatatgccaccacatctggctaatttttgtatttttaatacagacgaggtttctccatgttgcccacgctggtctcgaactcctgagctcaggtgatccacccacctcagcctcccaaagtgttgggattacaggcgtaagccaccgcgcttggccaaatggtcacacttttcccgatgggatcattctcaatttggaagcccaggcagccacagcgaatccagagaaatctgacaatggaagcagatccaccatcttcgaacatagatgggaatcgttcagagttctttagcaggacagtgagatgatagaagcagaagctcgggaggattcacctggagttggtgaggaggggaaagcaggaagaggaggggacccaccgtgtcctcaggacccgtcctgtgccaggccaagtgctaagggccctacgtgaatatttcacttccttctcccaatgtgaccaggcaggctctgtgttttccccattctagaggtgagggggattgagcactgtgtcaacacatgtaatgaacttaatctcacagcagctctctgaggacaagttcagtacgcctctttacagaggaggagactgaagcaccaagggtgcatgttgctcaaagtcacacagctgggcgtagtatggctggaataaatttattaaggagttgaaagtctatcctctaggaccaagcatggtggcttacatctgtaatcccagcactttgggaggccgaggtgggtggggagattgcttgagtccaagagttccacaccatcctgggtaacatggtgaaaccctgtctctacaaaaaaaaaaaatacaaaaaattagtgaagtgtagtagcatgtgcctgtgttcccagctacttgggaggctgaggtgggaaggatcacttgagcccaggagatggaggttgcagtaacaaagatcacaccactgcactccaacataacaacagagcaagatcaaaagggtttttagctcccactgaacgccncgtcatanccttaggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaacaacagagcaagatcctaaaaagaaagaaagtctatcctctgaacttctatgatatttttcatgtcttttatacattagaatggtgatattctaattatataatttttttcatttgttagttggaattattttataaagagatgtatcctctcatctggtatttgatatccagtcatactattcaaataggcaagagaggataaatgcttaatttttttcctttatcaattttcaagataatgaattggttccttatcatctcccaaaggtgattgctagtttattattatcattatgaactcaggcatttaaacacatttggtggtttcagtctattgcgacgtactctgctcattgaagcttgaattgcctcatctctgtccagtgggagtctcatcaagtttgctcctgagtccttttaacttgaccctagtggtcaagttaaatctttccagatttaacagatacctttccagctgtccattacgacaagatgttccaggtccctctggtacaattcctgacctaaaacctgcagtcagccatttctccatttagtaagaaatggttataaagactataatctgcatgctagctatgctgatcactacttagctattgcttttggtgttttcagtgaacagagtgatgtgtgtataccacatagacacacacatgtacatactttttttttttagacagagcttcactctgtcacccaggccagagtgcagtggcatgatctcggctcactgcaacctccacctcctgggttcaagagattatcctgcctcagcctactaagtagttgggattacaggcgcccaccaccatacccggctaatttttgtatttttagtagagacggggtttcaccatgttggccaggctggtgtcgaactcctgacctcaagtgatctgcccccctcggcctcccaaaatgctgggattacaggcatgagccatcgcacccagcctacatgtacataatttttaagataaaatgcctaatgagttatacgggtgcttcccatctaaatttagttccttaggatttttacctgacttctatggtacatctatattttctttctttcacactgagaatcctgtttctcaaggacaggggacatgatagaactagaatgacccataattactcattttctttatcccaaaacatacatacttgcctcttaatagtttcttgctcttttcgcccaaagggtttgtgatggtcaatattaggtgtcaacttaattgggttgaaggatgcctagatggctgttaaagttttgtttctgggggtgtctgtgagggtgttgccagaggagactgacatttgagtcagtggactgggaatggaagactcgtcctcactcagtgtgggtgggcacaacccaactggctgccaggctggctggaaagcaggtggcagatggtgggatagcttcacttgctgggtcttccagcttccttctttctcccgtgcgggatgcttccttctgctcctcctgcccttgaacatcacactccgggttttttggcctttagactcttggacttaagttagtggtttgctgggggctctcggatctttggtcacagactgaaggctgcactttcagcttccctggttttgagggtttcagattcggactgagtcactatggcttctttctttcccaccttgctgacggcctatcgtgggacttcgccttgtgatcgtgtgagccaattctccttaataaactccctttcatatatacgtataacctattagttctgttcctctggagaaccctgactaataaagggttgttgctttttctttaaaatctagtaattttatttgactgtgtgttggtattgctcattcattctgagttgatatttttaggcactcaatattctcacttaatacatggttccaaggcatttttattttaggaaggttttcttaaattatagttttagtatttgttctattctcttgttttgattttcttctttagggactcatatcacttgtatgttggatcttctttttctgtgttcagtatttgtcttttgggcacagagactcacacctataattccaagactttgtgaggcataggtaggaggatcgcttgagcccaggagtttgagaccagcctgggcaacatggtgaggccctgtctcaaattaaagaaaaaggagagaatacttgtctttttctttcaaatgccttttatctgtctgtctatctactattctgctctctaaatgaaataggtttcactcttgagtttttaaaaaactgtgtgcttccatgtgtgagattattcaacatcttatttgtaatctttctcttggttacatttatttttcctgaaaactctagtctgcttttagctgacatgtttgtagctaagagcgcacatttcttatcatagcttgccgtgctgaattaattccaattttcttttaaaaccaacattattgagttaaaatgtatatagaataaactgttcccattttaaagtatacaatttgatgagttttgacaaaagtgggcacccacgtacccaccaccacaatcaagatgtaagacgttctctatcaccccagaaagttccctcatccactttgcattcaggcctccagatctaggcaaccacagatctgctttctgacactgtggattaaactttgcctgttccagaatttcatataaatggatgtgtatagtatgtaccctttcgtgtctggctcctttccctcagcataatgtttctgaaattcacccacattgttacatgtatcagtagttaattcctttttattgctgagtagtaatgccattgtatgactatgtatgacatttgttaatccattttcccgtcagtggatatttgggttgcttccagttctgggcaggtattcatttgctagggctgccatatgcttgccctctggcctcccaaaatttgtgtccttttcatatgcaaaatacattcaccccctcccaacagccccaaaactctcttttttttttttttttgaaacagagttttgctcttgttgcccaagctggagtgcaatggtgtgatctcggctcactgcaacctctgcctcccgggttcaagagattctcctgcctcagcctcctgagtagctgggattacaggcatgcgccaccacgcctggctaattttttatatttttagtagaaatggggtttcaccgtgttagccaggctggtcttgaactcctgacctcaggtgatccgcctgccttggcctcccaaagggctgggattacaggcatgagctactgcacctggctagccccaaaactcttaacccatttcagcatctactctaagtccaaagtctcatctaaatcaggtatgggtgtgactggaggtgttactcatcctgaggccaaattcctctccacttatgaacctgtgaaaccagacaggttatgtgctttgaaaataaagtgatgggacatgcatgggatagactttcccattccaaaagagaaaaataggaaagaaggaaagagtgacaggtcccaagcaagtctaaaacctcgcagggcaaattccattagattttaagtttcaagaatagccctctttggctcagtgctctgccctttgggcccactggggcggcagccctatcccctttgccctgggtggtgaccctaccctcgagtcactggttagcagcagcctagcctgctgaaactaaggaggggacagtgttgcctccaggtctttggtggcagtgacaaccctgctgatctctgaatcatcttccaggaaatttttccctatacttgaaggatattgcgtgttcacagccaaatagctccagctcttgtccctttctttagaatcccagaagtccaacagccttccttcattctgtcccatctctgtcccctttagtcaaagctggaagtgcctctgctggtataatcccatcagtatgtctaatttctgcttaaatggctgattaagtctatgagttgcacctctgatctctttatcaaaaggttgttctagccacaaccttagtgtcctccccagaacatgctttctcatttttttttttgcaatgtggataggctgaaaattttccaaagcttcaagttctagttccttttggcttaccaattcttttcatatatctcttctctcacattttactataagcagtaagaagaaaccaggttgtaccttcagcactttgcttagaaatctcttctgctaagcatccaagtttatgtcttttaaattatctttttgttatttattttatattatcatttttgagatggctagccaatgatcttttaacttctaatttctgcaaaacactagaagacaattcaaccagttctttgccactttataacaaggatcacctttcctccagtttccaataacacattcctcttttccacctgagacctcaccagaatcacctttaatgtctatattcctaccaatagtctttttaaggcaatataggctttctctaacatgcacttcaaacttcaagattctacccattatgcaattccaaagccacttccacatttttaggtattgattacctcagcacctcatttctggtgcccaaatctgcactggtttgctagggctgccataacaaagtacgacagtctgggtaaacaacagaattttattttctcaaaattctggaggttggaagtccaaggtcaaggcgttgctaggtttagtttctcctgaagcctctctccttggctagcagatggctgccttcttgctgtgtcctcacgtggctttttctctgtgtgtgttcactctggtatctcttcctcttcttacaagtacaccagtcctactggattagggccccagccttattacttcatttaaccataattacctctttaaagctcttatctcaaaacacaataccactggggatgaggtcttcaacatatgaattttgggggaactcaattcgtccataatagggctattatgaattaagctgctgtgaacattcatgtacaagtctttgtgtggatatgttttcatttctcttagataaagatctaggagtatcagcctgggcaacatagtgagaccccatctttacaaaaaattttcaaaattagccaggcatggtggcgtacacctgtagccctgccatctcaggaggctgaggtgggaggatcccttgagcccaggggttttagactgcagtgaactatgattgcaccactgcaccccagcctgggtgacagagtgagactctgtctctaaaaaaaagagagagaggggaggaaggaaagaagaaagagagggagggaaggagggagggagggagggagaagaaaaatggatctagggttaagatttaggagattaggtaatgaatgtgtactattacagggaactgtcgagctgtttccaaagtgactgtaccattgttcattgccaccaacaatacatgagagttctagttactccatgtgcttgttacacttagtattatcagtctttttcattttaaccattctagtgagtatgtagtagtattttattatggctttaatttacaactccctaatgatgaatgatgttgaacatcttttcatgtgcttattggccattcatatatcttttgtgaagtgactattcaaatatttttccactttttattaggtcatttattttcttattattgagttatctatgaatacaaatcctttatcagtgtatgtattgtgatttttttccccagtggctggccttttcattttcgttaggcttttttggtgggtttttttttttttttttggaagagaaaaatattttaatttgataaaatccagtatatcaggtgttatagactgaattatactctaccccacaaattcatatgttgaagccctaacctctaagtgactatttggagatgagcctttaaggaggtaattaaagtaaaatgagatcataagggtgggccctaatctaataggactggtgtctttataagaagaggaagacaccaagagcgcatgcacacagaagaacggccttgtgaggacacagcaagatgacggccatctgcaagccaaggagagaggcctcagtagaaaccaaacctgctgatgccttgatcttggacttccagcctccagatttctgttgctgaagccaccctgcctgtggtgtcttaccatggcagccctcacagactaatatatcagatttttttccttcaacagttaacgcttttggtgtcctaagcaatattcgcctgacccagggtcatgaagatttttcttctatgctttcttctggaagttctataattttagcttttacatatttttttaactttccttcttcttgccttctgtttcttttaaggcatcatctattgtgttaatttgttcttgtattccttctgatttattcttcacttctgaaatgaattttgctttttaaaaatatatataattcttttctgtgtctgagtttttctaattaggttttatgtggttttttcttgtcctgcatcactttttactgtcttttgcccattttgaagtatcaggttccagttttgatctgttcatggatatgtttttgtgacatgtttcttctggcttcttatcatttattgcttagcttattaatttctattctttcttattttctattataagtatttaaagctatatgttttcctctaagtattacttagctgtcttatacgttttcatttgtgttatttggtgatcattcactttcagctatttattaatttccattataattctttcatctatgggttgttttaaaaaatatttttaaggccaggtgtggtgactcacatctgtaatcacagcacttagggaggctgaggtgggaggattgcttgaggccagaagtttgagaccggcctaggcaacaaagtgagaccccctctctacagaatatttttttaaaattagctgggccaggcgtggtggctcatcccagcacctgtaataccagcactttgggaggccaaggcagatggatcacctgaggtcaggagttcgagaccagcctgggcaacatggtaaaaccccatctctactaaaatataaaaattagccaggtgtggtgataggtgcctgtaatcccagctacttgggaggctgaggcaggagaattctttgaacccaggaggaggagtttgcagtgagccgagattgcaccactgcactccagcctggatgacagagcgagactctgtctcaaaaaaaaaaagaaaagaaaattagctgggtgtagtggcaggtacctgtggtcccagtgactcagagactgaggcaggaggatcacctgagcccaggagtagaggctgcagtgagctatgtttgtgccactgcactccagcctgtgcaacagagcaagacgctgtctcaaaaaatatatatttttttaaattttcaaacttcctttagttctctttttgttattaacttttaactgaatgttttgcaatcagaagaaatactttatgagatacctattctttaaaatttcttaagaattgctttgtgttaatattttgttaatagttcacatgtggttcaaccaatttgtttagttagttctgtatatgttcattagaccaacttgataactgtgttgttctttatttatttatgtatttatttttctttgtctattcatcaattgctgggtgagatgtattaaaatttcttgttgtaagtgtggctgttcactttctacctgtagtttgtctgtttgctttatagagggtgaagttgtttagtaggcacacataagttagaatttttctgtcttcctggtgaatggaatcatttatcattatctaatgttcttttcatctttagtattgctttggacttggaagtctgtattttgtctcctgttaatataactacactggttcctttggtgtgaatatttgcatagtataacattttccatgaagaaacaaaacagaggaattggttctttctcaaaatctgatctttgtgtcagcccccatctcagccttctccattcatccttggtcactccccaaacccaggagcaatccttgattctccttttccccacattctacatccaatccgttagcaagttctattagttctattattacctccaaaatagatattgaatccagccctttctcactgtctccaccatcatcctgtctcacatccctaccatggcctccttgctggttgaccagagtgatcttgtaaaaacatgttaggccaggcacggtggctcctgcctgtaatcccaacactttgggaggccaagcgggtgggtcacctgaggtcaggagttggagaccagcctggccgacatggtgaaaccctgtctctactaaaaatacaaaattagccaggtgtggttatgctggcctgtaatcccatctactcgggaggctgaggcaggagaatcacttgaacccaggaggcggaggttgcagtgagccaagatcatgccactgcaccccagcctgggcaacagaacaagactccatctcaaaaaataaaaattaaaataaaatgttaggctccctgggtctctggcttagtccatttgtactgctttaacaaaataccttagaatggtgtaattctaataattgctattaataaataatagcaattaataaataatagcaatttccttctcacagttctagaggctgggaagttcagggtcaaggtggcacctgactccgttctggtaagggcggctctctgcttccaagatggtgccttctcgctgcgtcttcgcatagcggaagggcaaacactgtgtcctcacgtggcagaagagatagaagggccaggcagctctctgaagtatccaggttggagtcatggacctgcatgttcccctctgacatccacagagtacctatcatggtccttggcatgcagcaggtggcccataaacgcctgaatgaacaaacatatagtaatggtcgctagtactaggaatagcagccaccgcaacagtcctgtgagggaggcattacagatgaggaaactgaggtttaggggcaaggacctgcccatggtcccaaagctagggagggacagggctgggattcccactcccatccatctggctccagaacctgagctcctgaccaggctgttcttatcctgtctcagccagtggctgcctgtctggacggatggacctaaagtcagtccagccaaacagagggaagcatgatcaactgttctctaagttccctgacccggagaggctgagtccatggcccaagctctcctctctcctcccccagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaaggtaggtctgtgggtaagggactgagtggaaggctgtccatcccatcggggagctgtgctcagtgctcagtggttctgttctcctgaccatctgtctcccacttccccaaagcagagggcagctccctgggccaggccctttgagatggggtgtgggaccagcaacagcgagggaccatgtctggtagcctgtcagggagttaggggagctccagccagcaccagcaatctcacgtgcaccctctgctaacaatgttcattattttcagttgagcaccattttggtcatggactacacaaggcactttatatgcttattcctatttttttatgttcagcttctctccttaaaaacaatgtttaaaaccaattctgggccaggcgtggtggctcacgcctgtaatcccagcactttgggaggccaaggcaggtggatcacctgaggtcaggagtttgagaccaccctggccaacatggcaaaaccccgtctttactaaaaatacaaaaattagccaggcttggtggcaggcacctgtaatcccagctactcgggaggctgaggcaggagaatcgcttgaacccaggaggcggaggttgcagtgagccaagatcacgcccctgcactccagcctgggcgacagagcgtctcaaaagaaaaaaattaataaacaaagaaaaaaaaacaaattctgtttgcaaaagtattttctatacactgtagaaatttgtggggtgtgggggggtaaagatgatagaaaaaaaaatgtcccatgcttactggcagaaatcatgtattgacattgggtgaggagggcactttttttttttcagtctatttttaatcttcacagcaaacttgtgaggttcatttccatcaacctgagactcacagaagctaagaaacttgataccgctagtaaccagtggacttgataccgctagtaaccggtggacatagatgtgaactggatctttctgacctcgggcagggccgggtaacaaggggaggataaatgcccagacagtgtcctcagagagctgagagctgtaacttgctgcccgggcttctcacagtgttcaaggacaaaataaggctttaagagagaagagggacagactgattgcagggcagcaggaagagatggtagagaaggaagaagagatgattcgtgtggaaagaagctggctcggtggatggataaaagaagggaaggacagatgggtaagaagaaagggaggatggaggggatggaggaggaagcaatggaaaaatgggaaggaaggaggttggatggaaggatagatgcctattaggaaggaaatatgtgtggatagagagatggaggataggaagtatgttagtcaaggttctccagagaaactgaaccaataggatatatacagatacactaagaggaggccagccgggcgcggtggctcaagcttgtaatcccagcactttaggaggccgaggcgggcggatcacgaggtcaggagatcaagaccatcctggctaacacagtgaaaccccgactctactaaaaatacaaaaaaaaattagttgggcgtgatgatgtgcgcctgtagtcccagctgctggggaggctaaggcaggaggatggcgtgaacccaggaggcagagcttgcagtgagctgagatcgtgccactgcacttcagcctgggtgacagagcaagactccgtctcaaaataaataaataaataaataaaaagaggccagccatggtggctcacacctgtaatctgagcactttgggaggccgaggcggatggatcatttgagatcaggagttcaagaccagcctggccaacatggtgaaaccctgtctctactaaaaatacaaaagttacccgtgtgtggtggcacacacctgtagtcccagctactcaggaggctgaggcaggagaattgcttgaacttgggaagcagaggttgcagtgagctgagatcacgacactgcactccagcctgggtgacagagcaagactttgtctcaaaaaaaaaaaatttataataagaggagatttattatgggaattggctcatgcaatcacagacacaaaaatgtcccccagcatgcagtcatgggctggacaaccaggaaagcttgtggtgtgattctgtctgagtctgaaggcccaaggccaggggagcagtggtgtaacccccagtccgaggccacaggcccgacaatcagaggggccactgatataagtcccagagtccaaatgccggagaacaggaagctccaacgtccaaggacaggagaagttgatgtgccagctcaggaagagagaatgtgaatgtgccattcctcctccattttttgttctctttgggccgtcagtggattggatgatgcctgcccacactggtgaggacagatcatcaccaaatctgccgattaaaatgttaatctcttctggaaaaatcctcacagatgggcccagaaataatgttttactgtctacctgggtatcccttagtgcagctaaattgacacataaacttaaccatcacaggccaggcactgtggctcacacctgtaatcccatcactttgggaggccaaggtgggaagatcctttgaggatgaggtaggcagatcacttgagcctaggagttcaagaccagcctaggcaacatagggagacctcgtctctacaaaaaaaaaaaaaatttaaattcgctgggtacggtggtgggcacctgtggtcccagctatctgggaggccaaggtaggaggatgacttgagcccaggaggtcaaggctgcagtgagccatgattgttccattgaattccagcctcggtgacagagcaacaccctgtcttaaagaaagaaaaaatttaaccatcacagaaggcagaagaaaaggcagatgggtggatgagatgggtgggtagatagtatagaagaaaagcgggacatccaggcagggaaggaagggctggagcgaaggagaagcaaggaaggaaggaaggagagacaagaaggaaggatgtgtagaaaggtggaagagaaaagaagaatggatgtatgggaagaatggatgagtaggttagaaggctcactggctagataaaaggtgagaagtataaatgaataataagaaaggaggcataggaagaaaaaaatattggttagaaaggatgattgagaagaaagggtggttgggaaggaaggaaggaaggatggatggatggatggatggatgggaaggaaaggaaggataagaaggcagacaggaaggctctctggctagaagaatggcagacaaaccacaataattgctgaatgggtaggaataagacattagaagaataaagggaaagacacaaagatatttaaaatgttttcattaattttttgcctcctccctgaatttctcctgattcttcagccccacatcccaagccagggtgatccttcctgcctttacactccctccacactttttctgctctcatatgtggccgtggtcactttcttttggtagtttgcatatttcatttaccccaaactttcagctcctgaaggtcaggatacaaggaggcctcatctccgcattcccctcagctcccttcctgaagcttgatacctagtcagtacccagtggatgtttcctaaacatgtaagtaatgacatcatgaagaagccacatgtttaccttgaccacaaacacagggcaaaggtgactagtgtggtcagagatccctgctggctgggaatcagggaaggctgcatggaagaagtggcattttagttagaacttgaaaggtggtgtatttagttttctctggctgccatattccttgtcacattgccctctccatcttcaagccactgggcaaggctagaaggccctcaacagactatcggtaggaatgtggaagttgaagactcagagtgcagaaagaaacaagtagcattttagagaaaagctaaatcccctccaagaatacctcaatcatcgtgaagagcctgttagtagacgcactaacactcaaggcactgcttcacaaggtaaggaacgtgtaattgaaaacttgagaaaggaagaaacttgttctgtactggcagaaagcttagcagaattgtgtcctgcagtcatatgggacacagagcttgtaaatgatgaatttgaatgcttatccgagaaggtttccaaataaaatgtggaaggcacggcctggtttcttcctgcctcttatagtaaaatgcaagaggagagagagaaaatgagggaagaacttaaacagaaaggaaccaggacttgatgatttgggaggttctcaacctatgcaaaaaacaataaaattaagagattgtagctgggcacagtggctcatgcctgtaatcccagcactttgagagtccgaggcgagcagatcacctgaggtcaggagtttgagaccagcctggccaatgtggggaaactccgtctctactaaaaatacaaaaattagctgggtgtggtggcgggcacctgtaatcccagctactcaggaggctgaggtgggaggatcacttgaacccaagaggcggaggttgcagtgagccaagatcatgccactgcactccagcctgggtgggtgacagagcaagactccatctcaaaaaaaaaaaaaaaaagagattgctcccaaaagtgtgacatagagaaacagccaagtatgtgattataccaaacttcaggaagataaaagatcaaagtactcagtcgctcaaaaggctctttgaagagattaagattataactcacagtccccttcaatcaaaccaggggacttctaggaagctgaacagcattgtccctcagccatatcagctggagccaaaagtagagaagggcttatctgaaaaaaggatctgtggacctggcttttatctaataatgcagtggattcccccatgacatccataggagacccgtaaagttcctgagacgtttacatccacagaaacactgttagcttggattaaatggaacacagagagtatgaaatcaaagaaggctgttggactctccagtttctactgttgagatgcagactggtaaaactacttagctgcaaacacctgctacctttagtgaaaaggaaggatatctcagacggtgaaaccagaagctcaaagggcagtgctaagagcgaaagagaattcttcccaggccttgaaacctaatggagttttcttggctggattttcaaactgcattggaccatgacctgattgtccctttcatgtccccatgcttgagccagattgtctgcaactgttatcctgtgcctgtcccacattttatgttgggagcagaaaactttagttttgctggcccacagatagagagaaactgtaccccgagagttgtactgactggactatgcccagagtctatttgactctgacttagatactgttgatttgggaatttgagttgatgctgtaatgagatgagactttgggggacattgggatggagtgaatggattttgcatttgaaagagatgtgggttgggtaatcctagcccacacctgtaatcccagcactttgggaggccgaggcaggcagatcacctgaggtcggcagttcgagaccagcctgaccaccatggagaaaccccatctctactaaaaatacaaaattagccaagcatggtagcacatgcctataatcccagctactcgggaggctgaggcagtagaatcgcttgaacccgggaggcagaggttgcggtgagccgagatcacgccattgcactccagcctgggcaacaagagtgaaactccatctaaaaaaaaaaaaaaagaaagaaagagatgtggattttgggtgggggacagagggaagaccatggtaggcagaatgatcctctaaaggtgctctgccctaatccccagaagctaagaatatgttagatgtcagtattgcgtggcagtaggaatcttaattaacgttatagactgttatggtttgaatgtcccctctaaaactcctgttgacatttaatcatcattgtgattgcattaagaagtggccctgttaaaaggtgatttagtccttaagaacgctgcccccgtgaatagattaaggtcagtcttgcgggagtgtgtttatcaagaatggattgttaaaaagtgagttctggccaggggcagtggcttatgccactcagcactttgcggggccaagacttgaagtcagttgtttgagaccagcctggccaacatggtgaaagtctgtctctactaaaaaatacaaaaagtgtccgggagtggtggcgggcgcctgtaatcccagctgctcaggaggccgaagcaggaggatcgcatgaatccgggaggcagaggttgcagtgagctgagatcgccccgttgcactccagcctgggtgatagagcaagactctgtctcaaaaaaannnnnnnnnnnnnnnnnnnnnnnnnnnnnnnnnnnnnnnnnnnnnnnnnnaaagaaagaaagaaaagaaaagaaaagtgagttctgccctctcttgctggcttactctcaccctctcttgcccttccacctgccaccatgggatgacacagcacaaaggccctcaccagatgccagtgccatgctcttggacttccaagtctccagaaacatgagccaaatacacttctgttcattataaattacccagcctgtgatattctgtaataacaacacaaaatagactgagacatagatcttcaaatagtgaggttatcctggataatccagatgggcccaatctaatcccatgagcctttaaaactttctccagatggaggcagaagagaagtggcagaaggggaagtcagagagatttgaagcataaacaggactccatggtgccgtttctggtttgacgatggagtggtaacgtgatgaaaaatgtgggtgccttccggagctgagaggctcccactaacaatcggccaggaaacagggaccacagccctacagccacaaagaactaagttttgctgacaacccaagggggcttggaagtgtcttctcccccatcggttccagatgtgagacccagagcgaaggaaccagctgagcccacctggacttctgacctagagaactgtgagataataagtttgtatcatttttaaggcactgtgtgtgtggtaatttgttatgacagcaatagaaaatgaatccagatgggcaggatctgccaggccagtgacatgtggagggcacccaggcggatgggatggcatgagagaaggcaggtcagcaatgagcttgcccaggtcacctctcctctctaagcctcagttttcctctctatgaaatgagagtagtgatatctccctcccagggtcagtgcaaggctgaaataacagattataaggtgctaggtgcacaagaagtgtttgaaacatgctagttgcttttccatttccaagagagctctctggtcttgggggatggaggcagtgcggcccctcgggattactgacaggtcctgctctgtttctgcagtggagccggccccacctgtcctggtgctcacccagacggaggagatcctgagtgccaatgccacgtaccagctgcccccctgcatgcccccactggatctgaagtatgaggtggcattctggaaggagggggccggaaacaaggtgggaagctcctttcctgcccccaggctaggcccgctcctccaccccttcttactcaggttcttctcaccctcccagcctgctcctgcacccctcctccaggaagtcttccctgtacactcctgacttctggcagtcagccctaataaaatctgatcaaagtatgatgacctacaggaggcctgcttgccaagtcaacagattcagtacagaaaaactgaaaaatacagataagctctaagaagcagaccaaaagtacccagagatgaccgcacatcactctggtgtatatccaatttcagatttgttttctgtgtatgcatgtgtgtatagctgcatttatttatggcaagggctggcagactttcccgaagaaggccagatagtcgatatgtttggcttcatgggccgtatgttcgctcaggactactcaacgctgcagttatagcacaaaaggagccgtagcctatacgtaaatgaatgggcatcgctgggttccagtaaaactgtttacaggccaggtgcggtggctcatgcctgtaatctcagtactttgggaggccgaggtggtgggaggattaccttagcccaggagttcaagaccagcctggggaacatggtgaaacattatccctacaaaaaaaaaaaaagctgggtgtggtgatgcatgcttgtggtcccagctgcttgggatgctgaggcaggaggatcgctcgagcccaggaagcaaggccacagtgagccatgatcgcaccactgcactttagtctgggcaacagagtgagaccttgtctcaaaaaaaacaaaaaataaaactttttacataaacaagtggccaaccagacttggtccctgggcctctgctcttgaatgttcttgcttccactaaagtaacattcacactcccgatttttgcatactctgggttctggggaatatagatccgaatccagcgtggttcctgccttcaagaacctcacaaatattctagaccagcactgcccaatagaaagaaatataatgcaagccacatgtgcagttttaagtgttccatgttaaattaagtaaaaagagacgggtaaatcgaattttaataacagattttacttcatccaattgaatggtatcatttcaatgagcaattctgatagtgattgagatcttttacattctttttcactacgtctttaaaatctgatgtgtgttttgtacttggaacacttctcagtgtggaccagatgcatttcacatactcagtagtcacgcgtggccagtgccttccataccacacagtgcagcatctgtagaggtttcctccactgctgatagactaggagaccccaagatggaaagcctgaagaatctgctccttgaagtagggaccttaatggggtgcacgccagggcgaccccaagtggtaggctgcttttgaaccatggctatccctacctctagactcagctgaaaagaactcaggtagtcttgggaagtgcttcctcaatgcttaaactttaatgcaggaaaagaatagaaagttcaggcaaggagggaggatcacttgaggctgggagttcgagaccagcctgggcaacagcaagaccttgcctatacaaaaaataattttaaaaaattacccaggtatggtggtgtggatctgtagtccctagttacttggagagctgaggtaggaggatcgcttgagcccaggagtttgaggctgcagtgagctgtgatcacaccactgcactttggcctgggtgacagaaccaaaccctatcccctacaaaaaaacaaaaaaaaaaaacaaaaaaaaacaccctaccatgtctgccaaccccactctgtcctggctgtgtgaaaccagtccccacagcagctctgccactctctgcttcttttccaaacagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccaggtgggtatcaagtggtgcagaaggagaaactttccctctgggccttgggagcttcgtgacacagtggttaagaacatgagcctagagatagactcgcctggattaaaaccacactcattgtgtgtctttgggcagcttacataatgccccgaaccttggtttgcacagtctgcaggatgggtttattcttgtgaggattaaatagggtcatgtatgtgaagcactcggcacaggtgcagttgtagacaagagccattgttgtttctctcattgttatttttccttccttagaagccaactgggctttcctggtgctgccatcgcttctgatactgctgttagtaattgccgcagggggtgtgatctggaagaccctcatggggaacccctggtttcagcgggcaaagatgccacgggccctggtatagcaaatctgggggtgtgcggcaggtggggaggggttgagagtaagggagtggggctggagctatgagttgttcagatagaatatcaagatggtccagactcttggaccaaaacatctatctttgtgtctgaatttccaccattagtaatgcattcatttagtcctgaataaaatggcaaacaggccctggagggagcagtgccttaagttcctttgagataaataacttcacctctgctaaggatgtgtcagctgctgagagcagagcccctggccttggacctcaggagagacactcaaaaggggaggagaggaggcaccaaaggggacatcttaaaagagttccaatttttagttcacactttaacccaggataagctgtgtcctggctgaccttggagtttcttccctggtctgctgggtctctcccttagaacctaggggcgagctggggcaggggaagcccaggaggtgatataggtcggccctgttcagatgagggctggcaggggcagcttgggcatatgcgaggctccgatgggcatgggggctttgaggatggattctgagtgtccctgcatcgtggcagggtggcaaagggagcatttccaaatttcctggctccaggatctgtgggagaatcccactaactgtcagggtgacaacctcgggtagacatgtctgtgccctgccccgtgccctcagccttcctgttaagagcacaccagctggatttgcaactcccagcgcctgcacccaatgggctttctctggcctctggagcccacattgcccctgcatgtggcaggctgcaagtgtcacagccaccagctcttccattcctcaacaatgactgtgggtaaatagcccaggagcgtccccctcctgggatggttctgaggtgcgtgtgcccagtggctccctgagttgccagcaggattaagtgccagtagccctagtggtcagctgcttgataacaccctgcttcctggctgctcccccagtcccatctggtgtgttctgggatcatctcccaaagaaactgcttacacttgaagccttgtctgaggtctgtttctaggggaattcagatgacgataattatgcttcaggaaagcctaaattttctgcttttctctcccctacccaaatcaggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtgagctgtcccccgacatcccaccgaatctgatgctgctgctgcctttgcaaggactactgggcttcccaagaaactcaagagcctccgtacctcccctgggcggcggaggggcattgcacttccgggaagcccacctagcggctgtttgcctgtcgggctgagcaataagatgcccctccctcctgtgacccgccctctttaggctgagctataagaggggtggacacagggtgggctgaggtcagaggttggtggggtgtcatcacccccattgtccctagggtgacaggccagggggaaaaattatccccggacaacatgaaacaggtgaggtcaggtcactgcggacatcaagggcggacaccaccaaggggccctctggaacttgagaccactggaggcacacctgctatacctcatgcctttcccagcagccactgaactcccccatcccagggctcagcctcctgattcatgggtcccctagttaggcccagataaaaatccagttggctgagggttttggatgggaagggaagggtggctgtcctcaaatcctggtctttggagtcatggcactgtacggttttagtgtcagacagaccggggttcaaatcccagctctgctcttcactggttgtatgatcttggggaagacatcttccttctctgcctcggcttcctcatctgcagctacgcctgggtgtggtgagggttctaggggatctcagatgtgtgtagcacggagcctgctgtgtcctgggtgctctctacgtggtggccggtagaattctccatctatccaggctccaggagacccctgggcatctcccacctgtggcccctaaacccagagtgactgagagcacttaccattcagcttgtctcatccccagtctacctccttccttctaccctcactgcctcccagtcaggagagtgagctctcagaagccagagccccacccaaggggaccctggtctctccgccttcacctagcaatgggaaccctgcttcccaggggaggaaccaactgctccaccttctagggacccagtttgttggagtaggacagtaacatggcaggaatcggacttctgggcctgtaatcccagtttggatggcacgttagactcttggttgaccgttgtggtccttagaagtcccattctcccttccagttatgagaaaccaatgccttctagattcaggtgactatccttacctgggggtgctgatgcatcctcagttaacctacacccacctgaatatagatgagcgtagctgagttttcacccgtaggaccgaagtgttttgtggtggagtatctgaacaaccttggctctgtggccattcaacctgccaggactaacatttctggatttgtgaagaagggatcttcaaagccattgaacccacagagctgtgttgctttaaagccaccacaagggtacagcattaaatggcagaactggaaaagcttcttagggcatctcatccagggattctcaaaccatgtcccccagaggccttgggctgcagttgcagggggcgccatggggctataggagcctcccactttcaccagagcagcctcactgtgccctgattcacacactgtggctttccacgtgaggttttgtttagagggatccactactcaagaaaaagttagcaaaccactccttttgttgcaaaggagctgaggtcaagggtggcaaaggcacttgtccaaggtcgcccagcagtgctgctctgatgacttgtgcacatccccaagggtaagagcttcgatctctgcacagccgggccaacctctgaccccttgtccatgtcagtaaaatatgaaggtcacagccaggatttctaagggtcaggaggccttcaccgctgctggggcacacacacacacatgcatacacacatacgacacacacctgtgtctccccaggggttttccctgcagtgaggcttgtccagatgattgagcccaggagaggaagaacaaacaaactacggagctggggagggctgtggcttggggccagctcccagggaaattcccagacctgtaccgatgttctctctggcaccagccgagctgcttcgtggaggtaacttcaaaaaagtaaaagctatcatcagcatcatcttagacttgtatgaaataaccactccgtttctattcttaaaccttaccatttttgttttgttttgtttttttgagtcggagttttgttcttgttgcctaggctggagtgcagtggtgcgatctcggctcactgcaacctccacctcccgggttcaagtgattctcctgcctcagcctcccaagtagctgggattacaggcacccgccaccacacctggctaatttttttgtatttttagtagagatggggtttcaccatgttggccaggctggtctcgaactcctgacctcaggtgatccgcccgcctcggcctcccaaagtgctgggattacaggcgtgagccaccgcgcccagccaaaccttactatttttttaaagaattttttccagagtttaatttctgacatagcttaagttttccagtaactctaaactccatctcctttatcgtcattaagtcattcacaaaaagccaggagaagcatttggaaagggcatgataatcagtataata

SEQ ID NO: 4 - protein, 520 aa, Homo sapiens
MAGPERWGPLLLCLLQAAPGRPRLAPPQNATLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 5 - protein, 520 aa, Homo sapiens
MAGPERWGPLLLCLLQAAPGRPRLAPPQNVTLLSQNFSVYITWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 6 - protein, 520 aa, Homo sapiens
MAGPERWGPLLLCLLQAAPGRPRLAPPQNVTLLSQNFSVYLTWLPGLGTPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 7 - protein, 520 aa, Homo sapiens
MAGPERWGPLLLCLLQAAPGRPRLAPPQNVTLLSQNFSVYLTWLPGLGNPQDVTYFVAYQSSPTKRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 8 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTRELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 9 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKNDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 10 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGKVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 11 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWLESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 12 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEFLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 13 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPILVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 14 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANANYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 15 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPADLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 16 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNRTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 17 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPLTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 18 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPVASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 19 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSAKTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 20 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKWSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 21 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAK

SEQ ID NO: 22 - DNA, 34757 bp, Homo sapiens
aggaaggaaggaaggaaggaaggaaggaaggaaagaaagaaagaaagaaagaaagaaagaaagaaagaaagaaagaaagaaagaaagaaagaaagagaaaggaaggaaggaaggagaaaagaaagtcaacagtcaacatttcagagatcccaagataccaacactgaccgtgcctgctgctcttccatcctcctccaccctgcgcctttgaggtggaattgcgtcctctgtgagcagggctttgttaagagatcctaattaaggccaggcacagtggctcatgcctgtaatcccagcactttgggaggctgaggtcacctgaggtcaggagttcaagaccagcctgcccaacatggtgaaaccccatctctacaaaaattagctgagcatgatggcaggtgcctgtaatcccaactacttgggaggctgaagtgagaaaatagcttgaacccaggaggcggggttgcagtgagccaagatcacactattgcattccagcctgggcgacagagcttttgtctaaaaaaaaaaaaagaaaaaaaatcctgattaagcagaagccttgatgctagtcccagaagcatcctgaaatttccaaaagaaatttcccccgcggttaaactcagagcaacttttggacccaccaagctctgtgaaaatcattttctcttccaaaaactgatgggaccaaagctgatcccagtttcaaataattatcaaaaaattggaaacgaaatatgatcagaaaagaagaaagttgaaaaagaaaatcctcatcacccaaagacaacaaccattaatattttggtaattattattccaaatatctttctatgcatacagacagactgacacacacacacacacacacacacacacacacacacacactttttttttttttttgaaactgagtttcactctgtcgcccaggctggagtgcagtggcgcgatctcggctcactgcaacctccgcctcctgggttcaagcgattctcctgcctcagcctccctgatagctgggattacaggtgaatgccaccacgcccggctgattttctgtatttttagtagagacggggtttcaccatgttggccaggcttgtctccaactcctgacctcaggcgatccacccgcctcaccctcccaaagtgctgggattacaggcgtgagccaccgcgcccggctacacacacacttttttaatgggcctatgttttagcactcgcttttctgtttctcagtgtgttgcaaacacctcggtgtcgatacacaccattcggcaacgtcctcctaaagggccgcataatattgcgcgtcgtggcgtgtgccttactgggaagctactgctgtccaggtgaacaccacagccttcggggtcagaaagacagctttccccagaacaagcacctgaagctctggggcctgccgctccccgggagagaagtacgtggagaagggcagcacggatccgccgggatccccgggggcattaaagggaatcgcgtgtgtaaggcgcggagctcagcatccggctcagaaacgcgctcggatcccgccaatggcattgaggccgcgtagccaaaccggccttgaactctccctaatcctgccaaaatggcccgtcctggagcactggactggccgtgggttattgatcatcagccggtttcttcccctcccctgcccttcccccgtgcacggatttactgatttttttttccgggaattgagtaaaacaaaactaagtgcagatgaagcagaggtacgggcgagtttcgagcgcggggaccggcgcgctcccccccccccctccccccgcggcggggctgtccccagggaccttctcagtgaatcctaggcggcagggacgggcccgcggctctgcgggccattggctgccgactgcgtcacctgcccgcggtgggctaggagacgggaggcgggaggcgggaggcggggacctgggtccgggcggggacgccgcggcaggaaggccatggcggggcccgagcgctggggccccctgctcctgtgcctgctgcaggccgctccaggtaagggcgcggggccgcgggagggagggggaagagggctccccgggccgggccgcgcctaccctcggacccggagctcctgggacaggcacggggtccgcagccacccgagccgggtgcgaatcggccctgcctacgcgcccccagtttgcttcttcccaggactgaacagaaccgggtctttgatattcctctcccgcaggaaacgaatccagtttcctaatgcttccagcttcaggagaactggagaaaaaagacagcggcagtttgatactgcatattttttaataaagtgctttttaatgtttcctaaagaaagcactgatccctgcgtgaaaaccacacttgaccctaaagtgtggacagcagggaaagtgggaccgattgatgtcccttcccgttcctgccaggcctctggtgggacggagctctggtcgcctgtgccctgctttctaacaagacggctttcttttggtggtggttgttgttttgttgttgttttgttgttgttgttgttgttgttgttttcccacctctactgatgagtaaggtgtcaggtacaaaattcctcgccgtaggacccaaccaccaaacctcaccgcccacgactccaaccgaagcagggaagagaaggtccagaaatcgcccccaggatattttcctagtcttggactcacagtttaaagagctgtaaaggtccctgggcataatccaatcatcataaaagcctatatttattcagcaacttctttgtgccaggcaccgcattattctggaagcctcacgacccagccatcctaggaggtagatattatttttacttttccgatgggaaaactgaggctcagagcaattcagggaattcctcaagaaggacggcagaggtgaggcacacagaagagagaagaggggctaaagcaagcctggctagcttttgcctccagggtaggcacgtgggacaggctgtccatccactgggtcactaggccagccagggatgctccagcccccagtgcccacagcagcgttctctgtggctgatgagggaccgtgtacctgtgtgtggagggagggtggggtcttctgttcccctttcactgtcaagcccagaccttcttgtactttcacctgataagtatttaatatacacaacactaactatggtgtgatgatttaggagtaagtacagccagatctaagttcaaatactggctcccacacaaactgactgtgtagcctcaggcaagttagttagcatctgtctctgagcctagcgccctttccatggaagcagaatgaatgacacctaccccatagggtggtctgtcccaagggtgattgaggttttacatgtaaagagccaaactagtgcctggcatcctttgaaggcttcatagaggaaagttgctctagctgctgtttttctcatgtgacctagctcgaatctggggactgtcctgcccataggataccttacaagtggcttgcagacagcctggtctcctgctggtcacccgttaggaagtccagaagctgggagtagtaatagcactagcctcgtggtgatacagtcccagctagaggacacaggatgaggtggaagcaggcacccacttttgggtctaaaaggtgatgggtaggcagccgaggctggggacagccatccacagaactggaccctccctccctgatgccattttgcaacccgtatggatttccatcatggcacatgggacacttcaggaccctgaattctccatgggaccatgagctcctatagggcaggaatgaagttgtgttcttctttgaaacccctggcacaccgtggtcaacagatcttgtttgactcgtagtggtcaatagatggaatagttggaatcataaagctcaatagaccccatgagaacctagaagacaaagtacagtcaagagctcggactttggagttggctaggcctggactgaatctgattctacaacttaatagctgagagggccttggttttcccatctgtaaagattataattattataatgaatacctacctcctagggatgtaatgaggattaaaagagaaagtgcaggtaaactgtttaacacagaacctggctcatagaacacaatacacattagctgctattattattattattattttatttatttattttgagacagagtctcactctgtcacccaggctggagtgcagtggcgcaatctcggctcactgcaacctccacctatcgggttcaagcaattctcgtgtctcagcctcccaagtagctgagatgacaggcgtgtgccaccatgcccaactaatttttgtatttttagaagagacgtggtttcaccatgttggccaggctggtctcaaactcctgacctcaggtgatttgcctacctctgcctcccaaaatgctgggatcacaggggtgagttaccatgcccggccttagctgctattattatcatcatcgttatcatcatcatcatcacctcgtagatatgtcaaggaagattccctggaggaagtgacatttgaatcaagtatttcaaagactagatggtgaataccaggcagtcaaagacacctgggtttaaaaacatccagaagaatgcagtggcttggcaacatcgagcaggaagattgcctgatgagcctgtagggtagctgttggggagagagcagcaagacggcctggccaggccaggccaggccacgtcaggcagggcctcacaaacctcaataacaaatgtggactttattctgaggccaaggaaagggcatgaaactggggagtggtgtaatcagatgcgtatttcagaagatgaagattaacagtgagaaggaaaatgtgccacagaggggaatagaggtcagttaaagggagtcagggaaagtgtcctcgagacagtgacatcaaaggaatgtgaaaacagcaaaggagtgagccaggtggatatccaggggcagaactgttaaggcagagggaacagcatgagggaacagcgtgtgcaaaggcctggagttgggagtgtggctggggtgctccaggaagggcaaaaagtcctgtgtggatggagatatgggagcaagggaggagtggtgggtcagattgggtagggccttggtggtgattgtaaagactctggagtttagaccaggcacagtggctcaggcctgtaatcccagcactttgagaggccaaggtgggcggatcacctgaggtcaggagttcgagaccagcctagccaacatggtgaaacctcgtctcaactaaaaatacccaaattaaccaggtgtggtggcacaaacctgtaatcccagctactctggaggctgaggcaggagaatcgcttgaacccggaaggtggaggttgcagtgagctgagattgtgccactgtactccagcctgggtggcagcataagactctgcctcaaaataaaataaaaataataaagacttttgagtttccctggagtgagaggaaagccttagagggctttagcaggagatgaacatgatctgattttcatttttaatccttcctgctatgtggagaatggactgaaggcaaggtgttttgtatatttgtctgtttcgtagagacagggtcttgctctgttggccagactgaagtgcagtggcacaatcacggcagccttgaactcctgggctcaggcgaaactcccacctcagcctccttactctcaccattgtgccctgctaattttttaaaaaatttattttgtagagatgtggtctcactatgttgcctaggcaagtcttaaattcctggtctcaaatgattctcctgcctcgatgtcccaaagtgctgggattacaggtgtcagctgccatgcccgacctgtatttttttttttaatggggaaaaagccttttaatagtatgaggtgttttctggtgtttctaccataaagctcttctgtaaatcaaaatgagaatgtaattattgatagagcaatgaccttagactacagtgcagacttttcatcttacatttgggctcatgaattttagtataactgattatgacagtgttttttacatagttatgatctagagcagaactgaaaacaaaataacacatactctacatcaatatattcgttcagtaatatctgggcttggatgaacctgcagaagtaggtaaagctgtcagatattttcttaaaccaacagaaaagaaatgtatatgacagatgttgtgtttacttacttatttatttatttatttatttatttgagatggagtctcactgtgtcaccaggctggagtacagtggtgtgatctctgctcactgcaacctccacctcccggattcaagcgattctcctgcctcagcctcctgagtagctgggattacaggcgtgcaccaccacgcctggctaatttttgtgtttttagtagagacagggtttcaccatgttggtcaggctggtctcgaactcctgacctcgggatctgcccacatcagcctcccaaagtactgggattacaggcatgaaccaccacgcccagcctgtatttatttttttaccactatggagtccaatatgaaattctcacaactatgcatatacattattaacatgtaagcacacctaggtataaatatgcacatagtccattaattacatcaggggaattaaaaacatactttcaagttaaaatgaattttcaggaaaaaaactgcattcacaaatctgaaatgtgaatacaaaaatgaaattgtgaaataaataatgaatataggtgtcacctaaacttccatagtaacatgcctccaaatgtggatttagtgatcatccaccttgggacaagggcttttgagagcctccagctaaattagggttccagtagcagagtggctggcaagcctgccctaatgaataatgccagcgagctgggcgtgggtacttacagtgtgcccttcatggaatacttttttttttttttttggaatggagtctcgccctgttgcccaggctggaatgcagtggcacaatctcagctcactgcaacctcgtcctcctgggttcaagcaattctcgtgcctcagcctcccaggtagctgagactacagccctgtgccatcatgttctgctaatttttgcatttttagtagagacgaggtttcaccaagttggccaagactggtcttgaattcctgacctcaggtgatctgcccaccttgacctcccaaagtgctgggattacaggcttgagccactgcgcccggcccatgaaatacttcttacctggcggacagcctaatagcctagctgtctaacccatggctgggggtccttcacacttgtttatactggcagacgtccctgtgactcttgtctgatccatgtccaagtttatgcctgtctgaccattgctctggcgctgggagccagactgtgttcccagcaacccagggaaaaccaggcctgggctgggcctgggttcctgagatggaaggtgcaaattcagtacaccacctcaatgcaaaacaagttcaaaggcttattacttacagatcctgagcagggaaggtgcaatgagtagggagggtcatcctccatcctgggctacatgaagcgggaatgaagagtcaggcaaaaagaaagtgagagcttgtggcaatgagaagtatattatgtaagggactagggtgtgggtcaggttaagtttgagggcaaatgcttgaatgatccctttaaaggaatgggtgggaagtggggagcccagtttgccgggagggagagatgcctcgaagttcttatctctggccactggcttggaccatctgagtgtggcatctacttctaatgcctaggcagcaacctttgctgtgtcatctcccttacacaaggttggaagcaaggagaccggtcaggaagcctttggtgtaacccatgttattgtaatattcattcatttactcaacagatgtttattgtgcacctactatgtgctgaggccatggcaggcaggctctggggatgtggctgagaacaggacagagcccctggtccttgatatcctcaaggatgctccctcctggaggccattaggttcctgttccatggtgttctgctggaaccctccggtcccagagtgtgcaggagcctcccctcctggcaaagggtcttctctcatggcacaagggctgcagtacagccagtcagtggctcctggttcctcaaactcagtgagcacttgcctgcccttcgtgctgcccctcagcttgggatggcctgagtcaagaccagccaggagctccaggcttcatgacccctttctttcccccagggaggccccgtctggcccctccccagaatgtgacgctgctctcccagaacttcagcgtgtacctgacatggctcccagggcttggcaacccccaggatgtgacctattttgtggcctatcagaggtagaggggactctctcggctggtggatgggaagactgagggggtgggtgggggctggaggggcttctctgggacagctgcacccagtgtgggcagcactggctagctctctgggccctacgggagatggcatgtggccggcatttggagaggggcttttgataaaggtctggaggtggggaagatgttgaatgaagagcagtgtacaggtgaccagtctgccggggcgggggtaagtctttgaggaaagttggtgtggggcatggatgtagctgtgggggccagaggatgaaattctcaagtggctggatgaggtgcttggagctgtcccagctgatcagtgaggcaactaggtacacggcagaggagctgttacctgggcaattaggcatccctcaatgatcacactttttttctcttttttttttttttttgagacagagtcttggtctgtcacccaagctggagtgcagtggcttgatctcggctcactgcaacctccacctcctgggttcaagtgattctcctgcctcagcctccagagtagctgggattacaggcatatgccaccacatctggctaatttttgtatttttaatacagacgaggtttctccatgttgcccacgctggtctcgaactcctgagctcaggtgatccacccacctcagcctcccaaagtgttgggattacaggcgtaagccaccgcgcttggccaaatggtcacacttttcccgatgggatcattctcaatttggaagcccaggcagccacagcgaatccagagaaatctgacaatggaagcagatccaccatcttcgaacatagatgggaatcgttcagagttctttagcaggacagtgagatgatagaagcagaagctcgggaggattcacctggagttggtgaggaggggaaagcaggaagaggaggggaccaccgtgtcctcaggacccgtcctgtgccaggccaagtgctaagggccctacgtgaatatttcacttccttctcccaatgtgaccaggcaggctctgtgttttccccattctagaggtgaggggattgagctcagagggtgctgtgtcttgtctgaggaaggacgtcatggagccagaaggggaactcgggtccgactccaacatttgtgcccttcctgttgcatcacgtcatccttccatgtgtggaatccacatgtgagtgatgggagcctggcttgagcagggacagactgcaagagagctttcaaaagcaagagcgttatcaggtgccagaaaacacctaatatttactgtgtggctggcactgtgtcaacacatgtaatgaacttaatctcacagcagctctctgaggacaagttcagtacgcctctttacagaggaggagactgaagcaccaagggtgcatgttgctcaaagtcacacagctgggcgtagtatggctggaataaatttattaaggagttgaaagtctatcctctaggaccaagcatggtggcttacatctgtaatcccagcactttgggaggccgaggtgggtggggagattgcttgagtccaagagttcgagaccagcctgggtaacatggtgaaaccctgtctctacaaaaaaaaaaaatacaaaaaattagtgaagtgtagtagcatgtgcctgtgttcccagctacttgggaggctgaggtggggaggatcacttgagcccaggagatggaggttgcagtgagctgagatcacaccactgcactccaacctgaacaacagagcaagatcctaaaaagaaagaaaatctatcctctgaacttctatgatatttttcatgtcttttatacattagaatggtgatattctaattatataatttttttcatttgttagttggaattattttataaagagatgtatcctctcatctggtatttgatatccagtcatactattcaaataggcaagagaggataaatgcttaatttttttcctttatcaattttcaagataatgaattggttccttatcatctcccaaaggtgattgctagtttattattatcattatgaactcaggcatttaaacacatttggtggtttcagtctattgcgacgtactctgctcattgaagcttgaattgcctcatctctgtccagtgggagtctcatcaagtttgctcctgagtccttttaacttgaccctagtggtcaagttaaatctttccagatttaacagatacctttccagctgtccattacgacaagatgttccaggtccctctggtacaattcctgacctaaaacctgcagtcagccatttctccatttagtaagaaatggttataaagactataatctgcatgctagctatgctgatcactacttagctattgcttttggtgttttcagtgaacagagtgatgtgtgtataccacatagacacacacatgtacatactttttttttttagacagagcttcactctgtcacccaggccagagtgcagtggcatgatctcggctcactgcaacctccacctcctgggttcaagagattatcctgcctcagcctactaagtagttgggattacaggcgcccaccaccatacccggctaatttttgtatttttagtagagacggggtttcaccatgttggccaggctggtgtcgaactcctgacctcaagtgatctgcccccctcggcctcccaaaatgctgggattacaggcatgagccatcgcacccagcctacatgtacataatttttaagataaaatgcctaatgagttatacgggtgcttcccatctaaatttagttccttaggatttttacctgacttctatggtacatctatattttctttctttcacactgagaatcctgtttctcaaggacaggggacatgatagaactagaatgacccataattactcattttctttatcccaaaacatacatacttgcctcttaatagtttcttgctcttttcgcccaaagggtttgtgatggtcaatattaggtgtcaacttaattgggttgaaggatgcctagatggctgttaaagttttgtttctgggggtgtctgtgagggtgttgccagaggagactgacatttgagtcagtggactgggaatggaagactcgtcctcactcagtgtgggtgggcacaacccaactggctgccaggctggctggaaagcaggtggcagatggtgggatagcttcgcttgctgggtcttccagcttccttctttctcccgtgcgggatgcttccttctgctcctcctgcccttgaacatcacactccgggttttttggcctttagactcttggacttaagttagtggtttgctgggggctctcggatctttggtcacagactgaaggctgcactttcagcttccctggttttgagggtttcagattcggactgagtcactatggcttctttctttcccaccttgctgacggcctatcgtgggacttcgccttgtgatcgtgtgagccaattctccttaataaactccctttcatatatacgtataacctattagttctgttcctctggagaaccctgactaataaagggttgttgctttttctttaaaatctagtaattttatttgactgtgtgttggtattgctcattcattctgagttgatatttttaggcactcaatattctcacttaatacatggttccaaggcatttttattttaggaaggttttcttaaattatagttttagtatttgttctattctcttgttttgattttcttctttagggactcatatcacttgtatgttggatcttctttttctgtgttcagtatttgtcttttgggcacagagactcacacctataattccaagactttgtgaggcataggtaggaggatcgcttgagcccaggagtttgagaccagcctgggcaacatggtgaggccctgtctcaaattaaagaaaaaggagagaatacttgtctttttctttcaaatgccttttatctgtctgtctatctactattctgctctctaaatgaaataggtttcactcttgagtttttaaaaaactgtgtgcttccatgtgtgagattattcaacatcttatttgtaatctttctcttggttacatttatttttcctgaaactctagtctgcttttagctgacatgtttgtagctaagagcgcacatttcttatcatagcttgccgtgctgaattaattccaattttcttttaaaaccaacattattgagttaaaatgtatatagaataaactgttcccattttaaagtatacaatttgatgagttttgacaaaagtgggcacccacgtacccaccaccacaatcaagatgtaagacgttctctatcaccccagaaagttccctcatccactttgcattcaggcctccagatctaggcaaccacagatctgctttctgacactgtggattaaactttgcctgttccagaatttcatataaatggatgtgtatagtatgtaccctttcgtgtctggctcctttccctcagcataatgtttctgaaattcacccacattgttacatgtatcagtagttaattcctttttattgctgagtagtaatgccattgtatgactatgtatgacatttgttaatccattttcccgtcagtggatatttgggttgcttccagttctgggcaggtattcatttgctagggctgccatatgcttgccctctggcctcccaaaatttgtgtccttttcatatgcaaaatacattcaccccctcccaacagccccaaaactctcttttttttttttttttgaaacagagttttgctcttgttgcccaagctggagtgcaatggtgtgatctcggctcactgcaacctctgcctcccgggttcaagagattctcctgcctcagcctcctgagtagctgggattacaggcatgcgccaccacgcctggctaattttttatatttttagtagaaatggggtttcaccgtgttagccaggctggtcttgaactcctgacctcaggtgatccgcctgccttggcctcccaaagggctgggattacaggcatgagctactgcacctggctagccccaaaactcttaacccatttcagcatctactctaagtccaaagtctcatctaaatcaggtatgggtgtgactggaggtgttactcatcctgaggccaaattcctctccacttatgaacctgtgaaaccagacaggttatgtgctttgaaaataaagtgatgggacatgcatgggatagactttcccattccaaaagagaaaaataggaaagaaggaaagagtgacaggtcccaagcaagtctaaaacctcgcagggcaaattccattagattttaagtttcaagaatagccctctttggctcagtgctctgccctttgggcccactggggcggcagccctatcccctttgccctgggtggtgaccctaccctcgagtcactggttagcagcagcctagcctgctgaaactaaggaggggacagtgttgcctccaggtctttggtggcagtgacaaccctgctgatctctgaatcatcttccaggaaatttttccctatacttgaaggatattgcgtgttcacagccaaatagctccagctcttgtccctttctttagaatcccagaagtccaacagccttccttcattctgtcccatctctgtcccctttagtcaaagctggaagtgcctctgctggtataatcccatcagtatgtctaatttctgcttaaatggctgattaagtctatgagttgcacctctgatctctttatcaaaaggttgttctagccacaaccttagtgtcctccccagaacatgctttctcatttttttttttgcaatgtggataggctgaaaattttccaaagcttcaagttctagttccttttggcttaccaattcttttcatatatctcttctctcacattttactataagcagtaagaagaaaccaggttgtaccttcagcactttgcttagaaatctcttctgctaagcatccaagtttatgtcttttaaattatctttttgttatttattttatattatcatttttgagatggctagccaatgatcttttaacttctaatttctgcaaaacactagaagacaattcaaccagttctttgccactttataacaaggatcacctttcctccagtttccaataacacattcctcttttccacctgagacctcaccagaatcacctttaatgtctatattcctaccaatagtctttttaaggcaatataggctttctctaacatgcacttcaaacttcaagattctacccattatgcaattccaaagccacttccacatttttaggtattgattacctcagcacctcatttctggtgcccaaatctgcactggtttgctagggctgccataacaaagtacgacagtctgggtaaacaacagaattttattttctcaaaattctggaggttggaagtccaaggtcaaggcgttgctaggtttagtttctcctgaagcctctctccttggctagcagatggctgccttcttgctgtgtcctcacgtggctttttctctgtgtgtgttcactctggtatctcttcctcttcttacaagtacaccagtcctactggattagggccccagccttattacttcatttaaccataattacctctttaaagctcttatctcaaaacacaataccactggggatgaggtcttcaacatatgaattttgggggaactcaattcgtccataatagggctattatgaattaagctgctgtgaacattcatgtacaagtctttgtgtggatatgttttcatttctcttagataaagatctaggagtatcagcctgggcaacatagtgagaccccatctttacaaaaaattttcaaaattagccaggcatggtggcgtacacctgtagccctgccatctcaggaggctgaggtgggaggatcccttgagcccaggggttttagactgcagtgaactatgattgcaccactgcaccccagcctgggtgacagagtgagactctgtctctaaaaaaaagagagagaggggaggaaggaaagaagaaagagagggagggaaggagggagggagggagggagaagaaaaatggatctagggttaagatttaggagattaggtaatgaatgtgtactattacagggaactgtcgagctgtttccaaagtgactgtaccattgttcattgccaccaacaatacatgagagttctagttactccatgtgcttgttacacttagtattatcagtctttttcattttaaccattctagtgagtatgtagtagtattttattatggctttaatttacaactccctaatgatgaatgatgttgaacatcttttcatgtgcttattggccattcatatatcttttgtgaagtgactattcaaatatttttccactttttattaggtcatttattttcttattattgagttatctatgaatacaaatcctttatcagtgtatgtattgtgatttttttccccagtggctggccttttcattttcgttaggcttttttggtgggtttttttttttttttttggaagagaaaaatattttaatttgataaaatccagtatatcaggtgttatagactgaattatactctaccccacaaattcatatgttgaagccctaacctctaagtgactatttggagatgagcctttaaggaggtaattaaagtaaaatgagatcataagggtgggccctaatctaataggactggtgtctttataagaagaggaagacaccaagagcgcatgcacacagaagaacggccttgtgaggacacagcaagatgacggccatctgcaagccaaggagagaggcctcagtagaaaccaaacctgctgatgccttgatcttggacttccagcctccagatttctgttgctgaagccaccctgcctgtggtgtcttaccatggcagccctcacagactaatatatcagatttttttccttcaacagttaacgcttttggtgtcctaagcaatattcgcctgacccagggtcatgaagatttttcttctatgctttcttctggaagttctataattttagcttttacatatttttttaactttccttcttcttgccttctgtttcttttaaggcatcatctattgtgttaatttgttcttgtattccttctgatttattcttcacttctgaaatgaattttgctttttaaaaatatatataattcttttctgtgtctgagtttttctaattaggttttatgtggttttttcttgtcctgcatcactttttactgtcttttgcccattttgaagtatcaggttccagttttgatctgttcatggatatgtttttgtgacatgtttcttctggcttcttatcatttattgcttagcttattaatttctattctttcttattttctattataagtatttaaagctatatgttttcctctaagtattacttagctgtcttatacgttttcatttgtgttatttggtgatcattcactttcagctatttattaatttccattataattctttcatctatgggttgttttaaaaaatatttttaaggccaggtgtggtgactcacatctgtaatcacagcacttagggaggctgaggtgggaggattgcttgaggccagaagtttgagaccggcctaggcaacaaagtgagaccccctctctacagaatatttttttaaaattagctgggccaggcgtggtggctcatcccagcacctgtaataccagcactttgggaggccaaggcagatggatcacctgaggtcaggagttcgagaccaccctgggcaacatggtaaaaccccatctctactaaaatataaaaattagccaggtgtggtgataggtgcctgtaatcccagctacttgggaggctgaggcaggagaattctttgaacccaggaggaggagtttgcagtgagccgagattgcaccactgcactccagcctggatgacagagcgagactctgtctcaaaaaaaaaaagaaaagaaaattagctgggtgtagtggcaggtacctgtggtcccagtgactcagagactgaggcaggaggatcacctgagcccaggagtagaggctgcagtgagctatgtttgtgccactgcactccagcctgtgcaacagagcaagacgctgtctcaaaaaatatatatttttttaaattttcaaacttcctttagttctctttttgttattaacttttaactgaatgttttgcaatcagaagaaatactttatgagatacctattctttaaaatttcttaagaattgctttgtgttaatattttgttaatagttcacatgtggttcaaccaatttgtttagttagttctgtatatgttcattagaccaacttgataactgtgttgttctttatttatttatgtatttatttttctttgtctattcatcaattgctgggtgagatgtattaaaatttcttgttgtaagtgtggctgttcactttctacctgtagtttgtctgtttgctttatagagggtgaagttgtttagtaggcacacataagttagaatttttctgtcttcctggtgaatggaatcatttatcattatctaatgttcttttcatctttagtattgctttggacttggaagtctgtattttgtctcctgttaatataactacactggttcctttggtgtgaatatttgcatagtataacattttccatgaagaaacaaaacagaggaattggttctttctcaaaatctgatctttgtgtcagcccccatctcagccttctccattcatccttggtcactccccaaacccaggagcaatccttgattctccttttccccacattctacatccaatccgttagcaagttctattagttctattattacctccaaaatagatattgaatccagccctttctcactgtctccaccatcatcctgtctcacatccctaccatggcctccttgctggttgaccagagtgatcttgtaaaaacatgttaggccaggcacggtggctcctgcctgtaatcccaacactttgggaggccaagcgggtgggtcacctgaggtcaggagttggagaccagcctggccgacatggtgaaaccctgtctctactaaaaatacaaaattagccaggtgtggttacgctggcctgtaatcccatctactcgggaggctgaggcaggagaatcacttgaacccaggaggcggaggttgcagtgagccaagatcatgccactgcaccccagcctgggcaacagaacaagactccatctcaaaaaataaaaattaaaataaaatgttaggctccctgggtctctggcttagtccatttgtactgctttaacaaaataccttagaatggtgtaattctaataattgctattaataaataatagcaattaataaataatagcaatttccttctcacagttctagaggctgggaagttcagggtcaaggtggcacctgactccgttctggtaagggcggctctctgcttccaagatggtgccttctcgctgcgtcttcgcatagcggaagggcaaacactgtgtcctcacgtggcagaagagatagaagggccaggcagctctctgaagtatccaggttggagtcatggacctgcatgttcccctctgacatccacagagtacctatcatggtccttggcatgcagcaggtggcccataaacgcctgaatgaacaaacatatagtaatggtcgctagtactaggaatagcagccaccgcaacagtcctgtgagggaggcattacagatgaggaaactgaggtttaggggcaaggacctgcccatggtcccaaagctagggagggacagggctgggattcccactcccatccatctggctccagaacctgagctcctgaccaggctgttcttatcctgtctcagccagtggctgcctgtctggacggatggacctaaagtcagtccagccaaacagagggaagcatgatcaactgttctctaagttccctgacccggagaggctgagtccatggcccaagctctcctctctcctcccccagctctcccacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaaggtaggtctgtgggtaagggactgagtggaaggctgtccatcccatcggggagctgtgctcagtgctcagtggttctgttctcctgaccatctgtctcccacttccccaaagcagagggcagctccctgggccaggccctttgagatggggtgtgggaccagcaacagcgagggaccatgtctggcagcctgtcagggagttaggggagctccagccagcaccagcaatctcacgtgcaccctctgctaacaatgttcattattttcagttgagcaccattttggtcatggactacacaaggcactttatatgcttattcctatttttttatgttcagcttctctccttaaaaacaatgtttaaaaccaattctgggccaggcgtggtggctcacgcctgtaatcccagcactttgggaggccaaggcaggtggatcacctgaggtcaggagtttgagaccaccctggccaacatggcaaaaccccgtctttactaaaaatacaaaaattagccaggcttggtggcaggcacctgtaatcccagctactcgggaggctgaggcaggagaatcgcttgaacccaggaggcggaggttgcagtgagccaagatcacgcccctgcactccagcctgggcgacagagcgtctcaaaagaaaaaaattaataaacaaagaaaaaaaaacaaattctgtttgcaaaagtattttctatacactgtagaaatttgtggggtgtgggggggtaaagatgatagaaaaaaaaatgtcccatgcttactggcagaaatcatgtattgacattgggtgaggagggcactttttttttttcagtctatttttaatcttcacagcaaacttgtgaggttcatttccatcaacctgagactcacagaagctaagaaacttgataccgctagtaaccagtggacttgataccgctagtaaccggtggacatagatgtgaactggatctttctgacctcgggcagggccgggtaacaaggggaggataaatgcccagacagtgtcctcagagagctgagagctgtaacttgctgcccgggcttctcacagtgttcaaggacaaaataaggctttaagagagaagagggacagactgattgcagggcagcaggaagagatggtagagaaggaagaagagatgattcgtgtggaaagaagctggctcggtggatggataaaagaagggaaggacagatgggtaagaagaaagggaggatggaggggatggaggaggaagcaatggaaaaatgggaaggaaggaggttggatggaaggatagatgcctattaggaaggaaatatgtgtggatagagagatggaggataggaagtatgttagtcaaggttctccagagaaactgaaccaataggatatatacagatacactaagaggaggccagccgggcgcggtggctcaagcttgtaatcccagcactttaggaggccgaggcgggcggatcacgaggtcaggagatcaagaccatcctggctaacacagtgaaaccccgactctactaaaaatacaaaaaaaaattagttgggcgtgatgatgtgcgcctgtagtcccagctgctggggaggctaaggcaggaggatggcgtgaacccaggaggcagagcttgcagtgagctgagatcgtgccactgcacttcagcctgggtgacagagcaagactccgtctcaaaataaataaataaataaataaaaagaggccagccatggtggctcacacctgtaatctgagcactttgggaggccgaggcggatggatcatttgagatcaggagttcaagaccagcctggccaacatggtgaaaccctgtctctactaaaaatacaaaagttacccgtgtgtggtggcacacacctgtagtcccagctactcaggaggctgaggcaggagaattgcttgaacttgggaagcagaggttgcagtgagctgagatcacgacactgcactccagcctgggtgacagagcaagactttgtctcaaaaaaaaaaaatttataataagaggagatttattatgggaattggctcatgcaatcacagacacaaaaatgtcccccagcatgcagtcatgggctggacaaccaggaaagcttgtggtgtgattctgtctgagtctgaaggcccaaggccaggggagcagtggtgtaacccccagtccgaggccacaggcccgacaatcagaggggccactgatataagtcccagagtccaaatgccggagaacaggaagctccaacgtccaaggacaggagaagttgatgtgccagctcaggaagagagaatgtgaatgtgccattcctcctccattttttgttctctttgggccgtcagtggattggatgatgcctgcccacactggtgaggacagatcatcaccaaatctgccgattaaaatgttaatctcttctggaaaaatcctcacagatgggcccagaaataatgttttactgtctacctgggtatcccttagtgcagctaaattgacacataaacttaaccatcacaggccaggcactgtggctcacacctgtaatcccatcactttgggaggccaaggtgggaagatcctttgaggatgaggtaggcagatcacttgagcctaggagttcaagaccagcctaggcaacatagggagacctcgtctctacaaaaaaaaaaaaaatttaaattcgctgggtacggtggtgggcacctgtggtcccagctatctgggaggccaaggtaggaggatgacttgagcccaggaggtcaaggctgcagtgagccatgattgttccattgaattccagcctcggtgacagagcaacaccctgtcttaaagaaagaaaaaatttaaccatcacagaaggcagaagaaaaggcagatgggtggatgagatgggtgggtagatagtatagaagaaaagcgggacatccaggcagggaaggaagggctggagcgaaggagaagcaaggaaggaaggaaggagagacaagaaggaaggatgtgtagaaaggtggaagagaaaagaagaatggatgtatgggaagaatggatgagtaggttagaaggctcactggctagataaaaggtgagaagtataaatgaataataagaaaggaggcataggaagaaaaaaatattggttagaaaggatgattgagaagaaagggtggttgggaaggaaggaaggaaggatggatggatggatggatggatgggaaggaaaggaaggataagaaggcagacaggaaggctctctggctagaagaatggcagacaaaccacaataattgctgaatgggtaggaataagacattagaagaataaagggaaagacacaaagatatttaaaatgttttcattaattttttgcctcctccctgaatttctcctgattcttcagccccacatcccaagccagggtgatccttcctgcctttacactccctccacactttttctgctctcatatgtggccgtggtcactttcttttggtagtttgcatatttcatttaccccaaactttcagctcctgaaggtcaggatacaaggaggcctcatctccgcattcccctcagctcccttcctgaagcttgatacctagtcagtacccagtggatgtttcctaaacatgtaagtaatgacatcatgaagaagccacatgtttaccttgaccacaaacacagggcaaaggtgactagtgtggtcagagatccctgctggctgggaatcagggaaggctgcatggaagaagtggcattttagttagaacttgaaaggtggtgtatttagttttctctggctgccatattccttgtcacattgccctctccatcttcaagccactgggcaaggctagaaggccctcaacagactatcggtaggaatgtggaagttgaagactcagagtgcagaaagaaacaagtagcattttagagaaaagctaaatcccctccaagaatacctcaatcatcgtgaagagcctgttagtagacgcactaacactcaaggcactgcttcacaaggtaaggaacgtgtaattgaaaacttgagaaaggaagaaacttgttctgtactggcagaaagcttagcagaattgtgtcctgcagtcatatgggacacagagcttgtaaatgatgaatttgaatgcttatccgagaaggtttccaaataaaatgtggaaggcacggcctggtttcttcctgcctcttatagtaaaatgcaagaggagagagagaaaatgagggaagaacttaaacagaaaggaaccaggacttgatgatttgggaggttctcaacctatgcaaaaaacaataaaattaagagattgtagctgggcacagtggctcatgcctgtaatcccagcactttgagagtccgaggcgagcagatcacctgaggtcaggagtttgagaccagcctggccaatgtggggaaactccgtctctactaaaaatacaaaaattagctgggtgtggtggcgggcacctgtaatcccagctactcaggaggctgaggtgggaggatcacttgaacccaagaggcggaggttgcagtgagccaagatcatgccactgcactccagcctgggtgggtgacagagcaagactccatctcaaaaaaaaaaaaaaaaagagattgctcccaaaagtgtgacatagagaaacagccaagtatgtgattataccaaacttcaggaagataaaagatcaaagtactcagtcgctcaaaaggctctttgaagagattaagattataactcacagtccccttcaatcaaaccaggggacttctaggaagctgaacagcattgtccctcagccatatcagctggagccaaaagtagagaagggcttatctgaaaaaaggatctgtggacctggcttttatctaataatgcagtggattcccccatgacatccataggagacccgtaaagttcctgagacgtttacatccacagaaacactgttagcttggattaaatggaacacagagagtatgaaatcaaagaaggctgttggactctccagtttctactgttgagatgcagactggtaaaactacttagctgcaaacacctgctacctttagtgaaaaggaaggatatctcagacggtgaaaccagaagctcaaagggcagtgctaagagcgaaagagaattcttcccaggccttgaaacctaatggagttttcttggctggattttcaaactgcattggaccatgacctgattgtccctttcatgtccccatgcttgagccagattgtctgcaactgttatcctgtgcctgtcccacattttatgttgggagcagaaaactttagttttgctggcccacagatagagagaaactgtaccccgagagttgtactgactggactatgcccagagtctatttgactctgacttagatactgttgatttgggaatttgagttgatgctgtaatgagatgagactttgggggacattgggatggagtgaatggattttgcatttgaaagagatgtgggttgggtaatcctagcccacacctgtaatcccagcactttgggaggccgaggcaggcagatcacctgaggtcggcagttcgagaccagcctgaccaccatggagaaaccccatctctactaaaaatacaaaattagccaagcatggtagcacatgcctataatcccagctactcgggaggctgaggcagtagaatcgcttgaacccgggaggcagaggttgcggtgagccgagatcacgccattgcactccagcctgggcaacaagagtgaaactccatctaaaaaaaaaaaaaaagaaagaaagagatgtggattttgggtgggggacagagggaagaccatggtaggcagaatgatcctctaaaggtgctctgccctaatccccagaagctaagaatatgttagatgtcagtattgcgtggcagtaggaatcttaattaacgttatagactgttatggtttgaatgtcccctctaaaactcctgttgacatttaatcatcattgtgattgcattaagaagtggccctgttaaaaggtgatttagtccttaagaacgctgcccccgtgaatagattaaggtcagtcttgcgggagtgtgtttatcaagaatggattgttaaaaagtgagttctggccaggggcagtggcttatgccactcagcactttgcggggccaagacttgaagtcagttgtttgagaccagcctggccaacatggtgaaagtctgtctctactaaaaaatacaaaaagtgtccgggagtggtggcgggcgcctgtaatcccagctgctcaggaggccgaagcaggaggatcgcatgaatccgggaggcagaggttgcagtgagctgagatcgccccgttgcactccagcctgggtgatagagcaagactctgtctcaaaaaaaaaaaaaaaagaggaaagaaagaagaaagaaagagaaagaaagaaaagaaagaaaaggaaggaaggaaggaaggaaggaaggaaggaaggaaggaaagaaagaaagaaagaaagaaagaaagaaagaaagaaagaaagaaagaaagaaagaaagaaaagaaagaagaaaaaaagaaagaaaaaagaaagaaagaaaaagaaagaaagaaagaaagaaagaaagaaagaaagaaagaaaagaaaagaaaagtgagttctgccctctcttgctggcttactctcaccctctcttgcccttccacctgccaccatgggatgacacagcacaaaggccctcaccagatgccagtgccatgctcttggacttccaagtctccagaaacatgagccaaatacacttctgttcattataaattacccagcctgtgatattctgtaataacaacacaaaatagactgagacatagatcttcaaatagtgaggttatcctggataatccagatgggcccaatctaatcccatgagcctttaaaactttctccagatggaggcagaagagaagtggcagaaggggaagtcagagagatttgaagcataaacaggactccatggtgccgtttctggtttgacgatggagtggtaacgtgatgaaaaatgtgggtgccttccggagctgagaggctcccactaacaatcggccaggaaacagggaccacagccctacagccacaaagaactaagttttgctgacaacccaagggggcttggaagtgtcttctcccccatcggttccagatgtgagacccagagcgaaggaaccagctgagcccacctggacttctgacctagagaactgtgagataataagtttgtatcatttttaaggcactgtgtgtgtggtaatttgttatgacagcaatagaaaatgaatccagatgggcaggatctgccaggccagtgacatgtggagggcacccaggcggatgggatggcatgagagaaggcaggtcagcaatgagcttgcccaggtcacctctcctctctaagcctcagttttcctctctatgaaatgagagtagtgatatctccctcccagggtcagtgcaaggctgaaataacagattataaggtgctaggtgcacaagaagtgtttgaaacatgctagttgcttttccatttccaagagagctctctggtcttgggggatggaggcagtgcggcccctcgggattactgacaggtcctgctctgtttctgcagtggagccggccccacctgtcctggtgctcacccagacggaggagatcctgagtgccaatgccacgtaccagctgcccccctgcatgcccccactggatctgaagtatgaggtggcattctggaaggagggggccggaaacaaggtgggaagctcctttcctgcccccaggctaggcccgctcctccaccccttcttactcaggttcttctcaccctcccagcctgctcctgcacccctcctccaggaagtcttccctgtacactcctgacttctggcagtcagccctaataaaatctgatcaaagtatgatgacctacaggaggcctgcttgccaagtcaacagattcagtacagaaaaactgaaaaatacagataagctctaagaagcagaccaaaagtacccagagatgaccgcacatcactctggtgtatatccaatttcagatttgttttctgtgtatgcatgtgtgtatagctgcatttatttatggcaagggctggcagactttcccgaagaaggccagatagtcgatatgtttggcttcatgggccgtatgttcgctcaggactactcaacgctgcagttatagcacaaaaggagccgtagcctatacgtaaatgaatgggcatcgctgggttccagtaaaactgtttacaggccaggtgcggtggctcatgcctgtaatctcagtactttgggaggccgaggtggtgggaggattaccttagcccaggagttcaagaccagcctggggaacatggtgaaacattatccctacaaaaaaaaaaaaagctgggtgtggtgatgcatgcttgtggtcccagctgcttgggatgctgaggcaggaggatcgctcgagcccaggaagcaaggccacagtgagccatgatcgcaccactgcactttagtctgggcaacagagtgagaccttgtctcaaaaaaaacaaaaaataaaactttttacataaacaagtggccaaccagacttggtccctgggcctctgctcttgaatgttcttgcttccactaaagtaacattcacactcccgatttttgcatactctgggttctggggaatatagatccgaatccagcgtggttcctgccttcaagaacctcacaaatattctagaccagcactgcccaatagaaagaaatataatgcaagccacatgtgcagttttaagtgttccatgttaaattaagtaaaaagagacgggtaaatcgaattttaataacagattttacttcatccaattgaatggtatcatttcaatgagcaattctgatagtgattgagatcttttacattctttttcactacgtctttaaaatctgatgtgtgttttgtacttggaacacttctcagtgtggaccagatgcatttcacatactcagtagtcacgcgtggccagtgccttccataccacacagtgcagcatctgtagaggtttcctccactgctgatagactaggagaccccaagatggaaagcctgaagaatctgctcctcgaagtagggaccttaatggggtgcacgccagggcgaccccaagtggtaggctgcttttgaaccatggctatccctacctctagactcagctgaaaagaactcaggtagtcttgggaagtgcttcctcaatgcttaaactttaatgcaggaaaagaatagaaagttcaggcaaggagggaggatcacttgaggctgggagttcgagaccagcctgggcaacagcaagaccttgcctatacaaaaaataattttaaaaaattacccaggtatggtggtgtggatctgtagtccctagttacttggagagctgaggtaggaggatcgcttgagcccaggagtttgaggctgcagtgagctgtgatcacaccactgcactttggcctgggtgacagaaccaaaccctatcccctacaaaaaaacaaaaaaaaaaaacaaaaaaaaacaccctaccatgtctgccaaccccactctgtcctggctgtgtgaaaccagtccccacagcagctctgccactctctgcttcttttccaaacagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccaggtgggtatcaagtggtgcagaaggagaaactttccctctgggccttgggagcttcgtgacacagtggttaagaacatgagcctagagatagactcgcctggattaaaaccacactcattgtgtgtctttgggcagcttacataatgccccgaaccttggtttgcacagtctgcaggatgggtttattcttgtgaggattaaatagggtcatgtatgtgaagcactcggcacaggtgcagttgtagacaagagccattgttgtttctctcattgttatttttccttccttagaagccaactgggctttcctggtgctgccatcgcttctgatactgctgttagtaattgccgcagggggtgtgatctggaagaccctcatggggaacccctggtttcagcgggcaaagatgccacgggccctggtatagcaaatctgggggtgtgcggcaggtggggaggggttgagagtaagggagtggggctggagctatgagttgttcagatagaatatcaagatggtccagactcttggaccaaaacatctatctttgtgtctgaatttccaccattagtaatgcattcatttagtcctgaataaaatggcaaacaggccctggagggagcagtgccttaagttcctttgagataaataacttcacctctgctaaggatgtgtcagctgctgagagcagagcccctggccttggacctcaggagagacactcaaaaggggaggagaggaggcaccaaaggggacatcttaaaagagttccaatttttagttcacactttaacccaggataagctgtgtcctggctgaccttggagtttcttccctggtctgctgggtctctcccttagaacctaggggcgagctggggcaggggaagcccaggaggtgatataggtcggccctgttcagatgagggctggcaggggcagcttgggcatatgcgaggctccgatgggcatgggggctttgaggatggattctgagtgtccctgcatcgtggcagggtggcaaagggagcatttccaaatttcctggctccaggatctgtgggagaatcccactaactgtcagggtgacaacctcgggtagacatgtctgtgccctgccccgtgccctcagccttcctgttaagagcacaccagctggatttgcaactcccagcgcctgcacccaatgggctttctctggcctctggagcccacattgcccctgcatgtggcaggctgcaagtgtcacagccaccagctcttccattcctcaacaatgactgtgggtaaatagcccaggagcgtccccctcctgggatggttctgaggtgcgtgtgcccagtggctccctgagttgccagcaggattaagtgccagtagccctagtggtcagctgcttgataacaccctgcttcctggctgctcccccagtcccatctggtgtgttctgggatcatctcccaaagaaactgcttacacttgaagccttgtctgaggtctgtttctaggggaattcagatgacgataattatgcttcaggaaagcctaaattttctgcttttctctcccctacccaaatcaggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtgagctgtcccccgacatcccaccgaatctgatgctgctgctgcctttgcaaggactactgggcttcccaagaaactcaagagcctccgtacctcccctgggcggcggaggggcattgcacttccgggaagcccacctagcggctgtttgcctgtcgggctgagcaataagatgcccctccctcctgtgacccgccctctttaggctgagctataagaggggtggacacagggtgggctgaggtcagaggttggtggggtgtcatcacccccattgtccctagggtgacaggccagggggaaaaattatccccggacaacatgaaacaggtgaggtcaggtcactgcggacatcaagggcggacaccaccaaggggccctctggaacttgagaccactggaggcacacctgctatacctcatgcctttcccagcagccactgaactcccccatcccagggctcagcctcctgattcatgggtcccctagttaggcccagataaaaatccagttggctgagggttttggatgggaagggaagggtggctgtcctcaaatcctggtctttggagtcatggcactgtacggttttagtgtcagacagaccggggttcaaatcccagctctgctcttcactggttgtatgatcttggggaagacatcttccttctctgcctcggcttcctcatctgcagctacgcctgggtgtggtgagggttctaggggatctcagatgtgtgtagcacggagcctgctgtgtcctgggtgctctctacgtggtggccggtagaattctccatctatccaggctccaggagacccctgggcatctcccacctgtggcccctaaacccagagtgactgagagcacttaccattcagcttgtctcatccccagtctacctccttccttctaccctcactgcctcccagtcaggagagtgagctctcagaagccagagccccacccaaggggaccctggtctctccgccttcacctagcaatgggaaccctgcttcccaggggaggaaccaactgctccaccttctagggacccagtttgttggagtaggacagtaacatggcaggaatcggacttctgggcctgtaatcccagtttggatggcacgttagactcttggttgaccgttgtggtccttagaagtcccattctcccttccagttatgagaaaccaatgccttctagattcaggtgactatccttacctgggggtgctgatgcatcctcagttaacctacacccacctgaatatagatgagcgtagctgagttttcacccgtaggaccgaagtgttttgtggtggagtatctgaacaaccttggctctgtggccattcaacctgccaggactaacatttctggatttgtgaagaagggatcttcaaagccattgaacccacagagctgtgttgctttaaagccaccacaagggtacagcattaaatggcagaactggaaaagcttcttagggcatctcatccagggattctcaaaccatgtcccccagaggccttgggctgcagttgcagggggcgccatggggctataggagcctcccactttcaccagagcagcctcactgtgccctgattcacacactgtggctttccacgtgaggttttgtttagagggatccactactcaagaaaaagttagcaaaccactccttttgttgcaaaggagctgaggtcaagggtggcaaaggcacttgtccaaggtcgcccagcagtgctgctctgatgacttgtgcacatccccaagggtaagagcttcgatctctgcacagccgggccaacctctgaccccttgtccatgtcagtaaaatatgaaggtcacagccaggatttctaagggtcaggaggccttcaccgctgctggggcacacacacacacatgcatacacacatacgacacacacctgtgtctccccaggggttttccctgcagtgaggcttgtccagatgattgagcccaggagaggaagaacaaacaaactacggagctggggagggctgtggcttggggccagctcccagggaaattcccagacctgtaccgatgttctctctggcaccagccgagctgcttcgtggaggtaacttcaaaaaagtaaaagctatcatcagcatcatcttagacttgtatgaaataaccactccgtttctattcttaaaccttaccatttttgttttgttttgtttttttgagtcggagttttgttcttgttgcctaggctggagtgcagtggtgcgatctcggctcactgcaacctccacctcccgggttcaagtgattctcctgcctcagcctcccaagtagctgggattacaggcacccgccaccacacctggctaatttttttgtatttttagtagagatggggtttcaccatgttggccaggctggtctcgaactcctgacctcaggtgatccgcccgcctcggcctcccaaagtgctgggattacaggcgtgagccaccgcgcccagccaaaccttactatttttttaaagaattttttccagagtttaatttctgacatagcttaagttttccagtaactctaaactccatctcctttatcgtcattaagtcattcacaaaaagccaggagaagcatttggaaagggcatgataatcagtataataatt

SEQ ID NO: 23 - DNA, 27 bp, Artificial
  Ax5-1 oligonucleotide PCR primer
gctgcaggccgctccagggaggccccg

SEQ ID NO: 24 - DNA, 29 bp, Artificial
  Ax3-1 oligonucleotide PCR primer
ccaggtattcggactccacccagggggac

SEQ ID NO: 25 - protein, 231 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 26 - DNA, 27 bp, Artificial
  Ax3-2 oligonucleotide PCR primer
ttggttcccgcacactcttccacttcg

SEQ ID NO: 27 - protein, 130 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWERAKGL

SEQ ID NO: 28 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 29 - protein, 560 aa, Bos taurus
MLALLGATTLMLVAGRWVLPAASGEANLKPENVEIHIIDDNFFLKWNSSSESVKNVTFSADYQILGTDNWKKLSGCQHITSTKCNFSSVELENVFEKIELRIRAEEGNNTSTWYEVEPFVPFLEAQIGPPDVHLEAEDKAIILSISPPGTKDSIMWAMDRSSFRYSVVIWKNSSSLEERTETVYPEDKIYKLSPEITYCLKVKAELRLQSRVGCYSPVYCINTTERHKVPSPENIQINADNQIYVLKWDYPYENATFQAQWLRAFFKKIPGNHSDKWKQIPNCENVTSTHCVFPREVSSRGIYYVRVRASNGNGTSFWSEEKEFNTEMKTIIFPPVISVKSVTDDSLHVSVGASEESENMSVNQLYPLIYEVIFWENTSNAERKVLEKRTNFIFPDLKPLTVYCVKARALIENDRRNKGSSFSDTVCEKTKPGNTSKTWLIVGTCTALFSIPVVIYVVSVFLRCVKYVFFPSSKPPSSVDEYFSDQPLRNLLLSTSEEQTERCFIIENASIITEIEETDEIDEVHKKYSSQTSQDSGNYSNEDENSGSKISEEFPQQDSV

SEQ ID NO: 30 - protein, 560 aa, Bos taurus
MLALLGATTLMLVAGRWVLPAASGEANLKPENVEIHIIDDNFFLKWNSSSESVKNVTFSADYQILGTDNWKKLSGCQHITSTKCNFSSVELENVFEKIELRIRAEEGNNTSTWYEVEPFVPFLEAQIGPPDVHLEAEDKAIILSISPPGTKDSIMWAMDRSSFRYSVVIWKNSSSLEERTETVYPEDKIYKLSPEITYCLKVKAELRLQSRVGCYSPVYCINTTERHKVPSPENIQINADNQIYVLKWDYPYENATFQAQWLRAFFKKIPGNHSDKWKQIPNCENVTSTHCVFPREVSSRGIYYVRVRASNGNGTSFWSEEKEFNTEMKTIIFPPVISVKSVTDDSLHVSVGASEESENMSVNQLYPLIYEVIFWENTSNAERKVLEKRTNFIFPDLKPLTVYCVKARALIENDRRNKGSSVSDTVCEKTKPGNTSKTWLIVGTCTALFSIPVVIYVVSVFLRCVKYVFFPSSKPPSSVDEYFSDQPLRNLLLSTSEEQTERCFIIENASIITEIEETDEIDEVHKKYSSQTSQDSGNYSNEDENSGSKISEEFPQQDSV

SEQ ID NO: 31 - protein, 179 aa, Homo sapiens
TLLLGWLLAQVAGAAGTTEKAYNLTWKSTNFKTILEWEPKPINHVYTVQISTRSGNWKNKCFYTTDTECDLTDEIVKDVTQTYLARVLSYPARNDQTTGSGEEPPFTNSPEFTPYLDTNLGQPTIQSFEQVGTKLNVTVQDARTLVRRNGTFLSLRDVFGKDLNYTLYYWKASSTGKKT

SEQ ID NO: 32 - protein, 116 aa, Homo sapiens
LPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSIS

SEQ ID NO: 33 - protein, 198 aa, Mus musculus
VLPSAAGGENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYS

(19) United States
(12) Patent Application Publication
(10) Pub. No.: US 2003/0180752 A1
(43) Pub. Date: Sep. 25, 2003

(54) Type 2 cytokine receptor and nucleic acids encoding same

(76) Inventors: Wei Liu, Sudbury, MA (US); Lynette Fouser, Acton, MA (US); Vikki Spaulding, Lowell, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/293,832

(22) Filed: Nov. 12, 2002

Related U.S. Application Data
(60) Provisional application No. 60/332,366, filed on Nov. 9, 2001.

(51) Int. Cl. C12Q 1/68; G01N 33/53; A61K 38/17; C12P 21/02; C12N 5/06; C07H 21/04; C07K 16/28; C07K 14/715

(52) U.S. Cl. 435/6; 435/69.1; 435/320.1; 435/325; 530/350; 536/23.5; 514/12; 530/388.22; 435/7.1

(57) ABSTRACT

The present invention provides novel isolated CRF2-13 polynucleotides and polypeptides encoded by the CRF2-13 polynucleotides. Also provided are the antibodies that immunospecifically bind to a CRF2-13 polypeptide or any derivative (including fusion derivative), variant, mutant or fragment of the CRF2-13 polypeptide, polynucleotide or antibody. The invention additionally provides methods in which the CRF2-13 polypeptide, polynucleotide and antibody are utilized in the detection and treatment of a broad range of pathological states, as well as to other uses.

### RELATED APPLICATION

This application claims priority to U.S. S No. 60/332,366, filed Nov. 9, 2001. The contents of this application are incorporated herein by reference in their entirety.

### FIELD OF THE INVENTION

The invention relates generally to nucleic acids and polypeptides and more specifically to nucleic acids and polypeptides encoding type II cytokine receptors, as well as vectors, host cells, antibodies and recombinant methods for producing the polypeptides and polynucleotides.

### BACKGROUND OF THE INVENTION

Cytokines such as interferons are soluble proteins that influence the growth and differentiation of many cell types. Cytokines exert their effects through cytokine receptors, which are located on the surface of cells responsive to the effects of cytokines. Cytokine receptors are composed of one or more integral membrane proteins that bind the cytokine with high affinity and transduce this binding event to the cell through the cytoplasmic portions of the receptor subunits.

Cytokine receptors have been grouped into several classes on the basis of similarities in their extracellular ligand binding domains. For example, the receptor chains responsible for binding and/or transducing the effect of interferons cytokine are members of the type II cytokine receptor family (CRF2), based upon the presence of a characteristic 200-250 residue extracellular domain.

Members of the CRF2 family have been reported to act as receptors for a variety of cytokines, including interferon alpha, interferon beta, interferon gamma, IL-10, IL-20, and IL-22. Recently identified members of the CRF2 family are candidate ligands for the IL-10-like molecules IL-19, AK155 and mda-7.

The demonstrated in vivo activities of these interferons illustrate the clinical potential of, and need for, other cytokines, cytokine agonists, and cytokine antagonists.

### SUMMARY OF THE INVENTION

The invention is based, in part, upon the discovery of polynucleotide sequences encoding CRF2-13, novel member of the CRF2 family.

Accordingly, in one aspect, the invention provides an isolated nucleic acid molecule that includes the sequence of SEQ ID NO:1, or a fragment, homolog, analog or derivative thereof. The nucleic acid can include, e.g., a nucleic acid sequence encoding a polypeptide at least 70%, e.g., 80%, 85%, 90%, 95%, 98%, or even 99% or more identical to a polypeptide that includes the amino acid sequences of SEQ ID NO:2. The nucleic acid can be, e.g., a genomic DNA fragment, or a cDNA molecule.

Preferably, the isolated nucleic acid molecule encodes a polypeptide comprising an amino acid sequence at least 85% identical to amino acids 21-520 of SEQ ID NO:2. More preferably, the encoded polypeptide is at least 90%, 95%, 98%, 99% identical to amino acids 21-520 of SEQ ID NO:2. In some embodiments, the encoded polypeptide includes amino acids 21-520 of SEQ ID NO:2. For example, the encoded polypeptide in some embodiments includes amino acids 1-520 of SEQ ID NO:2. An example of such an isolated nucleotide is a nucleic acid molecule that includes nucleotides 1-1563 of SEQ ID NO:1.

The invention additionally includes a vector comprising the isolated nucleic acid molecule that encodes a polypeptide comprising an amino acid sequence at least 85% identical to amino acids 21-520 of SEQ ID NO:2, as well as a cell that includes this vector. Also within the invention is a pharmaceutical composition that includes the isolated nucleic acid molecule encoding a polypeptide comprising an amino acid sequence at least 85% identical to amino acids 21-520 of SEQ ID NO:2, along with a pharmaceutically acceptable carrier.

In some embodiments, the nucleic acid molecule encodes a polypeptide with an amino acid sequence having one or more substitutions relative to the amino acid sequence of amino acids 21-520 of SEQ ID NO:2. In some embodiments, the nucleic acid molecule hybridizes under stringent conditions to a nucleic acid sequence complementary to a nucleic acid molecule comprising SEQ ID NO:1. In addition, or in the alternative, the encoded polypeptide binds specifically to a polypeptide ligand.

Also within the invention is are isolated nucleic acids encoding a polypeptide of at least 499 amino acids, wherein the nucleic acid hybridizes under low stringency, moderate stringency, and/or high stringency conditions to SEQ ID NO:1.

In another aspect, the invention provides a substantially purified polypeptide that includes an amino acid sequence at least 85% identical to the amino acid sequence of amino acids 21-520 of SEQ ID NO:2. In some embodiments, the polypeptide is at least 90%, 95%, 97%, 98% or 99% or more identical to the amino acid sequence of SEQ ID NO:1. In some embodiments, the polypeptide differs by one or more substitutions from amino acids 21-520 of SEQ ID NO:2. In other embodiments, the polypeptide includes amino acids 21-520 of SEQ ID NO:2.

Also within the invention is a pharmaceutical composition that includes an amino acid sequence at least 85% identical to the amino acid sequence of amino acids 21-520 of SEQ ID NO:2, and a pharmaceutically acceptable carrier.

Also within the invention is a polypeptide at least 85% identical to amino acids 21-230 of SEQ ID NO:2. For example, the polypeptide can be at least 95%, 97%, 98%, or 99% or more identical to amino acids 21-230 of SEQ ID NO:2. In some embodiments, the polypeptide differs by one or more substitutions from amino acids 21-230 of SEQ ID NO:2. In other embodiments, the polypeptide includes amino acids 21-230 of SEQ ID NO:2.

Also provided by the invention is a fusion polypeptide comprising a CRF2-13 polypeptide operably linked to a non-CRF2-13 polypeptide. In some embodiments, the CRF2-13 polypeptide includes amino acids 21-520 of SEQ ID NO:2. For example, the CRF2-13 polypeptide can includes amino acids 21-230 of SEQ ID NO:2. In some embodiments, the CRF2-13 is at least 499 amino acids in length and is encoded by a nucleic acid that hybridizes under low, moderate, and/or high stringency conditions to SEQ ID NO:1.

The non-CRF2-13 polypeptide can include, e.g., an Fc region of an immunoglobulin molecules or a FLAG epitope, a HIS tag, and a MYC tag.

Also within the invention is a pharmaceutical composition that includes a fusion polypeptide with CRF2-13 polypeptide operably linked to a non-CRF2-13 polypeptide, along a pharmaceutically acceptable carrier.

Also provided by the invention is an antibody that binds to a polypeptide that includes a CRF2-13 polypeptide sequence (e.g., some or all of the amino acid sequence of SEQ ID NO:2). In some embodiments, the antibody neutralizes binding of a CRF2-13 polypeptide to a CRF2-13 ligand. The antibody can be, e.g., a polyclonal antibody or a monoclonal antibody. The monoclonal antibody can be, e.g, a murine monoclonal antibody, or a humanized monoclonal antibody.

Also provided by the invention is a kit comprising in one or more containers a compound selected from the group consisting of an CRF2-13 nucleic acid, an CRF2-13 polypeptide and an antibody to an CRF2-13 polypeptide. The kit may optionally include directions for use. In some embodiments the compound is provided with a pharmaceutically acceptable carrier.

Also provided by the invention is a method of producing a CRF2-13 polypeptide, culturing a cell including a nucleic acid encoding a CRF2-13 polypeptide under conditions allowing for expression of a polypeptide encoded by the nucleic acid.

In a further aspect the invention provides a method of detecting the presence of a CRF2-13 nucleic acid molecule in a biological sample. The method includes contacting the sample with a nucleic acid probe; and identifying the bound probe, if present, thereby detecting the presence of CRF2-13 nucleic acid molecule in the sample.

In some embodiments, the CRF2-13 nucleic acid molecule is detected in a PCR reaction using primers (GCTGCAGGCCGCTCCAGGGAGGCCCCG; (SEQ ID:23) and (CCAGGTATTCGGACTCCACCCAGGGGGAC (SEQ ID NO:24).

Also provided by the invention is a method of detecting the presence of a CRF2-13 polypeptide in a sample by contacting the sample with a compound that selectively binds to the CRF2-13 polypeptide under conditions allowing for formation of a complex between the polypeptide and the compound and detecting the complex, if present, thereby identifying the polypeptide in the sample.

In another aspect, the invention includes a method of modulating the activity of a CRF2-13 polypeptide by contacting a cell sample comprising the polypeptide with a compound that binds to the polypeptide in an amount sufficient to modulate the activity of the polypeptide. The compound can be, e.g., a soluble CRF2-13 polypeptide inhibitor. In some embodiments, the soluble CRF2-13 inhibitor includes a polypeptide at least 85% homologous to amino acids 21-260 of SEQ ID NO:2.

Also provided by the invention is a method for screening for a modulator of activity or of latency or predisposition to a cytokine-mediated immune disorder. The method includes contacting a test compound with a CRF2-13 polypeptide; and determining if the test compound binds to the CRF2-13 polypeptide. Binding of the test compound to the polypeptide indicates the test compound is a modulator of activity or of latency or predisposition to a cytokine-mediated immune disorder.

In another aspect, the invention provides a method for screening for a modulator of activity or of latency or predisposition to a cytokine-mediated immune disorder. The method includes administering a test compound to a test animal suffering from or at increased risk for the immune disorder, wherein the test animal recombinantly expresses a CRF2-13 and measuring expression of the activity of the polypeptide in the test animal. The activity of the polypeptide is also measured in a control animal that recombinantly expresses the polypeptide and is not at increased risk for the immune disorder. The expression of the polypeptide in the test animal and the control animal is compared. A change in the activity of the polypeptide in the test animal relative to the control animal indicates the test compound is a modulator of latency of the immune disorder. The cytokine-mediated immune disorder can be, e.g., an autoimmune disorder, a T-lymphocyte-associated disorder, a cell-proliferation disorder, a cell differentiation disorder, or an immune deficiency order.

Also provided by the invention is a method for determining the presence of or predisposition to a disease associated with altered levels of a CRF2-13 polypeptide in a subject (such as a human). The method includes measuring the amount of the polypeptide in a sample from the subject; and comparing the amount of the polypeptide to the amount of the polypeptide present in a control sample. An alteration in the level of the polypeptide in the subject sample as compared to the level of the polypeptide in the control sample indicates the presence of or predisposition to a disease in the subject.

Also provided by the invention is a method for determining the presence of or predisposition to a disease associated with altered levels of a CRF2-13 nucleic acid molecule in a subject (such as a human). The method includes measuring the amount of the nucleic acid in a sample from the subject; and comparing the amount of the nucleic acid in the subject sample to the amount of the nucleic acid present in a control sample. An alteration in the level of the nucleic acid in step (a) as compared to the level of the nucleic acid in the control sample indicates the presence of or predisposition to the disease in the subject.

The invention also provides a method of treating or preventing a pathological condition associated with a cytokine-mediated disorder by administering to a subject (such as a human) an agent that increases levels of a polypeptide comprising the extracellular amino acid sequence of a CRF2-13 polypeptide in an amount sufficient to alleviate or prevent the pathological condition in the subject. In some embodiments the agent is a polypeptide that includes the extracellular amino acid sequence of a CRF2-13 polypeptide. For example, the polypeptide can be a fusion polypeptide comprising the extracellular amino acid sequence of a CRF2-13 polypeptide fused to a non-CRF2-13 polypeptide). In other embodiments, the agent is a nucleic acid encodes a polypeptide that includes the extracellular amino acid sequence of a CRF2-13 polypeptide.

Also provided by the invention is a method of treating or preventing a pathological condition in a subject by administering to the subject an antibody that binds specifically to a CRF2-13 polypeptide in an amount sufficient to alleviate or prevent the pathological condition. The subject can be, e.g., a human.

Also provided by the invention is a method of treating rheumatoid arthritis in a subject, the method comprising administering to the subject an agent that modulates the amount of a CRF2-13 polypeptide in the subject. The subject can be, e.g., a human. In some embodiments, the agent increases the amount of the CRF2-13 polypeptide in the subject. The agent can be, e.g., a CRF2-13 nucleic acid or polypeptide. In other embodiments, the agent decreases the amount of the CRF2-13 polypeptide in the subject. The agent is an anti-CRF2-13 antibody.

Also within the invention is a method of treating multiple sclerosis in a subject by administering to the subject an agent that modulates the amount of a CRF2-13 polypeptide in the subject.

The invention additionally provides a method of modulating vascular smooth muscle cell proliferation, the method comprising contacting a vascular smooth muscle cell with an agent that modulates the amount of CRF2-13 polypeptide in the cell.

In a further aspect, the invention includes a method of treating or preventing inflammation in a subject, the method comprising administering to the subject an agent that modulates the amount of a CRF2-13 polypeptide in the subject.

Also provided by the invention are polymorphic CRF2-13 sequences containing one or more alterations in sequence relative to the nucleotide sequence disclosed in SEQ ID NO:3.

For example, the invention includes an isolated polynucleotide comprising at least 10 contiguous nucleotides from nucleotide 30957 to nucleotide 30967 of SEQ ID NO:3, provided that position 30962 of the polynucleotide is “A or “G”. In some embodiments, the isolated polynucleotide includes at least 15 or at least 20 contiguous nucleotides. In some embodiments, the polynucleotide is between about 10 and about 100 nucleotides in length, e.g., between about 10 and about 90 nucleotides in length, between about 10 and about 75 nucleotides in length, between about 10 and about 50 bases in length, or between about 10 and about 40 bases in length.

The invention also includes an isolated polynucleotide comprising at least 10 contiguous nucleotides from nucleotide 30650 to nucleotide 30660 of SEQ ID NO:3, provided that position 30655 of the polynucleotide is “A” or “G”.

In another aspect, the invention includes an isolated polynucleotide comprising at least 10 contiguous nucleotides from nucleotide 28739 to nucleotide 28749 of SEQ ID NO:3, wherein position 28744 of the polynucleotide is “A” or “G”.

In a further aspect, the invention includes an isolated polynucleotide comprising at least 10 contiguous nucleotides from nucleotide 28442 to 28452 of SEQ ID NO:3, wherein position 28448 of the polynucleotide is “C” or “T”.

In a still further aspect, the invention includes an isolated polynucleotide comprising at least 10 contiguous nucleotides from nucleotide 9421 to 9431 of SEQ ID NO:3, wherein position 9426 of the polynucleotide is “A” or “G”.

In yet another aspect, the invention includes an isolated polynucleotide comprising at least 10 contiguous nucleotides from nucleotide 9157 to 9167 of SEQ ID NO:3, wherein position 9162 of the polynucleotide is “A” or “G”.

In a further aspect, the invention includes an isolated polynucleotide comprising at least 10 contiguous nucleotides from nucleotide 8806 to 8816 of SEQ ID NO:3, wherein position 8811 of the polynucleotide is “C or “T”.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a phylogram showing polypeptide sequences related to a CRF2-13 polypeptide according to the invention.

### DETAILED DESCRIPTION OF THE INVENTION

The invention is based in part on the discovery of novel nucleic acid sequences encoding a polypeptide showing homology to CRF2 polypeptides. Included in the invention is a 1563 nucleotide sequence (SEQ ID NO:1) shown in Table 1. Nucleotides 1-1560 of SEQ ID NO:1 encode a 520 amino acid CRF2-like polypeptide. The amino acid sequences of the encoded polypeptide is shown in Table 2 (SEQ ID NO:2). A nucleic acid having a portion of the 5′ untranslated region and a portion of the coding sequence shown in Table 1 was identified in a human placental cDNA library.

TABLE 1

(SEQ ID NO:1)

ATGGCGGGGCCCGAGCGCTGGGGCCCCCTGCTCCTGTGCCTGCTGCAGGCCGCTCCAGGGAGGCCCCGTCTGGCCCCT
CCCCAGAATGTGACGCTGCTCTCCCAGAACTTCAGCGTGTACCTGACATGGCTCCCAGGGCTTGGCAACCCCCAGGAT
GTGACCTATTTTGTGGCCTATCAGAGCTCTCCCACCCGTAGACGGTGGCGCGAAGTGGAAGAGTGTGCGGGAACCAAG
GAGCTGCTATGTTCTATGATGTGCCTGAAGAAACAGGACCTGTACAACAAGTTCAAGGGACGCGTGCGGACGGTTTCT
CCCAGCTCCAAGTCCCCCTGGGTGGAGTCCGAATACCTGGATTACCTTTTTGAAGTGGAGCCGGCCCCACCTGTCCTG
GTGCTCACCCAGACGGAGGAGATCCTGAGTGCCAATGCCACGTACCAGCTGCCCCCCTGCATGCCCCCACTGGATCTG
AAGTATGAGGTGGCATTCTGGAAGGAGGGGGCCGGAAACAAGACCCTATTTCCAGTCACTCCCCATGGCCAGCCAGTC
CAGATCACTCTCCAGCCAGCTGCCAGCGAACACCACTGCCTCAGTGCCAGAACCATCTACACGTTCAGTGTCCCGAAA
TACAGCAAGTTCTCTAAGCCCACCTGCTTCTTGCTGGAGGTCCCAGAAGCCAACTGGGCTTTCCTGGTGCTGCCATCG
CTTCTGATACTGCTGTTAGTAATTGCCGCAGGGGGTGTGATCTGGAAGACCCTCATGGGGAACCCCTGGTTTCAGCGG
GCAAAGATGCCACGGGCCCTGGACTTTTCTGGACACACACACCCTGTGGCAACCTTTCAGCCCAGCAGACCAGAGTCC
GTGAATGACTTGTTCCTCTGTCCCCAAAAGGAACTGACCAGAGGGGTCAGGCCGACGCCTCGAGTCAGGGCCCCAGCC
ACCCAACAGACAAGATGGAAGAAGGACCTTGCAGAGGACGAAGAGGAGGAGGATGAGGAGGACACAGAAGATGGCGTC
AGCTTCCAGCCCTACATTGAACCACCTTCTTTCCTGGGGCAAGAGCACCAGGCTCCAGGGCACTCGGAGGCTGGTGGG
GTGGACTCAGGGAGGCCCAGGGCTCCTCTGGTCCCAAGCGAAGGCTCCTCTGCTTGGGATTCTTCAGACAGAAGCTGG
GCCAGCACTGTGGACTCCTCCTGGGACAGGGCTGGGTCCTCTGGCTATTTGGCTGAGAAGGGGCCAGGCCAAGGGCCG
GGTGGGGATGGGCACCAAGAATCTCTCCCACCACCTGAATTCTCCAAGGACTCGGGTTTCCTGGAAGAGCTCCCAGAA
GATAACCTCTCCTCCTGGGCCACCTGGGGCACCTTACCACCGGAGCCGAATCTGGTCCCTGGGGGACCCCCAGTTTCT
CTTCAGACACTGACCTTCTGCTGGGAAAGCAGCCCTGAGGAGGAAGAGGAGGCGAGGGAATCAGAAATTGAGGACAGC
GATGCGGGCAGCTGGGGGGCTGAGAGCACCCAGAGGACCGAGGACAGGGGCCGGACATTGGGGCATTACATGGCCAGG
TGA

TABLE 2

(SEQ ID NO:2)

MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSGRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

The nucleic acid of Table 1 encodes the 520 amino acid sequence (SEQ ID NO:2) shown in Table 2. Signal P and Psort results predict that CRF2-13 protein contains a signal peptide, and is likely to be localized to the plasma membrane with a certainty of 0.460. The most likely cleavage site for a CRF2-13 polypeptide is between amino acids 246 and 247, at: AGG-VI.

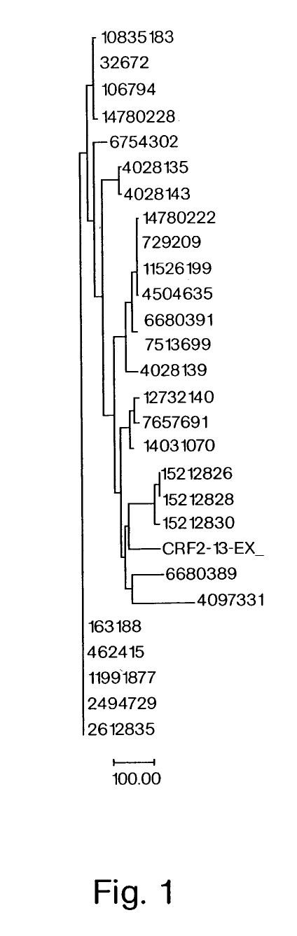

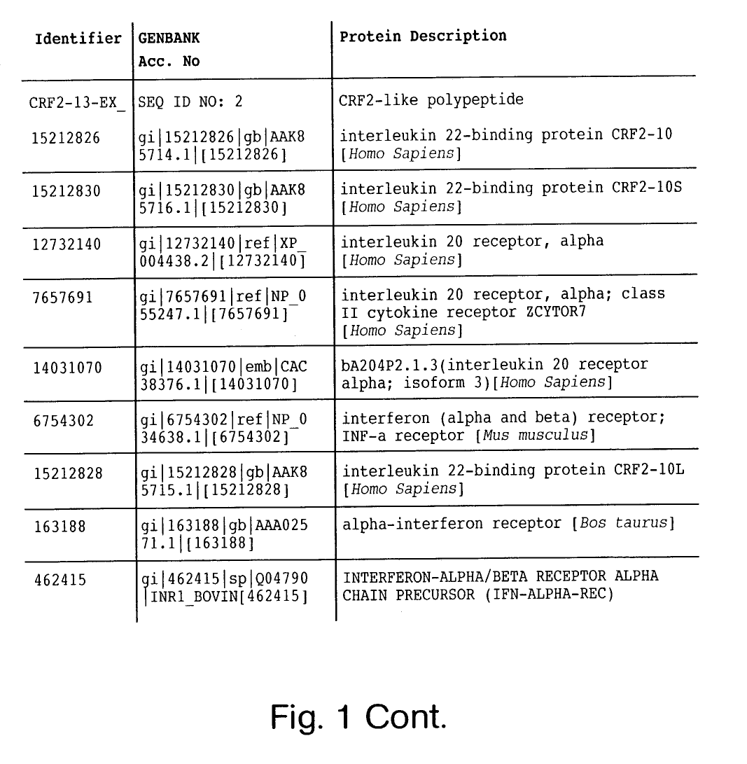

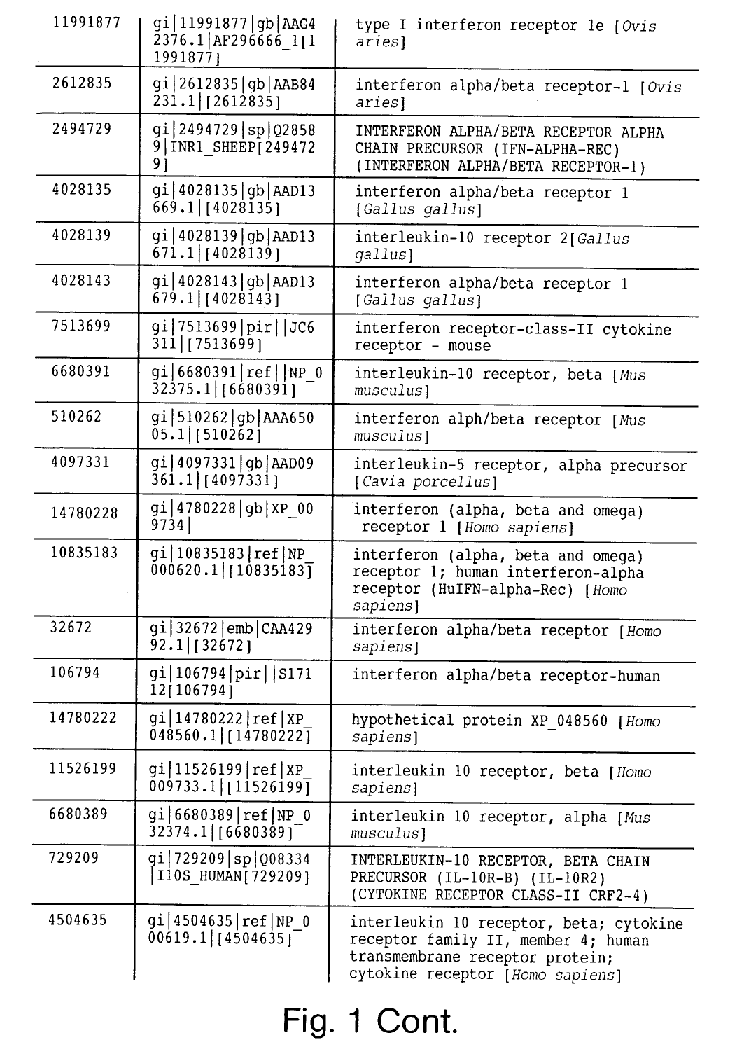

The CRF2-13 amino acid sequence is related to other previously described interleukin-binding proteins. The relationship is schematically represented in FIG. 1. The CRF2-13 amino acid sequence of SEQ ID NO:2 has 40 of 111 amino acid residues (36%) identical to, and 56 of 111 (50%) amino acid residues similar to, the 231 amino acid residue human interleukin 22-binding protein CRF2-10 (gi|15212826|). Similarly, the CRF2-13 amino acid sequence has 32 of 86 amino acid residues (37%) identical to, and 43 of 86 (49%) amino acid residues similar to, the 130 amino acid residue human interleukin 22-binding protein CRF2-10S (gi|15212830|). Moreover, the CRF2-13 amino acid sequence has 41 of 142 amino acid residues (28%) identical to, and 58 of 142 (39%) amino acid residues similar to, the 130 amino acid residue human interleukin 22-binding protein CRF2-10L (gi|15212828|).

CRF2-13 polypeptide also shows homology to the amino acid sequences shown in the BLASTP data listed in Table 3A. Homologies are calculated according to the method of Altschul and coworkers (Nucleic Acids Res. 25:3389-3402, 1997).

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the IIT BLAST analysis, matched the Query IIT sequence purely by chance is the E value. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences. Blasting is performed against public nucleotide databases such as GenBank databases and the GeneSeq patent database. For example, BLASTX searching is performed against public protein databases, which include GenBank databases, SwissProt, PDB and PIR.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/.

TABLE 3A
BLAST results for NOV10

| Gene Index/ | Protein/ | Length | Identity | Posit- | |
|---|---|---|---|---|---|
| Identifier | Organism | (aa) | (%) | ives (%) | Expect |
| gi\|15212826\|gb | interleukin | 231 | 40/111 | 56/111 | 2e−08 |
| \|AAK85714.1\| | 22-binding | | (36%) | (50%) | |
| (AY040566) | protein CRF2- | | | | |
| | 10 [Homo | | | | |
| | sapiens] | | | | |
| gi\|15212830\|gb | interleukin | 130 | 32/86 | 43/86 | 2e−05 |
| \|AAK85716.1\| | 22-binding | | (37%) | (49%) | |
| (AY040568) | protein CRF2- | | | | |
| | 10S [Homo | | | | |
| | sapiens] | | | | |
| gi\|15212828\|gb | interleukin | 263 | 41/142 | 58/142 | 3e−05 |
| \|AAK85715.1\| | 22-binding | | (28%) | (39%) | |
| (AY040567) | protein CRF2- | | | | |
| | 10L [Homo | | | | |
| | sapiens] | | | | |
| gi\|432\|emb\|CAA | interferon | 560 | 40/170 | 75/170 | 0.001 |
| 48484.1\| | receptor type | | (23%) | (43%) | |
| (X68443) | 1 [Bos | | | | |
| | taurus] | | | | |
| gi\|163188\|gb\|A | alpha- | 560 | 0/170 | 75/170 | 0.001 |
| AA02571.1\| | interferon | | (23%) | (43%) | |
| (L06320) | receptor [Bos | | | | |
| | taurus] | | | | |

The homology of these sequences are graphically depicted in the ClustalW analysis of Table 3B.

[image]

[image]

[image]

The presence of identifiable domains in the protein disclosed herein was determined by searches using algorithms such as PROSITE, Blocks, Pfam, ProDomain, Prints and then determining the ProDom or Interpro number by crossing the domain match (or numbers) using either the Interpro website (http:www.ebi.ac.uk/interpro/) or the ProDom database (http://www.biochem.ucl.ac.uk/bsm/dbbrowser/jj/prodomsrchjj.html). Tables 3C-3E list the domain descriptions from DOMAIN analysis results of CRF2-13 polypeptide using Pfam (Table 3C) and ProDomain (Tables 3D and 3E). This indicates that the CRF2-13 protein sequence has properties similar to those of other proteins known to contain these domains.

TABLE 3C
Domain Analysis of CRF2-13 Protein

gnl|Pfam|pfam011O8 Tissue_fac, Tissue factor (SEQ ID NO: XX) CD-
Length = 293 residues,61.1% aligned Score = 37.0 bits (84),
Expect = 0.003

| | | | |
|---|---|---|---|
| Query: | 9 | PLLL--CLLQAAPGRPRLAPPQNVTLLSQNFSVYLTWLPGLGNPQDVTYFVAYQSSPTRR | 66 |
| | | \|\|\| \| \| \| \|+\| \| \|\| \| \| \| \| + \| \| \| \| | |
| Sbjct: | 19 | TLLLGWLLAQVAGAAGTTEKAYNLTWKSTNFKTILEWEP---KPINHVYTV--QISTRSG | 73 |
| Query: | 67 | RWREVEECAGTKELLCSMMCLKKQDLYNKFKGRV--------RTVSPSSKSPWVES-EYL | 117 |
| | | \|+ +\| \| + \| + +\|+ + \|\| +\| + \|+ \| \|+ | |
| Sbjct: | 74 | NWK--NKCFYTTDTECDLTDEIVKDVTQTYLARVLSYPARNDQTTGSGEEPPFTNSPEFT | 131 |
| Query: | 118 | DYL-----------FEVEPAPPVLVLTQTEEILSANATYQLPPCMPPLDLKYEVAFWK-E | 165 |
| | | \|\| \|\| + + ++ \| \|+ + \|\| \| + +\|\| | |
| Sbjct: | 132 | PYLDTNLGQPTIQSFEQVGTKLNVTVQDARTLVRRNGTFLSLRDVFGKDLNYTLYYWKAS | 191 |
| Query: | 166 | GAGNKT 171 | |
| | | \| \|\| | |
| Sbjct: | 192 | STGKKT 197 | |

TABLE 3D
Domain Analysis of CRF2-13 Protein

PD338678 (Q9UHF4_HUMAN 36-246)COAGULATION FACTOR III PALMITATE
TISSUE LIPOPROTEIN SIGNAL GLYCOPROTEIN TRANSMEMBRANE PRECURSOR
(SEQ ID NO: XX)Score = 101 (43.3 bits), Expect = 0.003
Identities = 33/118 (27%) , Positives = 50/118 (41%)

| | | | |
|---|---|---|---|
| Query: | 24 | LAPPQNVTLLSQNFSVYLTWLPGLG-NPQDVTYFVAYQSSPTRRRWREVEECAGTKELLC | 82 |
| | | \| \| \|+\| \|\| \| \| \| \| \| \|\|\| \| \| +++\| \|\| \| | |
| Sbjct: | 37 | LPKPANITFLSINMKNVLQWTPPEGLQGVKVTYTVQYFIY-GQKKWLNKSECRNINRTYC | 95 |
| Query: | 83 | SMMCLKKQDLYNKFKGRVRTVSPSSKSPWVESEYLDYLFEVEPAPPVLVLTQTEEILS | 140 |
| | | + + \| +++ +\|+ + + \| \| \|\| \| + \|\| + \|\| \|+ +\| | |
| Sbjct: | 96 | DLSA-ETSDYEHQYYAKVKAIWGTKCSKWAESGRFYPFLETQIGPPEVALTTDEKSIS | 152 |

TABLE 3E
Domain Analysis of CRF2-13 Protein

PD008555 (INR1_MOUSE 19-216)RECEPTOR TRANSMEMBRANE GLYCOPROTEIN
PRECURSOR CHAIN SIGNALINTERFERON-ALPHA/BETA IFN-ALPHA-REC (SEQ
ID NO: XX)Score = 98 (42.1 bits), Expect = 0.007 Identities =
46/207 (22%), Positives = 88/207 (42%)

| | | | |
|---|---|---|---|
| Query: | 14 | LLQAAPGRPRLAPPQNVTLLSQNFSVYLTWLPGLGNPQDVTYFVAYQSSPTRRRWREVEE | 73 |
| | | +\| +\| \| \| \|\|+\|+ + + + \| \| + \|\|+ \|++ +\| +\| \| | |
| Sbjct: | 19 | VLPSAAGGENLKPPENIDVYIIDDNYTLKWSSHGESMGSVTFSAEYRTK-DEAKWLKVPE | 77 |
| Query: | 74 | CAGTKELLCSMMCLKKQDLYNKFKGRVRTVSPSSKSPWVESEYLDYLFEVEPAPPVLVLT | 33 |
| | | \| \| \| \| ++\| \| + \|\|\| +\| \| \| \| + + +\|\| + \| | |
| Sbjct: | 78 | CQHTTTTKCEFSLLDT-NVYIKTQFRVRAEEGNSTSSWNEVDPFIPFYTAHMSPPEVRLE | 36 |
| Query: | 134 | QTEEILSANATYQLPP-------CMPPLDLKYEVAFWKEGAGNKTLFPVTPHGQPVQITL | 86 |
| | | ++ + + + \|\| + \| + \|++ + +\| \| + + + \| | |
| Sbjct: | 137 | AEDKAILVHIS---PPGQDGNMWALEKPSFSYTIRIWQKSSSDKKTINSTYYVEKIP-EL | 192 |
| Query; | 187 | QPAASEHHCLSARTIYTFSVPKYSKFS 213 | |
| | | \| + +\|\| + \|+ \|+ \|+\| +\| | |
| Sbjct: | 193 | LPETT--YCLEVKAIHP-SLKKHSNYS 216 | |

Growth factors such are proteins that bind to receptors on the cell surface, with the primary result of activating cellular proliferation and/or differentiation. Cytokines (e.g., lymphokines; interleukin and interferon) are a unique family of growth factors. A number of receptors for lymphokines, hematopoeitic growth factors and growth hormone-related molecules share common domains, and can be divided into families. The cytokine receptor class 2 family includes interleukin-10 receptor; interferon-gamma receptor; interferon-alpha/beta receptor; and tissue factor (Konigsberg et al., Nature 380:41-46, 1996). The presence of regions of CRF2-13 polypeptide related to domains found on tissue factor and coagulation factor III palmitate tissue lipoprotein signal glycoprotein transmembrane precursor are consistent with the localization of CRF2-13 polypeptide to the plasma membrane and the assignment of CRF2-13 polypeptide to the cytokine receptor superfamily. The presence of a region of CRF2-13 polypeptide related to interferon 1 receptor transmembrane glycoprotein precursor signal chain interferon alphalbeta IFN-alpha receptor reinforces this assignment.

The nucleotide sequence shown in Table 1 was identified as part of the genomic DNA sequence shown in Table 4:

TABLE 4

| | | |
|---|---|---|
| 1 | GAAAGAGAGA GAAAAAAGAA GGAAGGAAGG AAGGAAGGAA GGAAGGAAGG | (SEQ ID NO:3) |
| 51 | AAGGAAAGAA AGAAAGAAAG AAAGAAAGAA AGAAAGAAAG AAAGAAAGAA | |
| 101 | AGAGAGAGAA AGGAAGGAAG GAAGGAGAAA AGAAAGTCAA CAGTCAACAT | |
| 151 | TTCAGAGATC CCAAGATACC AACACTGACC GTGCCTGCTG CTCTTCCATC | |
| 201 | CTCCTCCACC CTGCGCCTTT GAGGTGGAAT TGCGTCCTCT GTGAGCAGGG | |
| 251 | CTTTGTTAAG AGATCCTAAT TAAGGCCAGG CACAGTGGCT CATGCCTGTA | |
| 301 | ATCCCAGCAC TTTGGGAGGC TGAGGTCACC TGAGGTCAGG AGTTCAAGAC | |
| 351 | CAGCCTGCCC AACATGGTGA AACCCCATCT CTACAAAAAT TAGCTGAGCA | |
| 401 | TGATGGCAGG TGCCTGTAAT CCCAACTACT TGGGAGGCTG AAGTGAGAAA | |
| 451 | ATAGCTTGAA CCCAGGAGGC GGGGTTGCAG TGAGCCAAGA TCACACTATT | |
| 501 | GCATTCCAGC CTGGGCGACA GAGCTTTTGT CTAAAAAAAA AAAAAGAAAA | |
| 551 | AAAATCCTGA TTAAGCAGAA GCCTTGATGC TAGTCCCAGA AGCATCCTGA | |
| 601 | AATTTCCAAA AGAAATTTCC CCCGCGGTTA AACTCAGAGC AACTTTTGGA | |
| 651 | CCCACCAAGC TCTGTGAAAA TCATTTTCTC TTCCAAAAAC TGATGGGACC | |
| 701 | AAAGCTGATC CCAGTTTCAA ATAATTATCA AAAAATTGGA AACGAAATAT | |
| 751 | GATCAGAAAA GAAGAAAGTT GAAAAAGAAA ATCCTTATCA CCCAAAGACA | |
| 801 | ACAACCATTA ATATTTTGGT AATTATTATT ACAAATATCT TTCTATGCAT | |
| 851 | ACAGACAGAC TCACACACAC ACACACACAC ACACACACAC ACTTTTTTTT | |
| 901 | TTTTTTTTGA AACTGAGTTT CACTCTGTCG CCCAGGCTGG AGTGCAGTGG | |
| 951 | CGCGATCTCG GCTCACTGCA ACCTCCGCCT CCTGGGTTCA AGCGATTCTC | |
| 1001 | CTGCCTCAGC CTCCCTGATA GCTGGGATTA CAGGTGAATG CCACCACGCC | |
| 1051 | CGGCTGATTT TCTGTATTTT TAGTAGAGAC GGGGTTTCAC CATGTTGGCC | |
| 1101 | AGGCTTGTCT CCAACTCCTG ACCTCAGGCG ATCCACCCGC CTCACCCTCC | |
| 1151 | CAAAGTGCTG GGATTACAGG CGTGAGCCAC CGCGCCCGGC TACACACACA | |
| 1201 | CTTTTTTAAT GGGCCTATGT TTTAGCACTC GCTTTTCTGT TTCTCAGTGT | |
| 1251 | GTTGCAAACA CCTCGGTGTC GATACACACC ATTCGGCAAC GTCCTCCTAA | |
| 1301 | AGGGCCGCAT AATATTGCGC GTCGTGGCGT GTGCCTTACT GGGAAGCTAC | |
| 1351 | TGCTGTCCAG GTGAACACCA CAGCCTTCGG GGTCAGAAAG ACAGCTTTCC | |
| 1401 | CCAGAACAAG CACCTGAAGC TCTGGGGCCT GCCGCTCCCC GGGTAGAGAA | |
| 1451 | GTACGTGGAG AAGGGCAGCA CGGATCCGCC GGGATCCCCG GGGGCATTAA | |
| 1501 | AGGGAATCGC GTGTGTAAGG CGCGGAGCTC AGCATCCGGC TCAGAAACGC | |
| 1551 | GCTCGGATCC CGCCAATGGC ATTGAGGCCG CGTAGCCAAA CCGGCCTTGA | |
| 1601 | ACTCTCCCTA ATCCTGCCAA AATGGCCCGT CCTGGAGCAC TGGACTGGCC | |
| 1651 | GTGGGTTATT GATCATCAGC CGGTTTCTTC CCCTCCCCTG CCCTTCCCCC | |
| 1701 | GTGCACGGAT TTACTGATTT TTTTTTCCGG GAATTGAGTA AAACAAAACT | |
| 1751 | AAGTGCAGAT GAAGCAGAGG TACGGGCGAG TTTCGAGCGC GGGGACCGGC | |
| 1801 | GCGCTCCCCC CCCCCTCCCC CCGCGGCGGG GCTGTCCCCA GGGACCTTCT | |
| 1851 | CAGTGAATCC TAGGCGGCAG GGACGGGCCC GCGGCTCTGC GGGCCATTGG | |
| 1901 | CTGCCGACTG CGTCACCTGC CCGCGGTGGG CTAGGAGACG GGAGGCGGGA | |
| 1951 | GGCGGGAGGC GGGGACCTGG GTCCGGGCGG GGACGCCGCG GCAGGAAGGC | |
| 2001 | CATGGCGGGG CCCGAGCGCT GGGGCCCCCT GCTCCTGTGC CTGCTGCAGG | |
| 2051 | CCGCTCCAGG TAAGGGCGCG GGGCCGCGGG AGGGAGGGGG AAGAGGGCTC | |
| 2101 | CCCGGGCCGG GCCGCGCCTA CCCTCGGACC CAGAGCTCCT GGGACAGGCA | |
| 2151 | CGGGGTCCGC AGCCACCCGA GCCGGGTGCG AATCGGCCCT GCCTACGCGC | |
| 2201 | CCCCAGTTTG CTTCTTCCCA GGACTGAACA GAACCGGGTC TTTGATATTC | |
| 2251 | CTCTCCCGCA GGAAACGAAT CCAGTTTCCT AATGCTTCCA GCTTCAGGAG | |
| 2301 | AACTGGAGAA AAAAGACAGC GGCAGTTTGA TACTGCATAT TTTTTAATAA | |
| 2351 | AGTGCTTTTT AATGTTTCCT AAAGAAAGCA CTGATCCCTG CGTGAAAACC | |
| 2401 | ACACTTGACC CTAAAGTGTG GACAGCAGGG AAAGTGGGAC CGATTGATGT | |
| 2451 | CCCTTCCCGT TCCTGCCAGG CCTCTGGTGG GACGGAGCTC TGGTCGCCTG | |
| 2501 | TGCCCTGCTT TCTAACAAGA CGGCTTTCTT TTGGTGGTGG TTGTTGTTTT | |
| 2551 | GTTGTTGTTT TGTTGTTGTT GTTGTTGTTG TTGTTTTCCC ACCTCTACTG | |
| 2601 | ATGAGTAAGG TGTCAGGTAC AAAATTCCTC GCCGTAGGAC CCAACCACCA | |
| 2651 | AACCTCACCG CCCACGACTC CAACCGAAGC AGGGAAGAGA AGGTCCAGAA | |
| 2701 | ATCGCCCCCA GGATATTTTC CTAGTCTTGG ACTCACAGTT TAAAGAGCTG | |
| 2751 | TAAAGGTCCC TGGGCATAAT CCAATCATCA TAAAAGCCTA TATTTATTCA | |
| 2801 | GCAACTTCTT TGTGCCAGGC ACCGCATTAT TCTGGAAGCC TCACGACCCA | |
| 2851 | GCCATCCTAG GAGGTAGATA TTATTTTTAC TTTTCCGATG GGAAAACTGA | |
| 2901 | GGCTCAGAGC AATTCAGGGA ATTCCTCAAG AAGGACGGCA GAGGTGAGGC | |
| 2951 | ACACAGAAGA GAGAAGAGGG GCTAAAGCAA GCCTGGCTAG CTTTTGCCTC | |
| 3001 | CAGGGTAGGC ACGTGGGACA GGCTGTCCAT CCACTGGGTC ACTAGGCCAG | |
| 3051 | CCAGGGATGC TCCAGCCCCC AGTGCCCACA GCAGCGTTCT CTGTGGCTGA | |
| 3101 | TGAGGGACCG TGTACCTGTG TGTGGAGGGA GGGTGGGGTC TTCTGTTCCC | |
| 3151 | CTTTCACTGT CAAGCCCAGA CCTTCTTGTA CTTTCACCTG ATAAGTATTT | |
| 3201 | AATATACACA ACACTAACTA TGGTGTGATG ATTTAGGAGT AAGTACAGCC | |
| 3251 | AGATCTAAGT TCAAATACTG GCTCCCACAC AAACTGACTG TGTAGCCTCA | |
| 3301 | GGCAAGTTAG TTAGCATCTG TCTCTGAGCC TAGCGCCCTT TCCATGGAAG | |
| 3351 | CAGAATGAAT GACACCTACC CCATAGGGTG GTCTGTCCCA AGGGTGATTG | |
| 3401 | AGGTTTTACA TGTAAAGAGC CAAACTAGTG CCTGGCATCC TTTGAAGGCT | |
| 3451 | TCATAGAGGA AAGTTGCTCT AGCTGCTGTT TTTCTCATGT GACCTAGCTC | |
| 3501 | GAATCTGGGG ACTGTCCTGC CCATAGGATA CCTTACAAGT GGCTTGCAGA | |
| 3551 | CAGCCTGGTC TCCTGCTGGT CACCCGTTAG GAAGTCCAGA AGCTGGGAGT | |
| 3601 | AGTAATAGCA CTAGCCTCGT GGTGATACAG TCCCAGCTAG AGGACACAGG | |
| 3651 | ATGAGGTGGA AGCAGGCACC CACTTTTGGG TCTAAAAGGT GATGGGTAGG | |
| 3701 | CAGCCGAGGC TGGGGACAGC CATCCACAGA ACTGGACCCT CCCTCCCTGA | |
| 3751 | TGCCATTTTG CAACCCGTAT GGATTTCCAT CATGGCACAT GGGACACTTC | |
| 3801 | AGGACCCTGA ATTCTCCATG GGACCATGAG CTCCTATAGG GCAGGAATGA | |
| 3851 | AGTTGTGTTC TTCTTTGAAA CCCCTGGCAC ACCGTGGTCA ACAGATCTTG | |
| 3901 | TTTGACTCGT AGTGGTCAAT AGATGGAATA GTTGGAATCA TAAAGCTCAA | |
| 3951 | TAGACCCCAT GAGAACCTAG AAGACAAAGT ACAGTCAAGA GCTCGGACTT | |
| 4001 | TGGAGTTGGC TAGGCCTGGA CTGAATCTGA TTCTACAACT TAATAGCTGA | |
| 4051 | GAGGGCCTTG GTTTTCCCAT CTGTAAAGAT TATAATTATT ATAATGAATA | |
| 4101 | CCTACCTCCT AGGGATGTAA TGAGGATTAA AAGAGAAAGT GCAGGTAAAC | |
| 4151 | TGTTTAACAC AGAACCTGGC TCATAGAACA CAATACACAT TAGCTGCTAT | |
| 4201 | TATTATTATT ATTATTTTAT TTATTTATTT TGAGACAGAG TCTCACTCTG | |
| 4251 | TCACCCAGGC TGGAGTGCAG TGGCGCAATC TCGGCTCACT GCAACCTCCA | |
| 4301 | CCTATCGGGT TCAAGCAATT CTCGTGTCTC AGCCTCCCAA GTAGCTGAGA | |
| 4351 | TGACAGGCGT GTGCCACCAT GCCCAACTAA TTTTTGTATT TTTAGAAGAG | |
| 4401 | ACGTGGTTTC ACCATGTTGG CCAGGCTGGT CTCAAACTCC TGACCTCAGG | |
| 4451 | TGATTTGCCT ACCTCTGCCT CCCAAAATGC TGGGATCACA GGGGTGAGTT | |
| 4501 | ACCATGCCCG GCCTTAGCTG CTATTATTAT CATCATCGTT ATCATCATCA | |
| 4551 | TCATCACCTC GTAGATATGT CAAGGAAGAT TCCCTGGAGG AAGTGACATT | |
| 4601 | TGAATCAAGT ATTTCAAAGA CTAGATGGTG AATACCAGGC AGTCAAAGAC | |
| 4651 | ACCTGGGTTT AAAAACATCC AGAAGAATGC AGTGGCTTGG CAACATCGAG | |
| 4701 | CAGGAAGATT GCCTGATGAG CCTGTAGGGT AGCTGTTGGG GAGAGAGCAG | |
| 4751 | CAAGACGGCC TGGCCAGGCC AGGCCAGGCC ACGTCAGGCA GGGCCTCACA | |
| 4801 | AACCTCAATA ACAAATGTGG ACTTTATTCT GAGGCCAAGG AAAGGGCATG | |
| 4851 | AAACTGGGGA GTGGTGTAAT CAGATGCGTA TTTCAGAAGA TGAAGATTAA | |
| 4901 | CAGTGAGAAG GAAAATGTGC CACAGAGGGG AATAGAGGTC AGTTAAAGGG | |
| 4951 | AGTCAGGGAA AGTGTCCTCG AGACAGTGAC ATCAAAGGAA TGTGAAAACA | |
| 5001 | GCAAAGGAGT GAGCCAGGTG GATATCCAGG GGCAGAACTG TTAAGGCAGA | |
| 5051 | GGGAACAGCA TGAGGGAACA GCGTGTGCAA AGGCCTGGAG TTGGGAGTGT | |
| 5101 | GGCTGGGGTG CTCCAGGAAG GGCAAAAAGT CCTGTGTGGA TGGAGATATG | |
| 5151 | GGAGCAAGGG AGGAGTGGTG GGTCAGATTG GGTAGGGCCT TGGTGGTGAT | |
| 5201 | TGTAAAGACT TTGGAGTTTA GACCAGGCAC AGTGGCTCAG GCCTGTAATC | |
| 5251 | CCAGCACTTT GAGAGGCCAA GGTGGGCGGA TCACCTGAGG TCAGGAGTTC | |
| 5301 | GAGACCAGCC TGTAATCCCA GCTACTCTGG AGGCTGAGGC AGGAGAATCG | |
| 5351 | CTTGAACCCG GAAGGTGGAG GTTGCAGTGA GCTGAGATTG TGCCACTGTA | |
| 5401 | CTCCAGCCTG GGTGGCAGCA TAAGACTCTG CCTCAAAATA AAATAAAAAT | |
| 5451 | AATAAAGACT TTTGAGTTTC CCTGGAGTGA GAGGAAAGCC TTAGAGGGCT | |
| 5501 | TTAGCAGAAG ATGAACATGA TCTGATTTTC ATTTTTAATC CTTCCCTGCT | |
| 5551 | AATGTGGAGA ATGGACTGAA GGCAAGGTGT TTTGTATATT TGTCTGTTTC | |
| 5601 | GTAGAGACAG GGTCTTGCTC TGTTGGCCAG ACTGAAGTGC AGTGGCACAA | |
| 5651 | TCACGGCAGC CTTGAACTCC TGGGCTCAGG CGAAACTCCC ACCTCAGCCT | |
| 5701 | CCTTACTCTC ACCATTGTGC CCTGCTAATT TTTTAAAAAA TTTATTTTGT | |
| 5751 | AGAGATGTGG TCTCACTATG TTGCCTAGGC AAGTCTTAAA TTCCTGGTCT | |
| 5801 | CAAATGATTC TCCTGCCTCG ATGTCCCAAA GTGCTGGGAT TACAGGTGTC | |
| 5851 | AGCTGCCATG CCCGACCTGT ATTTTTTTTT TTAATGGGGA AAAAGCCTTT | |
| 5901 | TAATAGTATG AGGTGTTTTC TGGTGTTTCT ACCATAAAGC TCTTCTGTAA | |
| 5951 | ATCAAAATGA GAATGTAATT ATTGATAGAG CAATGACCTT AGACTACAGT | |
| 6001 | GCAGACTTTT CATCTTACAT TTGGGCTCAT GAATTTTAGT ATAACTGATT | |
| 6051 | ATGACAGTGT TTTTTACATA GTTATGATCT AGAGCAGAAC TGAAAACAAA | |
| 6101 | ATAACACATA CTCTACATCA ATATATTCGT TCAGTAATAT CTGGGCTTGG | |
| 6151 | ATGAACCTGC AGAAGTAGGT AAAGCTGTCA GATATTTTCT TAAACCAACA | |
| 6201 | GAAAAGAAAT GTATATGACA GATGTTGTGT TTACTTACTT ATTTATTTAT | |
| 6251 | TTATTTATTT ATTTGAGATG GAGTCTCACT GTGTCACCAG GCTGGAGTAC | |
| 6301 | AGTGGTGTGA TCTCTGCTCA CTGCAACCTC CACCTCCCGG ATTCAAGCGA | |
| 6351 | TTCTCCTGCC TCAGCCTCCT GAGTAGCTGG GATTACAGGC GTGCACCACC | |
| 6401 | ACGCCTGGCT AATTTTTGTG TTTTTAGTAG AGACAGGGTT TCACCATGTT | |
| 6451 | GGTCAGGCTG GTCTCGAACT CCTGACCTCG GGATCTGCCC ACATCAGCCT | |
| 6501 | CCCAAAGTAC TGGGATTACA GGCATGAACC ACCACGCCCA GCCTGTATTT | |
| 6551 | ATTTTTTTAC CACTATGGAG TCCAATATGA AATTCTCACA ACTATGCATA | |
| 6601 | TACATTATTA ACATGTAAGC ACACCTAGGT ATAAATATGC ACATAGTCCA | |
| 6651 | TTAATTACAT CAGGGGAATT AAAAACATAC TTTCAAGTTA AAATGAATTT | |
| 6701 | TCAGGAAAAA AACTGCATTC ACAAATCTGA AATGTGAATA CAAAAATGAA | |
| 6751 | ATTGTGAAAT AAATAATGAA TATAGGTGTC ACCTAAACTT CCATAGTAAC | |
| 6801 | ATGCCTCCAA ATGTGGATTT AGTGATCATC CACCTTGGGA CAAGGGCTTT | |
| 6851 | TGAGAGCCTC CAGCTAAATT AGGGTTCCAG TAGCAGAGTG GCTGGCAAGC | |
| 6901 | CTGCCCTAAT GAATAATGCC AGCGAGCTGG GCGTGGGTAC TTACAGTGTG | |
| 6951 | CCCTTCATGG AATACTTTTT TTTTTTTTTT TGGAATGGAG TCTCGCCCTG | |
| 7001 | TTGCCCAGGC TGGAATGCAG TGGCACAATC TCAGCTCACT GCAACCTCGT | |
| 7051 | CCTCCTGGGT TCAAGCAATT CTCGTGCCTC AGCCTCCCAG GTAGCTGAGA | |
| 7101 | CTACAGCCCT GTGCCATCAT GTTCTGCTAA TTTTTGCATT TTTAGTAGAG | |
| 7151 | ACGAGGTTTC ACCAAGTTGG CCAAGACTGG TCTTGAATTC CTGACCTCAG | |
| 7201 | GTGATCTGCC CACCTTGACC TCCCAAAGTG CTGGGATTAC AGGCTTGAGC | |
| 7251 | CACTGCGCCC GGCCCATGAA ATACTTCTTA CCTGGCGGAC AGCCTAATAG | |
| 7301 | CCTAGCTGTC TAACCCATGG CTGGGGGTCC TTCACACTTG TTTATACTGG | |
| 7351 | CAGACGTCCC TGTGACTCTT GTCTGATCCA TGTCCAAGTT TATGCCTGTC | |
| 7401 | TGACCATTGC TCTGGCGCTG GGAGCCAGAC TGTGTTCCCA GCAACCCAGG | |
| 7451 | GAAAACCAGG CCTGGGCTGG GCCTGGGTTC CTGAGATGGA AGGTGCAAAT | |
| 7501 | TCAGTACACC ACCTCAATGC AAAACAAGTT CAAAGGCTTA TTACTTACAG | |
| 7551 | ATCCTGAGCA GGGAAGGTGC AATGAGTAGG GAGGGTCATC CTCCATCCTG | |
| 7601 | GGCTACATGA AGCGGGAATG AAGAGTCAGG CAAAAAGAAA GTGAGAGCTT | |
| 7651 | GTGGCAATGA GAAGTATATT ATGTAAGGGA CTAGGGTGTG GGTCAGGTTA | |
| 7701 | AGTTTGAGGG CAAATGCTTG AATGATCCCT TTAAAGGAAT GGGTGGGAAG | |
| 7751 | TGGGGAGCCC AGTTTGCCGG GAGGGAGAGA TGCCTCGAAG TTCTTATCTC | |
| 7801 | TGGCCACTGG CTTGGGCCAT CTGAGTGTGG CATCTACTTC TAATGCCTAG | |
| 7851 | GCAGCAACCT TTGCTGTGTC ATCTCCCTTA CACAAGGTTG GAAGCAGGGA | |
| 7901 | GACCGGTCAG GAAGCCTTTG GTGTAACCCA TGTTATTGTA ATATTCATTC | |
| 7951 | ATTTACTCAA CAGATGTTTA TTGTGCACCT ACTATGTGCT GAGGCCATGG | |
| 8001 | CAGGCAGGCT CTGGGGATGT GGCTGAGAAC AGGACAGAGC CCCTGGTCCT | |
| 8051 | TGATATCCTC AAGGATGCTC CCTCCTGGAG GCCATTAGGT TCCTGTTCCA | |
| 8101 | TGGTGTTCTG CTGGAACCCT CCGGTCCCAG AGTGTGCAGG AGCCTCCCCT | |
| 8151 | CCTGGCAAAG GGTCTTCTCT CATGGCACAA GGGCTGCAGT ACAGCCAGTC | |
| 8201 | AGTGGCTCCT GGTTCCTCAA ACTCAGTGAG CACTTGCCTG CCCTTCGTGC | |
| 8251 | TGCCCCTCAG CTTGGGATGG CCTGAGTCAA GACCAGCCAG GAGCTCCAGG | |
| 8301 | CTTCATGACC CCTTTCTTTC CCCCAGGGAG GCCCCGTCTG GCCCCTCCCC | |
| 8351 | AGAATGTGAC GCTGCTCTCC CAGAACTTCA GCGTGTACCT GACATGGCTC | |
| 8401 | CCAGGGCTTG GCAACCCCCA GGATGTGACC TATTTTGTGG CCTATCAGAG | |
| 8451 | GTAGAGGAGA CTCTCTCGGC TGGTGGATGG GAAGACTGAG GGGGTGGGTG | |
| 8501 | GGGGCTTGGA GGGGCTTCTC TGGGACAGCT GCACCCAGTG TGGGCAGCAC | |
| 8551 | TGGCTAGCTC TCTGGGCCCT ACGGGAGATG GCATGTGGCC GGCATTTGGA | |
| 8601 | GAGGGGCTTT TGATAAAGGT CTGGAGGTGG GGAAGATGTT GAATGAAGAG | |
| 8651 | CAGTGTACAG GTGACCAGTC TGCCGGGGCG GGGGTAAGTC TTTGAGGAAA | |
| 8701 | GTTGGTGTGG GGCATGGATG TAGCTGTGGG GGCCAGAGGA TGAAATTCTC | |
| 8751 | AAGTGGCTGG ATGAGGTGCT TGGAGCTGTC CCAGCTGATC AGTGAGGCAA | |
| 8801 | CTAGGTACAC GGCAGAGGAG CTGTTACCTG GGCAATTAGG CATCCCTCAA | |
| 8851 | TGATCACACT TTTTTTCTCT TTTTTTTTTT TTTTTGAGAC AGAGTCTTGG | |
| 8901 | TCTGTCACCC AAGCTGGAGT GCAGTGGCTT GATCTCGGCT CACTGCAACC | |
| 8951 | TCCACCTCCT GGGTTCAAGT GATTCTCCTG CCTCAGCCTC CAGAGTAGCT | |
| 9001 | GGGATTACAG GCATATGCCA CCACATCTGG CTAATTTTTG TATTTTTAAT | |
| 9051 | ACAGACGAGG TTTCTCCATG TTGCCCACGC TGGTCTCGAA CTCCTGAGCT | |
| 9101 | CAGGTGATCC ACCCACCTCA GCCTCCCAAA GTGTTGGGAT TACAGGCGTA | |
| 9151 | AGCCACCGCG CTTGGCCAAA TGGTCACACT TTTCCCGATG GGATCATTCT | |
| 9201 | CAATTTGGAA GCCCAGGCAG CCACAGCGAA TCCAGAGAAA TCTGACAATG | |
| 9251 | GAAGCAGATC CACCATCTTC GAACATAGAT GGGAATCGTT CAGAGTTCTT | |
| 9301 | TAGCAGGACA GTGAGATGAT AGAAGCAGAA GCTCGGGAGG ATTCACCTGG | |
| 9351 | AGTTGGTGAG GAGGGGAAAG CAGGAAGAGG AGGGGACCCA CCGTGTCCTC | |
| 9401 | AGGACCCGTC CTGTGCCAGG CCAAGTGCTA AGGGCCCTAC GTGAATATTT | |
| 9451 | CACTTCCTTC TCCCAATGTG ACCAGGCAGG CTCTGTGTTT TCCCCATTCT | |
| 9501 | AGAGGTGAGG GGGATTGAGC ACTGTGTCAA CACATGTAAT GAACTTAATC | |
| 9551 | TCACAGCAGC TCTCTGAGGA CAAGTTCAGT ACGCCTCTTT ACAGAGGAGG | |
| 9601 | AGACTGAAGC ACCAAGGGTG CATGTTGCTC AAAGTCACAC AGCTGGGCGT | |
| 9651 | AGTATGGCTG GAATAAATTT ATTAAGGAGT TGAAAGTCTA TCCTCTAGGA | |
| 9701 | CCAAGCATGG TGGCTTACAT CTGTAATCCC AGCACTTTGG GAGGCCGAGG | |
| 9751 | TGGGTGGGGA GATTGCTTGA GTCCAAGAGT TCCACACCAT CCTGGGTAAC | |
| 9801 | ATGGTGAAAC CCTGTCTCTA CAAAAAAAAA AAATACAAAA AATTAGTGAA | |
| 9851 | GTGTAGTAGC ATGTGCCTGT GTTCCCAGCT ACTTGGGAGG CTGAGGTGGG | |
| 9901 | AAGGATCACT TGAGCCCAGG AGATGGAGGT TGCAGTAACA AAGATCACAC | |
| 9951 | CACTGCACTC CAACATAACA ACAGAGCAAG ATCAAAAGGG TTTTTAGCTC | |
| 10001 | CCACTGAACG CCNCGTCATA NCCTTAGGTN NNNNNNNNNN NNNNNNNNNN | |
| 10051 | NNNNNNNNNN NNNNNMNNNN NNNNNNNNNN NNNNNNNNNN NNNNNNNNNN | |
| 10101 | NNNNNNNNNN NNNNNNNNNN NNNNNNNNNG AACAACAGAG CAAGATCCTA | |
| 10151 | AAAAGAAAGA AAGTCTATCC TCTGAACTTC TATGATATTT TTCATGTCTT | |
| 10201 | TTATACATTA GAATGGTGAT ATTCTAATTA TATAATTTTT TTCATTTGTT | |
| 10251 | AGTTGGAATT ATTTTATAAA GAGATGTATC CTCTCATCTG GTATTTGATA | |
| 10301 | TCCAGTCATA CTATTCAAAT AGGCAAGAGA GGATAAATGC TTAATTTTTT | |
| 10351 | TCCTTTATCA ATTTTCAAGA TAATGAATTG GTTCCTTATC ATCTCCCAAA | |
| 10401 | GGTGATTGCT AGTTTATTAT TATCATTATG AACTCAGGCA TTTAAACACA | |
| 10451 | TTTGGTGGTT TCAGTCTATT GCGACGTACT CTGCTCATTG AAGCTTGAAT | |
| 10501 | TGCCTCATCT CTGTCCAGTG GGAGTCTCAT CAAGTTTGCT CCTGAGTCCT | |
| 10551 | TTTAACTTGA CCCTAGTGGT CAAGTTAAAT CTTTCCAGAT TTAACAGATA | |
| 10601 | CCTTTCCAGC TGTCCATTAC GACAAGATGT TCCAGGTCCC TCTGGTACAA | |
| 10651 | TTCCTGACCT AAAACCTGCA GTCAGCCATT TCTCCATTTA GTAAGAAATG | |
| 10701 | GTTATAAAGA CTATAATCTG CATGCTAGCT ATGCTGATCA CTACTTAGCT | |
| 10751 | ATTGCTTTTG GTGTTTTCAG TGAACAGAGT GATGTGTGTA TACCACATAG | |
| 10801 | ACACACACAT GTACATACTT TTTTTTTTTA GACAGAGCTT CACTCTGTCA | |
| 10851 | CCCAGGCCAG AGTGCAGTGG CATGATCTCG GCTCACTGCA ACCTCCACCT | |
| 10901 | CCTGGGTTCA AGAGATTATC CTGCCTCAGC CTACTAAGTA GTTGGGATTA | |
| 10951 | CAGGCGCCCA CCACCATACC CGGCTAATTT TTGTATTTTT AGTAGAGACG | |
| 11001 | GGGTTTCACC ATGTTGGCCA GGCTGGTGTC GAACTCCTGA CCTCAAGTGA | |
| 11051 | TCTGCCCCCC TCGGCCTCCC AAAATGCTGG GATTACAGGC ATGAGCCATC | |
| 11101 | GCACCCAGCC TACATGTACA TAATTTTTAA GATAAAATGC CTAATGAGTT | |
| 11151 | ATACGGGTGC TTCCCATCTA AATTTAGTTC CTTAGGATTT TTACCTGACT | |
| 11201 | TCTATGGTAC ATCTATATTT TCTTTCTTTC ACACTGAGAA TCCTGTTTCT | |
| 11251 | CAAGGACAGG GGACATGATA GAACTAGAAT GACCCATAAT TACTCATTTT | |
| 11301 | CTTTATCCCA AAACATACAT ACTTGCCTCT TAATAGTTTC TTGCTCTTTT | |
| 11351 | CGCCCAAAGG GTTTGTGATG GTCAATATTA GGTGTCAACT TAATTGGGTT | |
| 11401 | GAAGGATGCC TAGATGGCTG TTAAAGTTTT GTTTCTGGGG GTGTCTGTGA | |
| 11451 | GGGTGTTGCC AGAGGAGACT GACATTTGAG TCAGTGGACT GGGAATGGAA | |
| 11501 | GACTCGTCCT CACTCAGTGT GGGTGGGCAC AACCCAACTG GCTGCCAGGC | |
| 11551 | TGGCTGGAAA GCAGGTGGCA GATGGTGGGA TAGCTTCACT TGCTGGGTCT | |
| 11601 | TCCAGCTTCC TTCTTTCTCC CGTGCGGGAT GCTTCCTTCT GCTCCTCCTG | |
| 11651 | CCCTTGAACA TCACACTCCG GGTTTTTTGG CCTTTAGACT CTTGGACTTA | |
| 11701 | AGTTAGTGGT TTGCTGGGGG CTCTCGGATC TTTGGTCACA GACTGAAGGC | |
| 11751 | TGCACTTTCA GCTTCCCTGG TTTTGAGGGT TTCAGATTCG GACTGAGTCA | |
| 11801 | CTATGGCTTC TTTCTTTCCC ACCTTGCTGA CGGCCTATCG TGGGACTTCG | |
| 11851 | CCTTGTGATC GTGTGAGCCA ATTCTCCTTA ATAAACTCCC TTTCATATAT | |
| 11901 | ACGTATAACC TATTAGTTCT GTTCCTCTGG AGAACCCTGA CTAATAAAGG | |
| 11951 | GTTGTTGCTT TTTCTTTAAA ATCTAGTAAT TTTATTTGAC TGTGTGTTGG | |
| 12001 | TATTGCTCAT TCATTCTGAG TTGATATTTT TAGGCACTCA ATATTCTCAC | |
| 12051 | TTAATACATG GTTCCAAGGC ATTTTTATTT TAGGAAGGTT TTCTTAAATT | |
| 12101 | ATAGTTTTAG TATTTGTTCT ATTCTCTTGT TTTGATTTTC TTCTTTAGGG | |
| 12151 | ACTCATATCA CTTGTATGTT GGATCTTCTT TTTCTGTGTT CAGTATTTGT | |
| 12201 | CTTTTGGGCA CAGAGACTCA CACCTATAAT TCCAAGACTT TGTGAGGCAT | |
| 12251 | AGGTAGGAGG ATCGCTTGAG CCCAGGAGTT TGAGACCAGC CTGGGCAACA | |
| 12301 | TGGTGAGGCC CTGTCTCAAA TTAAAGAAAA AGGAGAGAAT ACTTGTCTTT | |
| 12351 | TTCTTTCAAA TGCCTTTTAT CTGTCTGTCT ATCTACTATT CTGCTCTCTA | |
| 12401 | AATGAAATAG GTTTCACTCT TGAGTTTTTA AAAAACTGTG TGCTTCCATG | |
| 12451 | TGTGAGATTA TTCAACATCT TATTTGTAAT CTTTCTCTTG GTTACATTTA | |
| 12501 | TTTTTCCTGA AAACTCTAGT CTGCTTTTAG CTGACATGTT TGTAGCTAAG | |
| 12551 | AGCGCACATT TCTTATCATA GCTTGCCGTG CTGAATTAAT TCCAATTTTC | |
| 12601 | TTTTAAAACC AACATTATTG AGTTAAAATG TATATAGAAT AAACTGTTCC | |
| 12651 | CATTTTAAAG TATACAATTT GATGAGTTTT GACAAAAGTG GGCACCCACG | |
| 12701 | TACCCACCAC CACAATCAAG ATGTAAGACG TTCTCTATCA CCCCAGAAAG | |
| 12751 | TTCCCTCATC CACTTTGCAT TCAGGCCTCC AGATCTAGGC AACCACAGAT | |
| 12801 | CTGCTTTCTG ACACTGTGGA TTAAACTTTG CCTGTTCCAG AATTTCATAT | |
| 12851 | AAATGGATGT GTATAGTATG TACCCTTTCG TGTCTGGCTC CTTTCCCTCA | |
| 12901 | GCATAATGTT TCTGAAATTC ACCCACATTG TTACATGTAT CAGTAGTTAA | |
| 12951 | TTCCTTTTTA TTGCTGAGTA GTAATGCCAT TGTATGACTA TGTATGACAT | |
| 13001 | TTGTTAATCC ATTTTCCCGT CAGTGGATAT TTGGGTTGCT TCCAGTTCTG | |
| 13051 | GGCAGGTATT CATTTGCTAG GGCTGCCATA TGCTTGCCCT CTGGCCTCCC | |
| 13101 | AAAATTTGTG TCCTTTTCAT ATGCAAAATA CATTCACCCC CTCCCAACAG | |
| 13151 | CCCCAAAACT CTCTTTTTTT TTTTTTTTTG AAACAGAGTT TTGCTCTTGT | |
| 13201 | TGCCCAAGCT GGAGTGCAAT GGTGTGATCT CGGCTCACTG CAACCTCTGC | |
| 13251 | CTCCCGGGTT CAAGAGATTC TCCTGCCTCA GCCTCCTGAG TAGCTGGGAT | |
| 13301 | TACAGGCATG CGCCACCACG CCTGGCTAAT TTTTTATATT TTTAGTAGAA | |
| 13351 | ATGGGGTTTC ACCGTGTTAG CCAGGCTGGT CTTGAACTCC TGACCTCAGG | |
| 13401 | TGATCCGCCT GCCTTGGCCT CCCAAAGGGC TGGGATTACA GGCATGAGCT | |
| 13451 | ACTGCACCTG GCTAGCCCCA AAACTCTTAA CCCATTTCAG CATCTACTCT | |
| 13501 | AAGTCCAAAG TCTCATCTAA ATCAGGTATG GGTGTGACTG GAGGTGTTAC | |
| 13551 | TCATCCTGAG GCCAAATTCC TCTCCACTTA TGAACCTGTG AAACCAGACA | |
| 13601 | GGTTATGTGC TTTGAAAATA AAGTGATGGG ACATGCATGG GATAGACTTT | |
| 13651 | CCCATTCCAA AAGAGAAAAA TAGGAAAGAA GGAAAGAGTG ACAGGTCCCA | |
| 13701 | AGCAAGTCTA AAACCTCGCA GGGCAAATTC CATTAGATTT TAAGTTTCAA | |
| 13751 | GAATAGCCCT CTTTGGCTCA GTGCTCTGCC CTTTGGGCCC ACTGGGGCGG | |
| 13801 | CAGCCCTATC CCCTTTGCCC TGGGTGGTGA CCCTACCCTC GAGTCACTGG | |
| 13851 | TTAGCAGCAG CCTAGCCTGC TGAAACTAAG GAGGGGACAG TGTTGCCTCC | |
| 13901 | AGGTCTTTGG TGGCAGTGAC AACCCTGCTG ATCTCTGAAT CATCTTCCAG | |
| 13951 | GAAATTTTTC CCTATACTTG AAGGATATTG CGTGTTCACA GCCAAATAGC | |
| 14001 | TCCAGCTCTT GTCCCTTTCT TTAGAATCCC AGAAGTCCAA CAGCCTTCCT | |
| 14051 | TCATTCTGTC CCATCTCTGT CCCCTTTAGT CAAAGCTGGA AGTGCCTCTG | |
| 14101 | CTGGTATAAT CCCATCAGTA TGTCTAATTT CTGCTTAAAT GGCTGATTAA | |
| 14151 | GTCTATGAGT TGCACCTCTG ATCTCTTTAT CAAAAGGTTG TTCTAGCCAC | |
| 14201 | AACCTTAGTG TCCTCCCCAG AACATGCTTT CTCATTTTTT TTTTTGCAAT | |
| 14251 | GTGGATAGGC TGAAAATTTT CCAAAGCTTC AAGTTCTAGT TCCTTTTGGC | |
| 14301 | TTACCAATTC TTTTCATATA TCTCTTCTCT CACATTTTAC TATAAGCAGT | |
| 14351 | AAGAAGAAAC CAGGTTGTAC CTTCAGCACT TTGCTTAGAA ATCTCTTCTG | |
| 14401 | CTAAGCATCC AAGTTTATGT CTTTTAAATT ATCTTTTTGT TATTTATTTT | |
| 14451 | ATATTATCAT TTTTGAGATG GCTAGCCAAT GATCTTTTAA CTTCTAATTT | |
| 14501 | CTGCAAAACA CTAGAAGACA ATTCAACCAG TTCTTTGCCA CTTTATAACA | |
| 14551 | AGGATCACCT TTCCTCCAGT TTCCAATAAC ACATTCCTCT TTTCCACCTG | |
| 14601 | AGACCTCACC AGAATCACCT TTAATGTCTA TATTCCTACC AATAGTCTTT | |
| 14651 | TTAAGGCAAT ATAGGCTTTC TCTAACATGC ACTTCAAACT TCAAGATTCT | |
| 14701 | ACCCATTATG CAATTCCAAA GCCACTTCCA CATTTTTAGG TATTGATTAC | |
| 14751 | CTCAGCACCT CATTTCTGGT GCCCAAATCT GCACTGGTTT GCTAGGGCTG | |
| 14801 | CCATAACAAA GTACGACAGT CTGGGTAAAC AACAGAATTT TATTTTCTCA | |
| 14851 | AAATTCTGGA GGTTGGAAGT CCAAGGTCAA GGCGTTGCTA GGTTTAGTTT | |
| 14901 | CTCCTGAAGC CTCTCTCCTT GGCTAGCAGA TGGCTGCCTT CTTGCTGTGT | |
| 14951 | CCTCACGTGG CTTTTTCTCT GTGTGTGTTC ACTCTGGTAT CTCTTCCTCT | |
| 15001 | TCTTACAAGT ACACCAGTCC TACTGGATTA GGGCCCCAGC CTTATTACTT | |
| 15051 | CATTTAACCA TAATTACCTC TTTAAAGCTC TTATCTCAAA ACACAATACC | |
| 15101 | ACTGGGGATG AGGTCTTCAA CATATGAATT TTGGGGGAAC TCAATTCGTC | |
| 15151 | CATAATAGGG CTATTATGAA TTAAGCTGCT GTGAACATTC ATGTACAAGT | |
| 15201 | CTTTGTGTGG ATATGTTTTC ATTTCTCTTA GATAAAGATC TAGGAGTATC | |
| 15251 | AGCCTGGGCA ACATAGTGAG ACCCCATCTT TACAAAAAAT TTTCAAAATT | |
| 15301 | AGCCAGGCAT GGTGGCGTAC ACCTGTAGCC CTGCCATCTC AGGAGGCTGA | |
| 15351 | GGTGGGAGGA TCCCTTGAGC CCAGGGGTTT TAGACTGCAG TGAACTATGA | |
| 15401 | TTGCACCACT GCACCCCAGC CTGGGTGACA GAGTGAGACT CTGTCTCTAA | |
| 15451 | AAAAAAGAGA GAGAGGGGAG GAAGGAAAGA AGAAAGAGAG GGAGGGAAGG | |
| 15501 | AGGGAGGGAG GGAGGGAGAA GAAAAATGGA TCTAGGGTTA AGATTTAGGA | |
| 15551 | GATTAGGTAA TGAATGTGTA CTATTACAGG GAACTGTCGA GCTGTTTCCA | |
| 15601 | AAGTGACTGT ACCATTGTTC ATTGCCACCA ACAATACATG AGAGTTCTAG | |
| 15651 | TTACTCCATG TGCTTGTTAC ACTTAGTATT ATCAGTCTTT TTCATTTTAA | |
| 15701 | CCATTCTAGT GAGTATGTAG TAGTATTTTA TTATGGCTTT AATTTACAAC | |
| 15751 | TCCCTAATGA TGAATGATGT TGAACATCTT TTCATGTGCT TATTGGCCAT | |
| 15801 | TCATATATCT TTTGTGAAGT GACTATTCAA ATATTTTTCC ACTTTTTATT | |
| 15851 | AGGTCATTTA TTTTCTTATT ATTGAGTTAT CTATGAATAC AAATCCTTTA | |
| 15901 | TCAGTGTATG TATTGTGATT TTTTTCCCCA GTGGCTGGCC TTTTCATTTT | |
| 15951 | CGTTAGGCTT TTTTGGTGGG TTTTTTTTTT TTTTTTTGGA AGAGAAAAAT | |
| 16001 | ATTTTAATTT GATAAAATCC AGTATATCAG GTGTTATAGA CTGAATTATA | |
| 16051 | CTCTACCCCA CAAATTCATA TGTTGAAGCC CTAACCTCTA AGTGACTATT | |
| 16101 | TGGAGATGAG CCTTTAAGGA GGTAATTAAA GTAAAATGAG ATCATAAGGG | |
| 16151 | TGGGCCCTAA TCTAATAGGA CTGGTGTCTT TATAAGAAGA GGAAGACACC | |
| 16201 | AAGAGCGCAT GCACACAGAA GAACGGCCTT GTGAGGACAC AGCAAGATGA | |
| 16251 | CGGCCATCTG CAAGCCAAGG AGAGAGGCCT CAGTAGAAAC CAAACCTGCT | |
| 16301 | GATGCCTTGA TCTTGGACTT CCAGCCTCCA GATTTCTGTT GCTGAAGCCA | |
| 16351 | CCCTGCCTGT GGTGTCTTAC CATGGCAGCC CTCACAGACT AATATATCAG | |
| 16401 | ATTTTTTTCC TTCAACAGTT AACGCTTTTG GTGTCCTAAG CAATATTCGC | |
| 16451 | CTGACCCAGG GTCATGAAGA TTTTTCTTCT ATGCTTTCTT CTGGAAGTTC | |
| 16501 | TATAATTTTA GCTTTTACAT ATTTTTTTAA CTTTCCTTCT TCTTGCCTTC | |
| 16551 | TGTTTCTTTT AAGGCATCAT CTATTGTGTT AATTTGTTCT TGTATTCCTT | |
| 16601 | CTGATTTATT CTTCACTTCT GAAATGAATT TTGCTTTTTA AAAATATATA | |
| 16651 | TAATTCTTTT CTGTGTCTGA GTTTTTCTAA TTAGGTTTTA TGTGGTTTTT | |
| 16701 | TCTTGTCCTG CATCACTTTT TACTGTCTTT TGCCCATTTT GAAGTATCAG | |
| 16751 | GTTCCAGTTT TGATCTGTTC ATGGATATGT TTTTGTGACA TGTTTCTTCT | |
| 16801 | GGCTTCTTAT CATTTATTGC TTAGCTTATT AATTTCTATT CTTTCTTATT | |
| 16851 | TTCTATTATA AGTATTTAAA GCTATATGTT TTCCTCTAAG TATTACTTAG | |
| 16901 | CTGTCTTATA CGTTTTCATT TGTGTTATTT GGTGATCATT CACTTTCAGC | |
| 16951 | TATTTATTAA TTTCCATTAT AATTCTTTCA TCTATGGGTT GTTTTAAAAA | |
| 17001 | ATATTTTTAA GGCCAGGTGT GGTGACTCAC ATCTGTAATC ACAGCACTTA | |
| 17051 | GGGAGGCTGA GGTGGGAGGA TTGCTTGAGG CCAGAAGTTT GAGACCGGCC | |
| 17101 | TAGGCAACAA AGTGAGACCC CCTCTCTACA GAATATTTTT TTAAAATTAG | |
| 17151 | CTGGGCCAGG CGTGGTGGCT CATCCCAGCA CCTGTAATAC CAGCACTTTG | |
| 17201 | GGAGGCCAAG GCAGATGGAT CACCTGAGGT CAGGAGTTCG AGACCAGCCT | |
| 17251 | GGGCAACATG GTAAAACCCC ATCTCTACTA AAATATAAAA ATTAGCCAGG | |
| 17301 | TGTGGTGATA GGTGCCTGTA ATCCCAGCTA CTTGGGAGGC TGAGGCAGGA | |
| 17351 | GAATTCTTTG AACCCAGGAG GAGGAGTTTG CAGTGAGCCG AGATTGCACC | |
| 17401 | ACTGCACTCC AGCCTGGATG ACAGAGCGAG ACTCTGTCTC AAAAAAAAAA | |
| 17451 | AGAAAAGAAA ATTAGCTGGG TGTAGTGGCA GGTACCTGTG GTCCCAGTGA | |
| 17501 | CTCAGAGACT GAGGCAGGAG GATCACCTGA GCCCAGGAGT AGAGGCTGCA | |
| 17551 | GTGAGCTATG TTTGTGCCAC TGCACTCCAG CCTGTGCAAC AGAGCAAGAC | |
| 17601 | GCTGTCTCAA AAAATATATA TTTTTTTAAA TTTTCAAACT TCCTTTAGTT | |
| 17651 | CTCTTTTTGT TATTAACTTT TAACTGAATG TTTTGCAATC AGAAGAAATA | |
| 17701 | CTTTATGAGA TACCTATTCT TTAAAATTTC TTAAGAATTG CTTTGTGTTA | |
| 17751 | ATATTTTGTT AATAGTTCAC ATGTGGTTCA ACCAATTTGT TTAGTTAGTT | |
| 17801 | CTGTATATGT TCATTAGACC AACTTGATAA CTGTGTTGTT CTTTATTTAT | |
| 17851 | TTATGTATTT ATTTTTCTTT GTCTATTCAT CAATTGCTGG GTGAGATGTA | |
| 17901 | TTAAAATTTC TTGTTGTAAG TGTGGCTGTT CACTTTCTAC CTGTAGTTTG | |
| 17951 | TCTGTTTGCT TTATAGAGGG TGAAGTTGTT TAGTAGGCAC ACATAAGTTA | |
| 18001 | GAATTTTTCT GTCTTCCTGG TGAATGGAAT CATTTATCAT TATCTAATGT | |
| 18051 | TCTTTTCATC TTTAGTATTG CTTTGGACTT GGAAGTCTGT ATTTTGTCTC | |
| 18101 | CTGTTAATAT AACTACACTG GTTCCTTTGG TGTGAATATT TGCATAGTAT | |
| 18151 | AACATTTTCC ATGAAGAAAC AAAACAGAGG AATTGGTTCT TTCTCAAAAT | |
| 18201 | CTGATCTTTG TGTCAGCCCC CATCTCAGCC TTCTCCATTC ATCCTTGGTC | |
| 18251 | ACTCCCCAAA CCCAGGAGCA ATCCTTGATT CTCCTTTTCC CCACATTCTA | |
| 18301 | CATCCAATCC GTTAGCAAGT TCTATTAGTT CTATTATTAC CTCCAAAATA | |
| 18351 | GATATTGAAT CCAGCCCTTT CTCACTGTCT CCACCATCAT CCTGTCTCAC | |
| 18401 | ATCCCTACCA TGGCCTCCTT GCTGGTTGAC CAGAGTGATC TTGTAAAAAC | |
| 18451 | ATGTTAGGCC AGGCACGGTG GCTCCTGCCT GTAATCCCAA CACTTTGGGA | |
| 18501 | GGCCAAGCGG GTGGGTCACC TGAGGTCAGG AGTTGGAGAC CAGCCTGGCC | |
| 18551 | GACATGGTGA AACCCTGTCT CTACTAAAAA TACAAAATTA GCCAGGTGTG | |
| 18601 | GTTATGCTGG CCTGTAATCC CATCTACTCG GGAGGCTGAG GCAGGAGAAT | |
| 18651 | CACTTGAACC CAGGAGGCGG AGGTTGCAGT GAGCCAAGAT CATGCCACTG | |
| 18701 | CACCCCAGCC TGGGCAACAG AACAAGACTC CATCTCAAAA AATAAAAATT | |
| 18751 | AAAATAAAAT GTTAGGCTCC CTGGGTCTCT GGCTTAGTCC ATTTGTACTG | |
| 18801 | CTTTAACAAA ATACCTTAGA ATGGTGTAAT TCTAATAATT GCTATTAATA | |
| 18851 | AATAATAGCA ATTAATAAAT AATAGCAATT TCCTTCTCAC AGTTCTAGAG | |
| 18901 | GCTGGGAAGT TCAGGGTCAA GGTGGCACCT GACTCCGTTC TGGTAAGGGC | |
| 18951 | GGCTCTCTGC TTCCAAGATG GTGCCTTCTC GCTGCGTCTT CGCATAGCGG | |
| 19001 | AAGGGCAAAC ACTGTGTCCT CACGTGGCAG AAGAGATAGA AGGGCCAGGC | |
| 19051 | AGCTCTCTGA AGTATCCAGG TTGGAGTCAT GGACCTGCAT GTTCCCCTCT | |
| 19101 | GACATCCACA GAGTACCTAT CATGGTCCTT GGCATGCAGC AGGTGGCCCA | |
| 19151 | TAAACGCCTG AATGAACAAA CATATAGTAA TGGTCGCTAG TACTAGGAAT | |
| 19201 | AGCAGCCACC GCAACAGTCC TGTGAGGGAG GCATTACAGA TGAGGAAACT | |
| 19251 | GAGGTTTAGG GGCAAGGACC TGCCCATGGT CCCAAAGCTA GGGAGGGACA | |
| 19301 | GGGCTGGGAT TCCCACTCCC ATCCATCTGG CTCCAGAACC TGAGCTCCTG | |
| 19351 | ACCAGGCTGT TCTTATCCTG TCTCAGCCAG TGGCTGCCTG TCTGGACGGA | |
| 19401 | TGGACCTAAA GTCAGTCCAG CCAAACAGAG GGAAGCATGA TCAACTGTTC | |
| 19451 | TCTAAGTTCC CTGACCCGGA GAGGCTGAGT CCATGGCCCA AGCTCTCCTC | |
| 19501 | TCTCCTCCCC CAGCTCTCCC ACCCGTAGAC GGTGGCGCGA AGTGGAAGAG | |
| 19551 | TGTGCGGGAA CCAAGGAGCT GCTATGTTCT ATGATGTGCC TGAAGAAACA | |
| 19601 | GGACCTGTAC AACAAGTTCA AGGGACGCGT GCGGACGGTT TCTCCCAGCT | |
| 19651 | CCAAGTCCCC CTGGGTGGAG TCCGAATACC TGGATTACCT TTTTGAAGGT | |
| 19701 | AGGTCTGTGG GTAAGGGACT GAGTGGAAGG CTGTCCATCC CATCGGGGAG | |
| 19751 | CTGTGCTCAG TGCTCAGTGG TTCTGTTCTC CTGACCATCT GTCTCCCACT | |
| 19801 | TCCCCAAAGC AGAGGGCAGC TCCCTGGGCC AGGCCCTTTG AGATGGGGTG | |
| 19851 | TGGGACCAGC AACAGCGAGG GACCATGTCT GGTAGCCTGT CAGGGAGTTA | |
| 19901 | GGGGAGCTCC AGCCAGCACC AGCAATCTCA CGTGCACCCT CTGCTAACAA | |
| 19951 | TGTTCATTAT TTTCAGTTGA GCACCATTTT GGTCATGGAC TACACAAGGC | |
| 20001 | ACTTTATATG CTTATTCCTA TTTTTTTATG TTCAGCTTCT CTCCTTAAAA | |
| 20051 | ACAATGTTTA AAACCAATTC TGGGCCAGGC GTGGTGGCTC ACGCCTGTAA | |
| 20101 | TCCCAGCACT TTGGGAGGCC AAGGCAGGTG GATCACCTGA GGTCAGGAGT | |
| 20151 | TTGAGACCAC CCTGGCCAAC ATGGCAAAAC CCCGTCTTTA CTAAAAATAC | |
| 20201 | AAAAATTAGC CAGGCTTGGT GGCAGGCACC TGTAATCCCA GCTACTCGGG | |
| 20251 | AGGCTGAGGC AGGAGAATCG CTTGAACCCA GGAGGCGGAG GTTGCAGTGA | |
| 20301 | GCCAAGATCA CGCCCCTGCA CTCCAGCCTG GGCGACAGAG CGTCTCAAAA | |
| 20351 | GAAAAAAATT AATAAACAAA GAAAAAAAAA CAAATTCTGT TTGCAAAAGT | |
| 20401 | ATTTTCTATA CACTGTAGAA ATTTGTGGGG TGTGGGGGGG TAAAGATGAT | |
| 20451 | AGAAAAAAAA ATGTCCCATG CTTACTGGCA GAAATCATGT ATTGACATTG | |
| 20501 | GGTGAGGAGG GCACTTTTTT TTTTTCAGTC TATTTTTAAT CTTCACAGCA | |
| 20551 | AACTTGTGAG GTTCATTTCC ATCAACCTGA GACTCACAGA AGCTAAGAAA | |
| 20601 | CTTGATACCG CTAGTAACCA GTGGACTTGA TACCGCTAGT AACCGGTGGA | |
| 20651 | CATAGATGTG AACTGGATCT TTCTGACCTC GGGCAGGGCC GGGTAACAAG | |
| 20701 | GGGAGGATAA ATGCCCAGAC AGTGTCCTCA GAGAGCTGAG AGCTGTAACT | |
| 20751 | TGCTGCCCGG GCTTCTCACA GTGTTCAAGG ACAAAATAAG GCTTTAAGAG | |
| 20801 | AGAAGAGGGA CAGACTGATT GCAGGGCAGC AGGAAGAGAT GGTAGAGAAG | |
| 20851 | GAAGAAGAGA TGATTCGTGT GGAAAGAAGC TGGCTCGGTG GATGGATAAA | |
| 20901 | AGAAGGGAAG GACAGATGGG TAAGAAGAAA GGGAGGATGG AGGGGATGGA | |
| 20951 | GGAGGAAGCA ATGGAAAAAT GGGAAGGAAG GAGGTTGGAT GGAAGGATAG | |
| 21001 | ATGCCTATTA GGAAGGAAAT ATGTGTGGAT AGAGAGATGG AGGATAGGAA | |
| 21051 | GTATGTTAGT CAAGGTTCTC CAGAGAAACT GAACCAATAG GATATATACA | |
| 21101 | GATACACTAA GAGGAGGCCA GCCGGGCGCG GTGGCTCAAG CTTGTAATCC | |
| 21151 | CAGCACTTTA GGAGGCCGAG GCGGGCGGAT CACGAGGTCA GGAGATCAAG | |
| 21201 | ACCATCCTGG CTAACACAGT GAAACCCCGA CTCTACTAAA AATACAAAAA | |
| 21251 | AAAATTAGTT GGGCGTGATG ATGTGCGCCT GTAGTCCCAG CTGCTGGGGA | |
| 21301 | GGCTAAGGCA GGAGGATGGC GTGAACCCAG GAGGCAGAGC TTGCAGTGAG | |
| 21351 | CTGAGATCGT GCCACTGCAC TTCAGCCTGG GTGACAGAGC AAGACTCCGT | |
| 21401 | CTCAAAATAA ATAAATAAAT AAATAAAAAG AGGCCAGCCA TGGTGGCTCA | |
| 21451 | CACCTGTAAT CTGAGCACTT TGGGAGGCCG AGGCGGATGG ATCATTTGAG | |
| 21501 | ATCAGGAGTT CAAGACCAGC CTGGCCAACA TGGTGAAACC CTGTCTCTAC | |
| 21551 | TAAAAATACA AAAGTTACCC GTGTGTGGTG GCACACACCT GTAGTCCCAG | |
| 21601 | CTACTCAGGA GGCTGAGGCA GGAGAATTGC TTGAACTTGG GAAGCAGAGG | |
| 21651 | TTGCAGTGAG CTGAGATCAC GACACTGCAC TCCAGCCTGG GTGACAGAGC | |
| 21701 | AAGACTTTGT CTCAAAAAAA AAAAATTTAT AATAAGAGGA GATTTATTAT | |
| 21751 | GGGAATTGGC TCATGCAATC ACAGACACAA AAATGTCCCC CAGCATGCAG | |
| 21801 | TCATGGGCTG GACAACCAGG AAAGCTTGTG GTGTGATTCT GTCTGAGTCT | |
| 21851 | GAAGGCCCAA GGCCAGGGGA GCAGTGGTGT AACCCCCAGT CCGAGGCCAC | |
| 21901 | AGGCCCGACA ATCAGAGGGG CCACTGATAT AAGTCCCAGA GTCCAAATGC | |
| 21951 | CGGAGAACAG GAAGCTCCAA CGTCCAAGGA CAGGAGAAGT TGATGTGCCA | |
| 22001 | GCTCAGGAAG AGAGAATGTG AATGTGCCAT TCCTCCTCCA TTTTTTGTTC | |
| 22051 | TCTTTGGGCC GTCAGTGGAT TGGATGATGC CTGCCCACAC TGGTGAGGAC | |
| 22101 | AGATCATCAC CAAATCTGCC GATTAAAATG TTAATCTCTT CTGGAAAAAT | |
| 22151 | CCTCACAGAT GGGCCCAGAA ATAATGTTTT ACTGTCTACC TGGGTATCCC | |
| 22201 | TTAGTGCAGC TAAATTGACA CATAAACTTA ACCATCACAG GCCAGGCACT | |
| 22251 | GTGGCTCACA CCTGTAATCC CATCACTTTG GGAGGCCAAG GTGGGAAGAT | |
| 22301 | CCTTTGAGGA TGAGGTAGGC AGATCACTTG AGCCTAGGAG TTCAAGACCA | |
| 22351 | GCCTAGGCAA CATAGGGAGA CCTCGTCTCT ACAAAAAAAA AAAAAATTTA | |
| 22401 | AATTCGCTGG GTACGGTGGT GGGCACCTGT GGTCCCAGCT ATCTGGGAGG | |
| 22451 | CCAAGGTAGG AGGATGACTT GAGCCCAGGA GGTCAAGGCT GCAGTGAGCC | |
| 22501 | ATGATTGTTC CATTGAATTC CAGCCTCGGT GACAGAGCAA CACCCTGTCT | |
| 22551 | TAAAGAAAGA AAAAATTTAA CCATCACAGA AGGCAGAAGA AAAGGCAGAT | |
| 22601 | GGGTGGATGA GATGGGTGGG TAGATAGTAT AGAAGAAAAG CGGGACATCC | |
| 22651 | AGGCAGGGAA GGAAGGGCTG GAGCGAAGGA GAAGCAAGGA AGGAAGGAAG | |
| 22701 | GAGAGACAAG AAGGAAGGAT GTGTAGAAAG GTGGAAGAGA AAAGAAGAAT | |
| 22751 | GGATGTATGG GAAGAATGGA TGAGTAGGTT AGAAGGCTCA CTGGCTAGAT | |
| 22801 | AAAAGGTGAG AAGTATAAAT GAATAATAAG AAAGGAGGCA TAGGAAGAAA | |
| 22851 | AAAATATTGG TTAGAAAGGA TGATTGAGAA GAAAGGGTGG TTGGGAAGGA | |
| 22901 | AGGAAGGAAG GATGGATGGA TGGATGGATG GATGGGAAGG AAAGGAAGGA | |
| 22951 | TAAGAAGGCA GACAGGAAGG CTCTCTGGCT AGAAGAATGG CAGACAAACC | |
| 23001 | ACAATAATTG CTGAATGGGT AGGAATAAGA CATTAGAAGA ATAAAGGGAA | |
| 23051 | AGACACAAAG ATATTTAAAA TGTTTTCATT AATTTTTTGC CTCCTCCCTG | |
| 23101 | AATTTCTCCT GATTCTTCAG CCCCACATCC CAAGCCAGGG TGATCCTTCC | |
| 23151 | TGCCTTTACA CTCCCTCCAC ACTTTTTCTG CTCTCATATG TGGCCGTGGT | |
| 23201 | CACTTTCTTT TGGTAGTTTG CATATTTCAT TTACCCCAAA CTTTCAGCTC | |
| 23251 | CTGAAGGTCA GGATACAAGG AGGCCTCATC TCCGCATTCC CCTCAGCTCC | |
| 23301 | CTTCCTGAAG CTTGATACCT AGTCAGTACC CAGTGGATGT TTCCTAAACA | |
| 23351 | TGTAAGTAAT GACATCATGA AGAAGCCACA TGTTTACCTT GACCACAAAC | |
| 23401 | ACAGGGCAAA GGTGACTAGT GTGGTCAGAG ATCCCTGCTG GCTGGGAATC | |
| 23451 | AGGGAAGGCT GCATGGAAGA AGTGGCATTT TAGTTAGAAC TTGAAAGGTG | |
| 23501 | GTGTATTTAG TTTTCTCTGG CTGCCATATT CCTTGTCACA TTGCCCTCTC | |
| 23551 | CATCTTCAAG CCACTGGGCA AGGCTAGAAG GCCCTCAACA GACTATCGGT | |
| 23601 | AGGAATGTGG AAGTTGAAGA CTCAGAGTGC AGAAAGAAAC AAGTAGCATT | |
| 23651 | TTAGAGAAAA GCTAAATCCC CTCCAAGAAT ACCTCAATCA TCGTGAAGAG | |
| 23701 | CCTGTTAGTA GACGCACTAA CACTCAAGGC ACTGCTTCAC AAGGTAAGGA | |
| 23751 | ACGTGTAATT GAAAACTTGA GAAAGGAAGA AACTTGTTCT GTACTGGCAG | |
| 23801 | AAAGCTTAGC AGAATTGTGT CCTGCAGTCA TATGGGACAC AGAGCTTGTA | |
| 23851 | AATGATGAAT TTGAATGCTT ATCCGAGAAG GTTTCCAAAT AAAATGTGGA | |
| 23901 | AGGCACGGCC TGGTTTCTTC CTGCCTCTTA TAGTAAAATG CAAGAGGAGA | |
| 23951 | GAGAGAAAAT GAGGGAAGAA CTTAAACAGA AAGGAACCAG GACTTGATGA | |
| 24001 | TTTGGGAGGT TCTCAACCTA TGCAAAAAAC AATAAAATTA AGAGATTGTA | |
| 24051 | GCTGGGCACA GTGGCTCATG CCTGTAATCC CAGCACTTTG AGAGTCCGAG | |
| 24101 | GCGAGCAGAT CACCTGAGGT CAGGAGTTTG AGACCAGCCT GGCCAATGTG | |
| 24151 | GGGAAACTCC GTCTCTACTA AAAATACAAA AATTAGCTGG GTGTGGTGGC | |
| 24201 | GGGCACCTGT AATCCCAGCT ACTCAGGAGG CTGAGGTGGG AGGATCACTT | |
| 24251 | GAACCCAAGA GGCGGAGGTT GCAGTGAGCC AAGATCATGC CACTGCACTC | |
| 24301 | CAGCCTGGGT GGGTGACAGA GCAAGACTCC ATCTCAAAAA AAAAAAAAAA | |
| 24351 | AAGAGATTGC TCCCAAAAGT GTGACATAGA GAAACAGCCA AGTATGTGAT | |
| 24401 | TATACCAAAC TTCAGGAAGA TAAAAGATCA AAGTACTCAG TCGCTCAAAA | |
| 24451 | GGCTCTTTGA AGAGATTAAG ATTATAACTC ACAGTCCCCT TCAATCAAAC | |
| 24501 | CAGGGGACTT CTAGGAAGCT GAACAGCATT GTCCCTCAGC CATATCAGCT | |
| 24551 | GGAGCCAAAA GTAGAGAAGG GCTTATCTGA AAAAAGGATC TGTGGACCTG | |
| 24601 | GCTTTTATCT AATAATGCAG TGGATTCCCC CATGACATCC ATAGGAGACC | |
| 24651 | CGTAAAGTTC CTGAGACGTT TACATCCACA GAAACACTGT TAGCTTGGAT | |
| 24701 | TAAATGGAAC ACAGAGAGTA TGAAATCAAA GAAGGCTGTT GGACTCTCCA | |
| 24751 | GTTTCTACTG TTGAGATGCA GACTGGTAAA ACTACTTAGC TGCAAACACC | |
| 24801 | TGCTACCTTT AGTGAAAAGG AAGGATATCT CAGACGGTGA AACCAGAAGC | |
| 24851 | TCAAAGGGCA GTGCTAAGAG CGAAAGAGAA TTCTTCCCAG GCCTTGAAAC | |
| 24901 | CTAATGGAGT TTTCTTGGCT GGATTTTCAA ACTGCATTGG ACCATGACCT | |
| 24951 | GATTGTCCCT TTCATGTCCC CATGCTTGAG CCAGATTGTC TGCAACTGTT | |
| 25001 | ATCCTGTGCC TGTCCCACAT TTTATGTTGG GAGCAGAAAA CTTTAGTTTT | |
| 25051 | GCTGGCCCAC AGATAGAGAG AAACTGTACC CCGAGAGTTG TACTGACTGG | |
| 25101 | ACTATGCCCA GAGTCTATTT GACTCTGACT TAGATACTGT TGATTTGGGA | |
| 25151 | ATTTGAGTTG ATGCTGTAAT GAGATGAGAC TTTGGGGGAC ATTGGGATGG | |
| 25201 | AGTGAATGGA TTTTGCATTT GAAAGAGATG TGGGTTGGGT AATCCTAGCC | |
| 25251 | CACACCTGTA ATCCCAGCAC TTTGGGAGGC CGAGGCAGGC AGATCACCTG | |
| 25301 | AGGTCGGCAG TTCGAGACCA GCCTGACCAC CATGGAGAAA CCCCATCTCT | |
| 25351 | ACTAAAAATA CAAAATTAGC CAAGCATGGT AGCACATGCC TATAATCCCA | |
| 25401 | GCTACTCGGG AGGCTGAGGC AGTAGAATCG CTTGAACCCG GGAGGCAGAG | |
| 25451 | GTTGCGGTGA GCCGAGATCA CGCCATTGCA CTCCAGCCTG GGCAACAAGA | |
| 25501 | GTGAAACTCC ATCTAAAAAA AAAAAAAAAG AAAGAAAGAG ATGTGGATTT | |
| 25551 | TGGGTGGGGG ACAGAGGGAA GACCATGGTA GGCAGAATGA TCCTCTAAAG | |
| 25601 | GTGCTCTGCC CTAATCCCCA GAAGCTAAGA ATATGTTAGA TGTCAGTATT | |
| 25651 | GCGTGGCAGT AGGAATCTTA ATTAACGTTA TAGACTGTTA TGGTTTGAAT | |
| 25701 | GTCCCCTCTA AAACTCCTGT TGACATTTAA TCATCATTGT GATTGCATTA | |
| 25751 | AGAAGTGGCC CTGTTAAAAG GTGATTTAGT CCTTAAGAAC GCTGCCCCCG | |
| 25801 | TGAATAGATT AAGGTCAGTC TTGCGGGAGT GTGTTTATCA AGAATGGATT | |
| 25851 | GTTAAAAAGT GAGTTCTGGC CAGGGGCAGT GGCTTATGCC ACTCAGCACT | |
| 25901 | TTGCGGGGCC AAGACTTGAA GTCAGTTGTT TGAGACCAGC CTGGCCAACA | |
| 25951 | TGGTGAAAGT CTGTCTCTAC TAAAAAATAC AAAAAGTGTC CGGGAGTGGT | |
| 26001 | GGCGGGCGCC TGTAATCCCA GCTGCTCAGG AGGCCGAAGC AGGAGGATCG | |
| 26051 | CATGAATCCG GGAGGCAGAG GTTGCAGTGA GCTGAGATCG CCCCGTTGCA | |
| 26101 | CTCCAGCCTG GGTGATAGAG CAAGACTCTG TCTCAAAAAA ANNNNNNNNN | |
| 26151 | NNNNNNNNNN NNNNNNNNNN NNNNNNNNNN NNNNNNNNNN NAAAGAAAGA | |
| 26201 | AAGAAAAGAA AAGAAAAGTG AGTTCTGCCC TCTCTTGCTG GCTTACTCTC | |
| 26251 | ACCCTCTCTT GCCCTTCCAC CTGCCACCAT GGGATGACAC AGCACAAAGG | |
| 26301 | CCCTCACCAG ATGCCAGTGC CATGCTCTTG GACTTCCAAG TCTCCAGAAA | |
| 26351 | CATGAGCCAA ATACACTTCT GTTCATTATA AATTACCCAG CCTGTGATAT | |
| 26401 | TCTGTAATAA CAACACAAAA TAGACTGAGA CATAGATCTT CAAATAGTGA | |
| 26451 | GGTTATCCTG GATAATCCAG ATGGGCCCAA TCTAATCCCA TGAGCCTTTA | |
| 26501 | AAACTTTCTC CAGATGGAGG CAGAAGAGAA GTGGCAGAAG GGGAAGTCAG | |
| 26551 | AGAGATTTGA AGCATAAACA GGACTCCATG GTGCCGTTTC TGGTTTGACG | |
| 26601 | ATGGAGTGGT AACGTGATGA AAAATGTGGG TGCCTTCCGG AGCTGAGAGG | |
| 26651 | CTCCCACTAA CAATCGGCCA GGAAACAGGG ACCACAGCCC TACAGCCACA | |
| 26701 | AAGAACTAAG TTTTGCTGAC AACCCAAGGG GGCTTGGAAG TGTCTTCTCC | |
| 26751 | CCCATCGGTT CCAGATGTGA GACCCAGAGC GAAGGAACCA GCTGAGCCCA | |
| 26801 | CCTGGACTTC TGACCTAGAG AACTGTGAGA TAATAAGTTT GTATCATTTT | |
| 26851 | TAAGGCACTG TGTGTGTGGT AATTTGTTAT GACAGCAATA GAAAATGAAT | |
| 26901 | CCAGATGGGC AGGATCTGCC AGGCCAGTGA CATGTGGAGG GCACCCAGGC | |
| 26951 | GGATGGGATG GCATGAGAGA AGGCAGGTCA GCAATGAGCT TGCCCAGGTC | |
| 27001 | ACCTCTCCTC TCTAAGCCTC AGTTTTCCTC TCTATGAAAT GAGAGTAGTG | |
| 27051 | ATATCTCCCT CCCAGGGTCA GTGCAAGGCT GAAATAACAG ATTATAAGGT | |
| 27101 | GCTAGGTGCA CAAGAAGTGT TTGAAACATG CTAGTTGCTT TTCCATTTCC | |
| 27151 | AAGAGAGCTC TCTGGTCTTG GGGGATGGAG GCAGTGCGGC CCCTCGGGAT | |
| 27201 | TACTGACAGG TCCTGCTCTG TTTCTGCAGT GGAGCCGGCC CCACCTGTCC | |
| 27251 | TGGTGCTCAC CCAGACGGAG GAGATCCTGA GTGCCAATGC CACGTACCAG | |
| 27301 | CTGCCCCCCT GCATGCCCCC ACTGGATCTG AAGTATGAGG TGGCATTCTG | |
| 27351 | GAAGGAGGGG GCCGGAAACA AGGTGGGAAG CTCCTTTCCT GCCCCCAGGC | |
| 27401 | TAGGCCCGCT CCTCCACCCC TTCTTACTCA GGTTCTTCTC ACCCTCCCAG | |
| 27451 | CCTGCTCCTG CACCCCTCCT CCAGGAAGTC TTCCCTGTAC ACTCCTGACT | |
| 27501 | TCTGGCAGTC AGCCCTAATA AAATCTGATC AAAGTATGAT GACCTACAGG | |
| 27551 | AGGCCTGCTT GCCAAGTCAA CAGATTCAGT ACAGAAAAAC TGAAAAATAC | |
| 27601 | AGATAAGCTC TAAGAAGCAG ACCAAAAGTA CCCAGAGATG ACCGCACATC | |
| 27651 | ACTCTGGTGT ATATCCAATT TCAGATTTGT TTTCTGTGTA TGCATGTGTG | |
| 27701 | TATAGCTGCA TTTATTTATG GCAAGGGCTG GCAGACTTTC CCGAAGAAGG | |
| 27751 | CCAGATAGTC GATATGTTTG GCTTCATGGG CCGTATGTTC GCTCAGGACT | |
| 27801 | ACTCAACGCT GCAGTTATAG CACAAAAGGA GCCGTAGCCT ATACGTAAAT | |
| 27851 | GAATGGGCAT CGCTGGGTTC CAGTAAAACT GTTTACAGGC CAGGTGCGGT | |
| 27901 | GGCTCATGCC TGTAATCTCA GTACTTTGGG AGGCCGAGGT GGTGGGAGGA | |
| 27951 | TTACCTTAGC CCAGGAGTTC AAGACCAGCC TGGGGAACAT GGTGAAACAT | |
| 28001 | TATCCCTACA AAAAAAAAAA AAGCTGGGTG TGGTGATGCA TGCTTGTGGT | |
| 28051 | CCCAGCTGCT TGGGATGCTG AGGCAGGAGG ATCGCTCGAG CCCAGGAAGC | |
| 28101 | AAGGCCACAG TGAGCCATGA TCGCACCACT GCACTTTAGT CTGGGCAACA | |
| 28151 | GAGTGAGACC TTGTCTCAAA AAAAACAAAA AATAAAACTT TTTACATAAA | |
| 28201 | CAAGTGGCCA ACCAGACTTG GTCCCTGGGC CTCTGCTCTT GAATGTTCTT | |
| 28251 | GCTTCCACTA AAGTAACATT CACACTCCCG ATTTTTGCAT ACTCTGGGTT | |
| 28301 | CTGGGGAATA TAGATCCGAA TCCAGCGTGG TTCCTGCCTT CAAGAACCTC | |
| 28351 | ACAAATATTC TAGACCAGCA CTGCCCAATA GAAAGAAATA TAATGCAAGC | |
| 28401 | CACATGTGCA GTTTTAAGTG TTCCATGTTA AATTAAGTAA AAAGAGACGG | |
| 28451 | GTAAATCGAA TTTTAATAAC AGATTTTACT TCATCCAATT GAATGGTATC | |
| 28501 | ATTTCAATGA GCAATTCTGA TAGTGATTGA GATCTTTTAC ATTCTTTTTC | |
| 28551 | ACTACGTCTT TAAAATCTGA TGTGTGTTTT GTACTTGGAA CACTTCTCAG | |
| 28601 | TGTGGACCAG ATGCATTTCA CATACTCAGT AGTCACGCGT GGCCAGTGCC | |
| 28651 | TTCCATACCA CACAGTGCAG CATCTGTAGA GGTTTCCTCC ACTGCTGATA | |
| 28701 | GACTAGGAGA CCCCAAGATG GAAAGCCTGA AGAATCTGCT CCTTGAAGTA | |
| 28751 | GGGACCTTAA TGGGGTGCAC GCCAGGGCGA CCCCAAGTGG TAGGCTGCTT | |
| 28801 | TTGAACCATG GCTATCCCTA CCTCTAGACT CAGCTGAAAA GAACTCAGGT | |
| 28851 | AGTCTTGGGA AGTGCTTCCT CAATGCTTAA ACTTTAATGC AGGAAAAGAA | |
| 28901 | TAGAAAGTTC AGGCAAGGAG GGAGGATCAC TTGAGGCTGG GAGTTCGAGA | |
| 28951 | CCAGCCTGGG CAACAGCAAG ACCTTGCCTA TACAAAAAAT AATTTTAAAA | |
| 29001 | AATTACCCAG GTATGGTGGT GTGGATCTGT AGTCCCTAGT TACTTGGAGA | |
| 29051 | GCTGAGGTAG GAGGATCGCT TGAGCCCAGG AGTTTGAGGC TGCAGTGAGC | |
| 29101 | TGTGATCACA CCACTGCACT TTGGCCTGGG TGACAGAACC AAACCCTATC | |
| 29151 | CCCTACAAAA AAACAAAAAA AAAAAACAAA AAAAAACACC CTACCATGTC | |
| 29201 | TGCCAACCCC ACTCTGTCCT GGCTGTGTGA AACCAGTCCC CACAGCAGCT | |
| 29251 | CTGCCACTCT CTGCTTCTTT TCCAAACAGA CCCTATTTCC AGTCACTCCC | |
| 29301 | CATGGCCAGC CAGTCCAGAT CACTCTCCAG CCAGCTGCCA GCGAACACCA | |
| 29351 | CTGCCTCAGT GCCAGAACCA TCTACACGTT CAGTGTCCCG AAATACAGCA | |
| 29401 | AGTTCTCTAA GCCCACCTGC TTCTTGCTGG AGGTCCCAGG TGGGTATCAA | |
| 29451 | GTGGTGCAGA AGGAGAAACT TTCCCTCTGG GCCTTGGGAG CTTCGTGACA | |
| 29501 | CAGTGGTTAA GAACATGAGC CTAGAGATAG ACTCGCCTGG ATTAAAACCA | |
| 29551 | CACTCATTGT GTGTCTTTGG GCAGCTTACA TAATGCCCCG AACCTTGGTT | |
| 29601 | TGCACAGTCT GCAGGATGGG TTTATTCTTG TGAGGATTAA ATAGGGTCAT | |
| 29651 | GTATGTGAAG CACTCGGCAC AGGTGCAGTT GTAGACAAGA GCCATTGTTG | |
| 29701 | TTTCTCTCAT TGTTATTTTT CCTTCCTTAG AAGCCAACTG GGCTTTCCTG | |
| 29751 | GTGCTGCCAT CGCTTCTGAT ACTGCTGTTA GTAATTGCCG CAGGGGGTGT | |
| 29801 | GATCTGGAAG ACCCTCATGG GGAACCCCTG GTTTCAGCGG GCAAAGATGC | |
| 29851 | CACGGGCCCT GGTATAGCAA ATCTGGGGGT GTGCGGCAGG TGGGGAGGGG | |
| 29901 | TTGAGAGTAA GGGAGTGGGG CTGGAGCTAT GAGTTGTTCA GATAGAATAT | |
| 29951 | CAAGATGGTC CAGACTCTTG GACCAAAACA TCTATCTTTG TGTCTGAATT | |
| 30001 | TCCACCATTA GTAATGCATT CATTTAGTCC TGAATAAAAT GGCAAACAGG | |
| 30051 | CCCTGGAGGG AGCAGTGCCT TAAGTTCCTT TGAGATAAAT AACTTCACCT | |
| 30101 | CTGCTAAGGA TGTGTCAGCT GCTGAGAGCA GAGCCCCTGG CCTTGGACCT | |
| 30151 | CAGGAGAGAC ACTCAAAAGG GGAGGAGAGG AGGCACCAAA GGGGACATCT | |
| 30201 | TAAAAGAGTT CCAATTTTTA GTTCACACTT TAACCCAGGA TAAGCTGTGT | |
| 30251 | CCTGGCTGAC CTTGGAGTTT CTTCCCTGGT CTGCTGGGTC TCTCCCTTAG | |
| 30301 | AACCTAGGGG CGAGCTGGGG CAGGGGAAGC CCAGGAGGTG ATATAGGTCG | |
| 30351 | GCCCTGTTCA GATGAGGGCT GGCAGGGGCA GCTTGGGCAT ATGCGAGGCT | |
| 30401 | CCGATGGGCA TGGGGGCTTT GAGGATGGAT TCTGAGTGTC CCTGCATCGT | |
| 30451 | GGCAGGGTGG CAAAGGGAGC ATTTCCAAAT TTCCTGGCTC CAGGATCTGT | |
| 30501 | GGGAGAATCC CACTAACTGT CAGGGTGACA ACCTCGGGTA GACATGTCTG | |
| 30551 | TGCCCTGCCC CGTGCCCTCA GCCTTCCTGT TAAGAGCACA CCAGCTGGAT | |
| 30601 | TTGCAACTCC CAGCGCCTGC ACCCAATGGG CTTTCTCTGG CCTCTGGAGC | |
| 30651 | CCACATTGCC CCTGCATGTG GCAGGCTGCA AGTGTCACAG CCACCAGCTC | |
| 30701 | TTCCATTCCT CAACAATGAC TGTGGGTAAA TAGCCCAGGA GCGTCCCCCT | |
| 30751 | CCTGGGATGG TTCTGAGGTG CGTGTGCCCA GTGGCTCCCT GAGTTGCCAG | |
| 30801 | CAGGATTAAG TGCCAGTAGC CCTAGTGGTC AGCTGCTTGA TAACACCCTG | |
| 30851 | CTTCCTGGCT GCTCCCCCAG TCCCATCTGG TGTGTTCTGG GATCATCTCC | |
| 30901 | CAAAGAAACT GCTTACACTT GAAGCCTTGT CTGAGGTCTG TTTCTAGGGG | |
| 30951 | AATTCAGATG ACGATAATTA TGCTTCAGGA AAGCCTPAAT TTTCTGCTTT | |
| 31001 | TCTCTCCCCT ACCCAAATCA GGACTTTTCT GGACACACAC ACCCTGTGGC | |
| 31051 | AACCTTTCAG CCCAGCAGAC CAGAGTCCGT GAATGACTTG TTCCTCTGTC | |
| 31101 | CCCAAAAGGA ACTGACCAGA GGGGTCAGGC CGACGCCTCG AGTCAGGGCC | |
| 31151 | CCAGCCACCC AACAGACAAG ATGGAAGAAG GACCTTGCAG AGGACGAAGA | |
| 31201 | GGAGGAGGAT GAGGAGGACA CAGAAGATGG CGTCAGCTTC CAGCCCTACA | |
| 31251 | TTGAACCACC TTCTTTCCTG GGGCAAGAGC ACCAGGCTCC AGGGCACTCG | |
| 31301 | GAGGCTGGTG GGGTGGACTC AGGGAGGCCC AGGGCTCCTC TGGTCCCAAG | |
| 31351 | CGAAGGCTCC TCTGCTTGGG ATTCTTCAGA CAGAAGCTGG GCCAGCACTG | |
| 31401 | TGGACTCCTC CTGGGACAGG GCTGGGTCCT CTGGCTATTT GGCTGAGAAG | |
| 31451 | GGGCCAGGCC AAGGGCCGGG TGGGGATGGG CACCAAGAAT CTCTCCCACC | |
| 31501 | ACCTGAATTC TCCAAGGACT CGGGTTTCCT GGAAGAGCTC CCAGAAGATA | |
| 31551 | ACCTCTCCTC CTGGGCCACC TGGGGCACCT TACCACCGGA GCCGAATCTG | |
| 31601 | GTCCCTGGGG GACCCCCAGT TTCTCTTCAG ACACTGACCT TCTGCTGGGA | |
| 31651 | AAGCAGCCCT GAGGAGGAAG AGGAGGCGAG GGAATCAGAA ATTGAGGACA | |
| 31701 | GCGATGCGGG CAGCTGGGGG GCTGAGAGCA CCCAGAGGAC CGAGGACAGG | |
| 31751 | GGCCGGACAT TGGGGCATTA CATGGCCAGG TGAGCTGTCC CCCGACATCC | |
| 31801 | CACCGAATCT GATGCTGCTG CTGCCTTTGC AAGGACTACT GGGCTTCCCA | |
| 31851 | AGAAACTCAA GAGCCTCCGT ACCTCCCCTG GGCGGCGGAG GGGCATTGCA | |
| 31901 | CTTCCGGGAA GCCCACCTAG CGGCTGTTTG CCTGTCGGGC TGAGCAATAA | |
| 31951 | GATGCCCCTC CCTCCTGTGA CCCGCCCTCT TTAGGCTGAG CTATAAGAGG | |
| 32001 | GGTGGACACA GGGTGGGCTG AGGTCAGAGG TTGGTGGGGT GTCATCACCC | |
| 32051 | CCATTGTCCC TAGGGTGACA GGCCAGGGGG AAAAATTATC CCCGGACAAC | |
| 32101 | ATGAAACAGG TGAGGTCAGG TCACTGCGGA CATCAAGGGC GGACACCACC | |
| 32151 | AAGGGGCCCT CTGGAACTTG AGACCACTGG AGGCACACCT GCTATACCTC | |
| 32201 | ATGCCTTTCC CAGCAGCCAC TGAACTCCCC CATCCCAGGG CTCAGCCTCC | |
| 32251 | TGATTCATGG GTCCCCTAGT TAGGCCCAGA TAAAAATCCA GTTGGCTGAG | |
| 32301 | GGTTTTGGAT GGGAAGGGAA GGGTGGCTGT CCTCAAATCC TGGTCTTTGG | |
| 32351 | AGTCATGGCA CTGTACGGTT TTAGTGTCAG ACAGACCGGG GTTCAAATCC | |
| 32401 | CAGCTCTGCT CTTCACTGGT TGTATGATCT TGGGGAAGAC ATCTTCCTTC | |
| 32451 | TCTGCCTCGG CTTCCTCATC TGCAGCTACG CCTGGGTGTG GTGAGGGTTC | |
| 32501 | TAGGGGATCT CAGATGTGTG TAGCACGGAG CCTGCTGTGT CCTGGGTGCT | |
| 32551 | CTCTACGTGG TGGCCGGTAG AATTCTCCAT CTATCCAGGC TCCAGGAGAC | |
| 32601 | CCCTGGGCAT CTCCCACCTG TGGCCCCTAA ACCCAGAGTG ACTGAGAGCA | |
| 32651 | CTTACCATTC AGCTTGTCTC ATCCCCAGTC TACCTCCTTC CTTCTACCCT | |
| 32701 | CACTGCCTCC CAGTCAGGAG AGTGAGCTCT CAGAAGCCAG AGCCCCACCC | |
| 32751 | AAGGGGACCC TGGTCTCTCC GCCTTCACCT AGCAATGGGA ACCCTGCTTC | |
| 32801 | CCAGGGGAGG AACCAACTGC TCCACCTTCT AGGGACCCAG TTTGTTGGAG | |
| 32851 | TAGGACACTA ACATGGCAGG AATCGGACTT CTGGGCCTGT AATCCCAGTT | |
| 32901 | TGGATGGCAC GTTAGACTCT TGGTTGACCG TTGTGGTCCT TAGAAGTCCC | |
| 32951 | ATTCTCCCTT CCAGTTATGA GAAACCAATG CCTTCTAGAT TCAGGTGACT | |
| 33001 | ATCCTTACCT GGGGGTGCTG ATGCATCCTC AGTTAACCTA CACCCACCTG | |
| 33051 | AATATAGATG AGCGTAGCTG AGTTTTCACC CGTAGGACCG AAGTGTTTTG | |
| 33101 | TGGTGGAGTA TCTGAACAAC CTTGGCTCTG TGGCCATTCA ACCTGCCAGG | |
| 33151 | ACTAACATTT CTGGATTTGT GAAGAAGGGA TCTTCAAAGC CATTGAACCC | |
| 33201 | ACAGAGCTGT GTTGCTTTAA AGCCACCACA AGGGTACAGC ATTAAATGGC | |
| 33251 | AGAACTGGAA AAGCTTCTTA GGGCATCTCA TCCAGGGATT CTCAAACCAT | |
| 33301 | GTCCCCCAGA GGCCTTGGGC TGCAGTTGCA GGGGGCGCCA TGGGGCTATA | |
| 33351 | GGAGCCTCCC ACTTTCACCA GAGCAGCCTC ACTGTGCCCT GATTCACACA | |
| 33401 | CTGTGGCTTT CCACGTGAGG TTTTGTTTAG AGGGATCCAC TACTCAAGAA | |
| 33451 | AAAGTTAGCA AACCACTCCT TTTGTTGCAA AGGAGCTGAG GTCAAGGGTG | |
| 33501 | GCAAAGGCAC TTGTCCAAGG TCGCCCAGCA GTGCTGCTCT GATGACTTGT | |
| 33551 | GCACATCCCC AAGGGTAAGA GCTTCGATCT CTGCACAGCC GGGCCAACCT | |
| 33601 | CTGACCCCTT GTCCATGTCA GTAAAATATG AAGGTCACAG CCAGGATTTC | |
| 33651 | TAAGGGTCAG GAGGCCTTCA CCGCTGCTGG GGCACACACA CACACATGCA | |
| 33701 | TACACACATA CGACACACAC CTGTGTCTCC CCAGGGGTTT TCCCTGCAGT | |
| 33751 | GAGGCTTGTC CAGATGATTG AGCCCAGGAG AGGAAGAACA AACAAACTAC | |
| 33801 | GGAGCTGGGG AGGGCTGTGG CTTGGGGCCA GCTCCCAGGG AAATTCCCAG | |
| 33851 | ACCTGTACCG ATGTTCTCTC TGGCACCAGC CGAGCTGCTT CGTGGAGGTA | |
| 33901 | ACTTCAAAAA AGTAAAAGCT ATCATCAGCA TCATCTTAGA CTTGTATGAA | |
| 33951 | ATAACCACTC CGTTTCTATT CTTAAACCTT ACCATTTTTG TTTTGTTTTG | |
| 34001 | TTTTTTTGAG TCGGAGTTTT GTTCTTGTTG CCTAGGCTGG AGTGCAGTGG | |
| 34051 | TGCGATCTCG GCTCACTGCA ACCTCCACCT CCCGGGTTCA AGTGATTCTC | |
| 34101 | CTGCCTCAGC CTCCCAAGTA GCTGGGATTA CAGGCACCCG CCACCACACC | |
| 34151 | TGGCTAATTT TTTTGTATTT TTAGTAGAGA TGGGGTTTCA CCATGTTGGC | |
| 34201 | CAGGCTGGTC TCGAACTCCT GACCTCAGGT GATCCGCCCG CCTCGGCCTC | |
| 34251 | CCAAAGTGCT GGGATTACAG GCGTGAGCCA CCGCGCCCAG CCAAACCTTA | |
| 34301 | CTATTTTTTT AAAGAATTTT TTCCAGAGTT TAATTTCTGA CATAGCTTAA | |
| 34351 | GTTTTCCAGT AACTCTAAAC TCCATCTCCT TTATCGTCAT TAAGTCATTC | |
| 34401 | ACAAAAAGCC AGGAGAAGCA TTTGGAAAGG GCATGATAAT CAGTATAATA | |

Table 5 presents a correlation between the genomic sequence shown in Table 4 and the location of the corresponding regions of the cDNA sequence shown in Table 1.

TABLE 5

| Region in Genomic | Sequence | | Corresponding Region |
|---|---|---|---|
| Sequence | Attribute | Length | in cDNA sequence |
| 1-2001 | 5′ sequence | 2001 | — |
| 2002-2059 | Exon #1 | 58 | 1-58 |
| 2060-8326 | Intron #1 | 6267 | — |
| 8327-8450 | Exon #2 | 124 | 59-182 |
| 8251-19513 | Intron #2 | 11263 | — |
| 19514-19698 | Exon #3 | 185 | 183-367 |
| 19699-27229 | Intron #3 | 7531 | — |
| 27230-27372 | Exon #4 | 143 | 368-510 |
| 27373-29279 | Intron #4 | 1907 | — |
| 29280-29439 | Exon #5 | 160 | 511-670 |
| 29440-29730 | Intron #5 | 291 | — |
| 29731-29861 | Exon #6 | 131 | 671-801 |
| 29862-31021 | Intron #6 | 1160 | — |
| 31022-31780 | Exon #7 | 759 | 802-1560 |
| 31781-31783 | Stop | 3 | 1561-1563 |
| 31784-34450 | 3′ sequence | 2667 | — |

Several sequence polymorphisms have been identified in the sequence shown in Table 4. These are summarized in the Table 6:

TABLE 6

| SNP | Position | SNP Changes | |
|---|---|---|---|
| variation | 30962 | allele = “A” | allele = “G” |
| variation | 30655 | allele = “A” | allele = “G” |
| variation | 28744 | allele = “A” | allele = “G” |
| variation | 28448 | allele = “C” | allele = “T” |
| variation | 9426 | allele = “A” | allele = “G” |
| variation | 9162 | allele = “A” | allele = “G” |
| variation | 8811 | allele = “C” | allele = “T” |

A CRF2-encoding nucleic acid is also present in the genomic nucleic acid sequence shown in Table 7:

TABLE 7

| | |
|---|---|
| AGGAAGGAAGGAAGGAAGGAAGGAAGGAAGGAAAGAAAGAAAGAAAGAAAGAAAGAAAGA | (SEQ ID NO:22) |
| AAGAAAGAAAGAAAGAAAGAAAGAAAGAAAGAAAGAGAAAGGAAGGAAGGAAGGAGAAAA | |
| GAAAGTCAACAGTCAACATTTCAGAGATCCCAAGATACCAACACTGACCGTGCCTGCTGC | |
| TCTTCCATCCTCCTCCACCCTGCGCCTTTGAGGTGGAATTGCGTCCTCTGTGAGCAGGGC | |
| TTTGTTAAGAGATCCTAATTAAGGCCAGGCACAGTGGCTCATGCCTGTAATCCCAGCACT | |
| TTGGGAGGCTGAGGTCACCTGAGGTCAGGAGTTCAAGACCAGCCTGCCCAACATGGTGAA | |
| ACCCCATCTCTACAAAAATTAGCTGAGCATGATGGCAGGTGCCTGTAATCCCAACTACTT | |
| GGGAGGCTGAAGTGAGAAAATAGCTTGAACCCAGGAGGCGGGGTTGCAGTGAGCCAAGAT | |
| CACACTATTGCATTCCAGCCTGGGCGACAGAGCTTTTGTCTAAAAAAAAAAAAAGAAAAA | |
| AAATCCTGATTAAGCAGAAGCCTTGATGCTAGTCCCAGAAGCATCCTGAAATTTCCAAAA | |
| GAAATTTCCCCCGCGGTTAAACTCAGAGCAACTTTTGGACCCACCAAGCTCTGTGAAAAT | |
| CATTTTCTCTTCCAAAAACTGATGGGACCAAAGCTGATCCCAGTTTCAAATAATTATCAA | |
| AAAATTGGAAACGAAATATGATCAGAAAAGAAGAAAGTTGAAAAAGAAAATCCTCATCAC | |
| CCAAAGACAACAACCATTAATATTTTGGTAATTATTATTCCAAATATCTTTCTATGCATA | |
| CAGACAGACTGACACACACACACACACACACACACACACACACACACACACTTTTTTTTT | |
| TTTTTTGAAACTGAGTTTCACTCTGTCGCCCAGGCTGGAGTGCAGTGGCGCGATCTCGGC | |
| TCACTGCAACCTCCGCCTCCTGGGTTCAAGCGATTCTCCTGCCTCAGCCTCCCTGATAGC | |
| TGGGATTACAGGTGAATGCCACCACGCCCGGCTGATTTTCTGTATTTTTAGTAGAGACGG | |
| GGTTTCACCATGTTGGCCAGGCTTGTCTCCAACTCCTGACCTCAGGCGATCCACCCGCCT | |
| CACCCTCCCAAAGTGCTGGGATTACAGGCGTGAGCCACCGCGCCCGGCTACACACACACT | |
| TTTTTAATGGGCCTATGTTTTAGCACTCGCTTTTCTGTTTCTCAGTGTGTTGCAAACACC | |
| TCGGTGTCGATACACACCATTCGGCAACGTCCTCCTAAAGGGCCGCATAATATTGCGCGT | |
| CGTGGCGTGTGCCTTACTGGGAAGCTACTGCTGTCCAGGTGAACACCACAGCCTTCGGGG | |
| TCAGAAAGACAGCTTTCCCCAGAACAAGCACCTGAAGCTCTGGGGCCTGCCGCTCCCCGG | |
| GAGAGAAGTACGTGGAGAAGGGCAGCACGGATCCGCCGGGATCCCCGGGGGCATTAAAGG | |
| GAATCGCGTGTGTAAGGCGCGGAGCTCAGCATCCGGCTCAGAAACGCGCTCGGATCCCGC | |
| CAATGGCATTGAGGCCGCGTAGCCAAACCGGCCTTGAACTCTCCCTAATCCTGCCAAAAT | |
| GGCCCGTCCTGGAGCACTGGACTGGCCGTGGGTTATTGATCATCAGCCGGTTTCTTCCCC | |
| TCCCCTGCCCTTCCCCCGTGCACGGATTTACTGATTTTTTTTTCCGGGAATTGAGTAAAA | |
| CAAAACTAAGTGCAGATGAAGCAGAGGTACGGGCGAGTTTCGAGCGCGGGGACCGGCGCG | |
| CTCCCCCCCCCCCCTCCCCCCGCGGCGGGGCTGTCCCCAGGGACCTTCTCAGTGAATCCT | |
| AGGCGGCAGGGACGGGCCCGCGGCTCTGCGGGCCATTGGCTGCCGACTGCGTCACCTGCC | |
| CGCGGTGGGCTAGGAGACGGGAGGCGGGAGGCGGGAGGCGGGGACCTGGGTCCGGGCGGG | |
| GACGCCGCGGCAGGAAGGCCATGGCGGGGCCCGAGCGCTGGGGCCCCCTGCTCCTGTGCC | |
| TGCTGCAGGCCGCTCCAGGTAAGGGCGCGGGGCCGCGGGAGGGAGGGGGAAGAGGGCTCC | |
| CCGGGCCGGGCCGCGCCTACCCTCGGACCCGGAGCTCCTGGGACAGGCACGGGGTCCGCA | |
| GCCACCCGAGCCGGGTGCGAATCGGCCCTGCCTACGCGCCCCCAGTTTGCTTCTTCCCAG | |
| GACTGAACAGAACCGGGTCTTTGATATTCCTCTCCCGCAGGAAACGAATCCAGTTTCCTA | |
| ATGCTTCCAGCTTCAGGAGAACTGGAGAAAAAAGACAGCGGCAGTTTGATACTGCATATT | |
| TTTTAATAAAGTGCTTTTTAATGTTTCCTAAAGAAAGCACTGATCCCTGCGTGAAAACCA | |
| CACTTGACCCTAAAGTGTGGACAGCAGGGAAAGTGGGACCGATTGATGTCCCTTCCCGTT | |
| CCTGCCAGGCCTCTGGTGGGACGGAGCTCTGGTCGCCTGTGCCCTGCTTTCTAACAAGAC | |
| GGCTTTCTTTTGGTGGTGGTTGTTGTTTTGTTGTTGTTTTGTTGTTGTTGTTGTTGTTGT | |
| TGTTTTCCCACCTCTACTGATGAGTAAGGTGTCAGGTACAAAATTCCTCGCCGTAGGACC | |
| CAACCACCAAACCTCACCGCCCACGACTCCAACCGAAGCAGGGAAGAGAAGGTCCAGAAA | |
| TCGCCCCCAGGATATTTTCCTAGTCTTGGACTCACAGTTTAAAGAGCTGTAAAGGTCCCT | |
| GGGCATAATCCAATCATCATAAAAGCCTATATTTATTCAGCAACTTCTTTGTGCCAGGCA | |
| CCGCATTATTCTGGAAGCCTCACGACCCAGCCATCCTAGGAGGTAGATATTATTTTTACT | |
| TTTCCGATGGGAAAACTGAGGCTCAGAGCAATTCAGGGAATTCCTCAAGAAGGACGGCAG | |
| AGGTGAGGCACACAGAAGAGAGAAGAGGGGCTAAAGCAAGCCTGGCTAGCTTTTGCCTCC | |
| AGGGTAGGCACGTGGGACAGGCTGTCCATCCACTGGGTCACTAGGCCAGCCAGGGATGCT | |
| CCAGCCCCCAGTGCCCACAGCAGCGTTCTCTGTGGCTQATGAGGGACCGTGTACCTGTGT | |
| GTGGAGGGAGGGTGGGGTCTTCTGTTCCCCTTTCACTGTCAAGCCCAGACCTTCTTGTAC | |
| TTTCACCTGATAAGTATTTAATATACACAACACTAACTATGGTGTGATGATTTAGGAGTA | |
| AGTACAGCCAGATCTAAGTTCAAATACTGGCTCCCACACAAACTGACTGTGTAGCCTCAG | |
| GCAAGTTAGTTAGCATCTGTCTCTGAGCCTAGCGCCCTTTCCATGGAAGCAGAATGAATG | |
| ACACCTACCCCATAGGGTGGTCTGTCCCAAGGGTGATTGAGGTTTTACATGTAAAGAGCC | |
| AAACTAGTGCCTGGCATCCTTTGAAGGCTTCATAGAGGAAAGTTGCTCTAGCTGCTGTTT | |
| TTCTCATGTGACCTAGCTCGAATCTGGGGACTGTCCTGCCCATAGGATACCTTACAAGTG | |
| GCTTGCAGACAGCCTGGTCTCCTGCTGGTCACCCGTTAGGAAGTCCAGAAGCTGGGAGTA | |
| GTAATAGCACTAGCCTCGTGGTGATACAGTCCCAGCTAGAGGACACAGGATGAGGTGGAA | |
| GCAGGCACCCACTTTTGGGTCTAAAAGGTGATGGGTAGGCAGCCGAGGCTGGGGACAGCC | |
| ATCCACAGAACTGGACCCTCCCTCCCTGATGCCATTTTGCAACCCGTATGGATTTCCATC | |
| ATGGCACATGGGACACTTCAGGACCCTGAATTCTCCATGGGACCATGAGCTCCTATAGGG | |
| CAGGAATGAAGTTGTGTTCTTCTTTGAAACCCCTGGCACACCGTGGTCAACAGATCTTGT | |
| TTGACTCGTAGTGGTCAATAGATGGAATAGTTGGAATCATAAACCTCAATAGACCCCATG | |
| AGAACCTAGAAGACAAAGTACAGTCAAGAGCTCGGACTTTGGAGTTGGCTAGGCCTGGAC | |
| TGAATCTGATTCTACAACTTAATAGCTGAGAGGGCCTTGGTTTTCCCATCTGTAAAGATT | |
| ATAATTATTATAATGAATACCTACCTCCTAGGGATGTAATGAGGATTAAAAGAGAAAGTG | |
| CAGGTAAACTGTTTAACACAGAACCTGGCTCATAGAACACAATACACATTAGCTGCTATT | |
| ATTATTATTATTATTTTATTTATTTATTTTGAGACAGAGTCTCACTCTGTCACCCAGGCT | |
| GGAGTGCAGTGGCGCAATCTCGGCTCACTGCAACCTCCACCTATCGGGTTCAAGCAATTC | |
| TCGTGTCTCAGCCTCCCAAGTAGCTGAGATGACAGGCGTGTGCCACCATGCCCAACTAAT | |
| TTTTGTATTTTTAGAAGAGACGTGGTTTCACCATGTTGGCCAGGCTGGTCTCAAACTCCT | |
| GACCTCAGGTGATTTGCCTACCTCTGCCTCCCAAAATGCTGGGATCACAGGGGTGAGTTA | |
| CCATGCCCGGCCTTAGCTGCTATTATTATCATCATCGTTATCATCATCATCATCACCTCG | |
| TAGATATGTCAAGGAAGATTCCCTGGAGGAAGTGACATTTGAATCAAGTATTTCAAAGAC | |
| TAGATGGTGAATACCAGGCAGTCAAAGACACCTGGGTTTAAAAACATCCAGAAGAATGCA | |
| GTGGCTTGGCAACATCGAGCAGGAAGATTGCCTGATGAGCCTGTAGGGTAGCTGTTGGGG | |
| AGAGAGCAGCAAGACGGCCTGGCCAGGCCAGGCCAGGCCACGTCAGGCAGGGCCTCACAA | |
| ACCTCAATAACAAATGTGGACTTTATTCTGAGGCCAAGGAAAGGGCATGAAACTGGGGAG | |
| TGGTGTAATCAGATGCGTATTTCAGAAGATGAAGATTAACAGTGAGAAGGAAAATGTGCC | |
| ACAGAGGGGAATAGAGGTCAGTTAAAGGGAGTCAGGGAAAGTGTCCTCGAGACAGTGACA | |
| TCAAAGGAATGTGAAAACAGCAAAGGAGTGAGCCAGGTGGATATCCAGGGGCAGAACTGT | |
| TAAGGCAGAGGCAACAGCATGAGGGAACAGCGTGTGCAAAGGCCTGGAGTTGGGAGTGTG | |
| GCTGGGGTGCTCCAGGAAGGGCAAAAAGTCCTGTGTGGATGGAGATATGGGAGCAAGGGA | |
| GGAGTGGTGGGTCAGATTGGGTAGGGCCTTGGTGGTGATTGTAAAGACTCTGGAGTTTAG | |
| ACCAGGCACAGTGGCTCAGGCCTGTAATCCCAGCACTTTGAGAGGCCAAGGTGGGCGGAT | |
| CACCTGAGGTCAGGAGTTCGAGACCAGCCTAGCCAACATGGTGAAACCTCGTCTCAACTA | |
| AAAATACCCAAATTAACCAGGTGTGGTGGCACAAACCTGTAATCCCAGCTACTCTGGAGG | |
| CTGAGGCAGGAGAATCGCTTGAACCCGGAAGGTGGAGGTTGCAGTGAGCTGAGATTGTGC | |
| CACTGTACTCCAGCCTGGGTGGCAGCATAAGACTCTGCCTCAAAATAAAATAAAAATAAT | |
| AAAGACTTTTGAGTTTCCCTGGAGTGAGAGGAAAGCCTTAGAGGGCTTTAGCAGGAGATG | |
| AACATGATCTGATTTTCATTTTTAATCCTTCCTGCTATGTGGAGAATGGACTGAAGGCAA | |
| GGTGTTTTGTATATTTGTCTGTTTCGTAGAGACAGGGTCTTGCTCTGTTGGCCAGACTGA | |
| AGTGCAGTGGCACAATCACGGCAGCCTTGAACTCCTGGGCTCAGGCGAAACTCCCACCTC | |
| AGCCTCCTTACTCTCACCATTGTGCCCTGCTAATTTTTTAAAAAATTTATTTTGTAGAGA | |
| TGTGGTCTCACTATGTTGCCTAGGCAAGTCTTAAATTCCTGGTCTCAAATGATTCTCCTG | |
| CCTCGATGTCCCAAAGTGCTGGGATTACAGGTGTCAGCTGCCATGCCCGACCTGTATTTT | |
| TTTTTTTAATGGGGAAAAAGCCTTTTAATAGTATGAGGTGTTTTCTGGTGTTTCTACCAT | |
| AAAGCTCTTCTGTAAATCAAAATGAGAATGTAATTATTGATAGAGCAATGACCTTAGACT | |
| ACAGTGCAGACTTTTCATCTTACATTTGGGCTCATGAATTTTAGTATAACTGATTATGAC | |
| AGTGTTTTTTACATAGTTATGATCTAGAGCAGAACTGAAAACAAAATAACACATACTCTA | |
| CATCAATATATTCGTTCAGTAATATCTGGGCTTGGATGAACCTGCAGAAGTAGGTAAAGC | |
| TGTCAGATATTTTCTTAAACCAACAGAAAAGAAATGTATATGACAGATGTTGTGTTTACT | |
| TACTTATTTATTTATTTATTTATTTATTTGAGATGGAGTCTCACTGTGTCACCAGGCTGG | |
| AGTACAGTGGTGTGATCTCTGCTCACTGCAACCTCCACCTCCCGGATTCAAGCGATTCTC | |
| CTGCCTCAGCCTCCTGAGTAGCTGGGATTACAGGCGTGCACCACCACGCCTGGCTAATTT | |
| TTGTGTTTTTAGTAGAGACAGGGTTTCACCATGTTGGTCAGGCTGGTCTCGAACTCCTGA | |
| CCTCGGGATCTGCCCACATCAGCCTCCCAAAGTACTGGGATTACAGGCATGAACCACCAC | |
| GCCCAGCCTGTATTTATTTTTTTACCACTATGGAGTCCAATATGAAATTCTCACAACTAT | |
| GCATATACATTATTAACATGTAAGCACACCTAGGTATAAATATGCACATAGTCCATTAAT | |
| TACATCAGGGGAATTAAAAACATACTTTCAAGTTAAAATGAATTTTCAGGAAAAAAACTG | |
| CATTCACAAATCTGAAATGTGAATACAAAAATGAAATTGTGAAATAAATAATGAATATAG | |
| GTGTCACCTAAACTTCCATAGTAACATGCCTCCAAATGTGGATTTAGTGATCATCCACCT | |
| TGGGACAAGGGCTTTTGAGAGCCTCCAGCTAAATTAGGGTTCCAGTAGCAGAGTGGCTGG | |
| CAAGCCTGCCCTAATGAATAATGCCAGCGAGCTGGGCGTGGGTACTTACAGTGTGCCCTT | |
| CATGGAATACTTTTTTTTTTTTTTTTGGAATGGAGTCTCGCCCTGTTGCCCAGGCTGGAA | |
| TGCAGTGGCACAATCTCAGCTCACTGCAACCTCGTCCTCCTGGGTTCAAGCAATTCTCGT | |
| GCCTCAGCCTCCCAGGTAGCTGAGACTACAGCCCTGTGCCATCATGTTCTGCTAATTTTT | |
| GCATTTTTAGTAGAGACGAGGTTTCACCAAGTTGGCCAAGACTGGTCTTGAATTCCTGAC | |
| CTCAGGTGATCTGCCCACCTTGACCTCCCAAAGTGCTGGGATTACAGGCTTGAGCCACTG | |
| CGCCCGGCCCATGAAATACTTCTTACCTGGCGGACAGCCTAATAGCCTAGCTGTCTAACC | |
| CATGGCTGGGGGTCCTTCACACTTGTTTATACTGGCAGACGTCCCTGTGACTCTTGTCTG | |
| ATCCATGTCCAAGTTTATGCCTGTCTGACCATTGCTCTGGCGCTGGGAGCCAGACTGTGT | |
| TCCCAGCAACCCAGGGAAAACCAGGCCTGGGCTGGGCCTGGGTTCCTGAGATGGAAGGTG | |
| CAAATTCAGTACACCACCTCAATGCAAAACAAGTTCAAAGGCTTATTACTTACAGATCCT | |
| GAGCAGGGAAGGTGCAATGAGTAGGGAGGGTCATCCTCCATCCTGGGCTACATGAAGCGG | |
| GAATGAAGAGTCAGGCAAAAAGAAAGTGAGAGCTTGTGGCAATGAGAAGTATATTATGTA | |
| AGGGACTAGGGTGTGGGTCAGGTTAAGTTTGAGGGCAAATGCTTGAATGATCCCTTTAAA | |
| GGAATGGGTGGGAAGTGGGGAGCCCAGTTTGCCGGGAGGGAGAGATGCCTCGAAGTTCTT | |
| ATCTCTGGCCACTGGCTTGGACCATCTGAGTGTGGCATCTACTTCTAATGCCTAGGCAGC | |
| AACCTTTGCTGTGTCATCTCCCTTACACAAGGTTGGAAGCAAGGAGACCGGTCAGGAAGC | |
| CTTTGGTGTAACCCATGTTATTGTAATATTCATTCATTTACTCAACAGATGTTTATTGTG | |
| CACCTACTATGTGCTGAGGCCATGGCAGGCAGGCTCTGGGGATGTGGCTGAGAACAGGAC | |
| AGAGCCCCTGGTCCTTGATATCCTCAAGGATGCTCCCTCCTGGAGGCCATTAGGTTCCTG | |
| TTCCATGGTGTTCTGCTGGAACCCTCCGGTCCCAGAGTGTGCAGGAGCCTCCCCTCCTGG | |
| CAAAGGGTCTTCTCTCATGGCACAAGGGCTGCAGTACAGCCAGTCAGTGGCTCCTGGTTC | |
| CTCAAACTCAGTGAGCACTTGCCTGCCCTTCGTGCTGCCCCTCAGCTTGGGATGGCCTGA | |
| GTCAAGACCAGCCAGGAGCTCCAGGCTTCATGACCCCTTTCTTTCCCCCAGGGAGGCCCC | |
| GTCTGGCCCCTCCCCAGAATGTGACGCTGCTCTCCCAGAACTTCAGCGTGTACCTGACAT | |
| GGCTCCCAGGGCTTGGCAACCCCCAGGATGTGACCTATTTTGTGGCCTATCAGAGGTAGA | |
| GGGGACTCTCTCGGCTGGTGGATGGGAAGACTGAGGGGGTGGGTGGGGGCTGGAGGGGCT | |
| TCTCTGGGACAGCTGCACCCAGTGTGGGCAGCACTGGCTAGCTCTCTGGGCCCTACGGGA | |
| GATGGCATGTGGCCGGCATTTGGAGAGGGGCTTTTGATAAAGGTCTGGAGGTGGGGAAGA | |
| TGTTGAATGAAGAGCAGTGTACAGGTGACCAGTCTGCCGGGGCGGGGGTAAGTCTTTGAG | |
| GAAAGTTGGTGTGGGGCATGGATGTAGCTGTGGGGGCCAGAGGATGAAATTCTCAAGTGG | |
| CTGGATGAGGTGCTTGGAGCTGTCCCAGCTGATCAGTGAGGCAACTAGGTACACGGCAGA | |
| GGAGCTGTTACCTGGGCAATTAGGCATCCCTCAATGATCACACTTTTTTTCTCTTTTTTT | |
| TTTTTTTTTGAGACAGAGTCTTGGTCTGTCACCCAAGCTGGAGTGCAGTGGCTTGATCTC | |
| GGCTCACTGCAACCTCCACCTCCTGGGTTCAAGTGATTCTCCTGCCTCAGCCTCCAGAGT | |
| AGCTGGGATTACAGGCATATGCCACCACATCTGGCTAATTTTTGTATTTTTAATACAGAC | |
| GAGGTTTCTCCATGTTGCCCACGCTGGTCTCGAACTCCTGAGCTCAGGTGATCCACCCAC | |
| CTCAGCCTCCCAAAGTGTTGGGATTACAGGCGTAAGCCACCGCGCTTGGCCAAATGGTCA | |
| CACTTTTCCCGATGGGATCATTCTCAATTTGGAAGCCCAGGCAGCCACAGCGAATCCAGA | |
| GAAATCTGACAATGGAAGCAGATCCACCATCTTCGAACATAGATGGGAATCGTTCAGAGT | |
| TCTTTAGCAGGACAGTGAGATGATAGAAGCAGAAGCTCGGGAGGATTCACCTGGAGTTGG | |
| TGAGGAGGGGAAAGCAGGAAGAGGAGGGGACCACCGTGTCCTCAGGACCCGTCCTGTGCC | |
| AGGCCAAGTGCTAAGGGCCCTACGTGAATATTTCACTTCCTTCTCCCAATGTGACCAGGC | |
| AGGCTCTGTGTTTTCCCCATTCTAGAGGTGAGGGGATTGAGCTCAGAGGGTGCTGTGTCT | |
| TGTCTGAGGAAGGACGTCATGGAGCCAGAAGGGGAACTCGGGTCCGACTCCAACATTTGT | |
| GCCCTTCCTGTTGCATCACGTCATCCTTCCATGTGTGGAATCCACATGTGAGTGATGGGA | |
| GCCTGGCTTGAGCAGGGACAGACTGCAAGAGAGCTTTCAAAAGCAAGAGCGTTATCAGGT | |
| GCCAGAAAACACCTAATATTTACTGTGTGGCTGGCACTGTGTCAACACATGTAATGAACT | |
| TAATCTCACAGCAGCTCTCTGAGGACAAGTTCAGTACQCCTCTTTACAGAGGAGGAGACT | |
| GAAGCACCAAGGGTGCATGTTGCTCAAAGTCACACAGCTGGGCGTAGTATGGCTGGAATA | |
| AATTTATTAAGGAGTTGAAAGTCTATCCTCTAGGACCAAGCATGGTGGCTTACATCTGTA | |
| ATCCCAGCACTTTGGGAGGCCGAGGTGGGTGGGGAGATTGCTTGAGTCCAAGAGTTCGAG | |
| ACCAGCCTGGGTAACATGGTGAAACCCTGTCTCTACAAAAAAAAAAAATACAAAAAATTA | |
| GTGAAGTGTAGTAGCATGTGCCTGTGTTCCCAGCTACTTGGGAGGCTGAGGTGGGGAGGA | |
| TCACTTGAGCCCAGGAGATGGAGGTTGCAGTGAGCTGAGATCACACCACTGCACTCCAAC | |
| CTGAACAACAGAGCAAGATCCTAAAAAGAAAGAAAATCTATCCTCTGAACTTCTATGATA | |
| TTTTTCATGTCTTTTATACATTAGAATGGTGATATTCTAATTATATAATTTTTTTCATTT | |
| GTTAGTTGGAATTATTTTATAAAGAGATGTATCCTCTCATCTGGTATTTGATATCCAGTC | |
| ATACTATTCAAATAGGCAAGAGAGGATAAATGCTTAATTTTTTTCCTTTATCAATTTTCA | |
| AGATAATGAATTGGTTCCTTATCATCTCCCAAAGGTGATTGCTAGTTTATTATTATCATT | |
| ATGAACTCAGGCATTTAAACACATTTGGTGGTTTCAGTCTATTGCGACGTACTCTGCTCA | |
| TTGAAGCTTGAATTGCCTCATCTCTGTCCAGTGGGAGTCTCATCAAGTTTGCTCCTGAGT | |
| CCTTTTAACTTGACCCTAGTGGTCAAGTTAAATCTTTCCAGATTTAACAGATACCTTTCC | |
| AGCTGTCCATTACGACAAGATGTTCCAGGTCCCTCTGGTACAATTCCTGACCTAAAACCT | |
| GCAGTCAGCCATTTCTCCATTTAGTAAGAAATGGTTATAAAGACTATAATCTGCATGCTA | |
| GCTATGCTGATCACTACTTAGCTATTGCTTTTGGTGTTTTCAGTGAACAGAGTGATGTGT | |
| GTATACCACATAGACACACACATGTACATACTTTTTTTTTTTAGACAGAGCTTCACTCTG | |
| TCACCCAGGCCAGAGTGCAGTGGCATGATCTCGGCTCACTGCAACCTCCACCTCCTGGGT | |
| TCAAGAGATTATCCTGCCTCAGCCTACTAAGTAGTTGGGATTACAGGCGCCCACCACCAT | |
| ACCCGGCTAATTTTTGTATTTTTAGTAGAGACGGGGTTTCACCATGTTGGCCAGGCTGGT | |
| GTCGAACTCCTGACCTCAAGTGATCTGCCCCCCTCGGCCTCCCAAAATGCTGGGATTACA | |
| GGCATGAGCCATCGCACCCAGCCTACATGTACATAATTTTTAAGATAAAATGCCTAATGA | |
| GTTATACGGGTGCTTCCCATCTAAATTTAGTTCCTTAGGATTTTTACCTGACTTCTATGG | |
| TACATCTATATTTTCTTTCTTTCACACTGAGAATCCTGTTTCTCAAGGACAGGGGACATG | |
| ATAGAACTAGAATGACCCATAATTACTCATTTTCTTTATCCCAAAACATACATACTTGCC | |
| TCTTAATAGTTTCTTGCTCTTTTCGCCCAAAGGGTTTGTGATGGTCAATATTAGGTGTCA | |
| ACTTAATTGGGTTGAAGGATGCCTAGATGGCTGTTAAAGTTTTGTTTCTGGGGGTGTCTG | |
| TGAGGGTGTTGCCAGAGGAGACTGACATTTGAGTCAGTGGACTGGGAATGGAAGACTCGT | |
| CCTCACTCAGTGTGGGTGGGCACAACCCAACTGGCTGCCAGGCTGGCTGGAAAGCAGGTG | |
| GCAGATGGTGGGATAGCTTCGCTTGCTGGGTCTTCCAGCTTCCTTCTTTCTCCCGTGCGG | |
| GATGCTTCCTTCTGCTCCTCCTGCCCTTGAACATCACACTCCGGGTTTTTTGGCCTTTAG | |
| ACTCTTGGACTTAAGTTAGTGGTTTGCTGGGGGCTCTCGGATCTTTGGTCACAGACTGAA | |
| GGCTGCACTTTCAGCTTCCCTGGTTTTGAGGGTTTCAGATTCGGACTGAGTCACTATGGC | |
| TTCTTTCTTTCCCACCTTGCTGACGGCCTATCGTGGGACTTCGCCTTGTGATCGTGTGAG | |
| CCAATTCTCCTTAATAAACTCCCTTTCATATATACGTATAACCTATTAGTTCTGTTCCTC | |
| TGGAGAACCCTGACTAATAAAGGGTTGTTGCTTTTTCTTTAAAATCTAGTAATTTTATTT | |
| GACTGTGTGTTGGTATTGCTCATTCATTCTGAGTTGATATTTTTAGGCACTCAATATTCT | |
| CACTTAATACATGGTTCCAAGGCATTTTTATTTTAGGAAGGTTTTCTTAAATTATAGTTT | |
| TAGTATTTGTTCTATTCTCTTGTTTTGATTTTCTTCTTTAGGGACTCATATCACTTGTAT | |
| GTTGGATCTTCTTTTTCTGTGTTCAGTATTTGTCTTTTGGGCACAGAGACTCACACCTAT | |
| AATTCCAAGACTTTGTGAGGCATAGGTAGGAGGATCGCTTGAGCCCAGGAGTTTGAGACC | |
| AGCCTGGGCAACATGGTGAGGCCCTGTCTCAAATTAAAGAAAAAGGAGAGAATACTTGTC | |
| TTTTTCTTTCAAATGCCTTTTATCTGTCTGTCTATCTACTATTCTGCTCTCTAAATGAAA | |
| TAGGTTTCACTCTTGAGTTTTTAAAAAACTGTGTGCTTCCATGTGTGAGATTATTCAACA | |
| TCTTATTTGTAATCTTTCTCTTGGTTACATTTATTTTTCCTGAAACTCTAGTCTGCTTTT | |
| AGCTGACATGTTTGTAGCTAAGAGCGCACATTTCTTATCATAGCTTGCCGTGCTGAATTA | |
| ATTCCAATTTTCTTTTAAAACCAACATTATTGAGTTAAAATGTATATAGAATAAACTGTT | |
| CCCATTTTAAAGTATACAATTTGATGAGTTTTGACAAAAGTGGGCACCCACGTACCCACC | |
| ACCACAATCAAGATGTAAGACGTTCTCTATCACCCCAGAAAGTTCCCTCATCCACTTTGC | |
| ATTCAGGCCTCCAGATCTAGGCAACCACAGATCTGCTTTCTGACACTGTGGATTAAACTT | |
| TGCCTGTTCCAGAATTTCATATAAATGGATGTGTATAGTATGTACCCTTTCGTGTCTGGC | |
| TCCTTTCCCTCAGCATAATGTTTCTGAAATTCACCCACATTGTTACATGTATCAGTAGTT | |
| AATTCCTTTTTATTGCTGAGTAGTAATGCCATTGTATGACTATGTATGACATTTGTTAAT | |
| CCATTTTCCCGTCAGTGGATATTTGGGTTGCTTCCAGTTCTGGGCAGGTATTCATTTGCT | |
| AGGGCTGCCATATGCTTGCCCTCTGGCCTCCCAAAATTTGTGTCCTTTTCATATGCAAAA | |
| TACATTCACCCCCTCCCAACAGCCCCAAAACTCTCTTTTTTTTTTTTTTTTGAAACAGAG | |
| TTTTGCTCTTGTTGCCCAAGCTGGAGTGCAATGGTGTGATCTCGGCTCACTGCAACCTCT | |
| GCCTCCCGGGTTCAAGAGATTCTCCTGCCTCAGCCTCCTGAGTAGCTGGGATTACAGGCA | |
| TGCGCCACCACGCCTGGCTAATTTTTTATATTTTTAGTAGAAATGGGGTTTCACCGTGTT | |
| AGCCAGGCTGGTCTTGAACTCCTGACCTCAGGTGATCCGCCTGCCTTGGCCTCCCAAAGG | |
| GCTGGGATTACAGGCATGAGCTACTGCACCTGGCTAGCCCCAAAACTCTTAACCCATTTC | |
| AGCATCTACTCTAAGTCCAAAGTCTCATCTAAATCAGGTATGGGTGTGACTGGAGGTGTT | |
| ACTCATCCTGAGGCCAAATTCCTCTCCACTTATGAACCTGTGAAACCAGACAGGTTATGT | |
| GCTTTGAAAATAAAGTGATGGGACATGCATGGGATAGACTTTCCCATTCCAAAAGAGAAA | |
| AATAGGAAAGAAGGAAAGAGTGACAGGTCCCAAGCAAGTCTAAAACCTCGCAGGGCAAAT | |
| TCCATTAGATTTTAAGTTTCAAGAATAGCCCTCTTTGGCTCAGTGCTCTGCCCTTTGGGC | |
| CCACTGGGGCGGCAGCCCTATCCCCTTTGCCCTGGGTGGTGACCCTACCCTCGAGTCACT | |
| GGTTAGCAGCAGCCTAGCCTGCTGAAACTAAGGAGGGGACAGTGTTGCCTCCAGGTCTTT | |
| GGTGGCAGTGACAACCCTGCTGATCTCTGAATCATCTTCCAGGAAATTTTTCCCTATACT | |
| TGAAGGATATTGCGTGTTCACAGCCAAATAGCTCCAGCTCTTGTCCCTTTCTTTAGAATC | |
| CCAGAAGTCCAACAGCCTTCCTTCATTCTGTCCCATCTCTGTCCCCTTTAGTCAAAGCTG | |
| GAAGTGCCTCTGCTGGTATAATCCCATCAGTATGTCTAATTTCTGCTTAAATGGCTGATT | |
| AAGTCTATGAGTTGCACCTCTGATCTCTTTATCAAAAGGTTGTTCTAGCCACAACCTTAG | |
| TGTCCTCCCCAGAACATGCTTTCTCATTTTTTTTTTTGCAATGTGGATAGGCTGAAAATT | |
| TTCCAAAGCTTCAAGTTCTAGTTCCTTTTGGCTTACCAATTCTTTTCATATATCTCTTCT | |
| CTCACATTTTACTATAAGCAGTAAGAAGAAACCAGGTTGTACCTTCAGCACTTTGCTTAG | |
| AAATCTCTTCTGCTAAGCATCCAAGTTTATGTCTTTTAAATTATCTTTTTGTTATTTATT | |
| TTATATTATCATTTTTGAGATGGCTAGCCAATGATCTTTTAACTTCTAATTTCTGCAAAA | |
| CACTAGAAGACAATTCAACCAGTTCTTTGCCACTTTATAACAAGGATCACCTTTCCTCCA | |
| GTTTCCAATAACACATTCCTCTTTTCCACCTGAGACCTCACCAGAATCACCTTTAATGTC | |
| TATATTCCTACCAATAGTCTTTTTAAGGCAATATAGGCTTTCTCTAACATGCACTTCAAA | |
| CTTCAAGATTCTACCCATTATGCAATTCCAAAGCCACTTCCACATTTTTAGGTATTGATT | |
| ACCTCAGCACCTCATTTCTGGTGCCCAAATCTGCACTGGTTTGCTAGGGCTGCCATAACA | |
| AAGTACGACAGTCTGGGTAAACAACAGAATTTTATTTTCTCAAAATTCTGGAGGTTGGAA | |
| GTCCAAGGTCAAGGCGTTGCTAGGTTTAGTTTCTCCTGAAGCCTCTCTCCTTGGCTAGCA | |
| GATGGCTGCCTTCTTGCTGTGTCCTCACGTGGCTTTTTCTCTGTGTGTGTTCACTCTGGT | |
| ATCTCTTCCTCTTCTTACAAGTACACCAGTCCTACTGGATTAGGGCCCCAGCCTTATTAC | |
| TTCATTTAACCATAATTACCTCTTTAAAGCTCTTATCTCAAAACACAATACCACTGGGGA | |
| TGAGGTCTTCAACATATGAATTTTGGGGGAACTCAATTCGTCCATAATAGGGCTATTATG | |
| AATTAAGCTGCTGTGAACATTCATGTACAAGTCTTTGTGTGGATATGTTTTCATTTCTCT | |
| TAGATAAAGATCTAGGAGTATCAGCCTGGGCAACATAGTGAGACCCCATCTTTACAAAAA | |
| ATTTTCAAAATTAGCCAGGCATGGTGGCGTACACCTGTAGCCCTGCCATCTCAGGAGGCT | |
| GAGGTGGGAGGATCCCTTGAGCCCAGGGGTTTTAGACTGCAGTGAACTATGATTGCACCA | |
| CTGCACCCCAGCCTGGGTGACAGAGTGAGACTCTGTCTCTAAAAAAAAGAGAGAGAGGGG | |
| AGGAAGGAAAGAAGAAAGAGAGGGAGGGAAGGAGGGAGGGAGGGAGGGAGAAGAAAAATG | |
| GATCTAGGGTTAAGATTTAGGAGATTAGGTAATGAATGTGTACTATTACAGGGAACTGTC | |
| GAGCTGTTTCCAAAGTGACTGTACCATTGTTCATTGCCACCAACAATACATGAGAGTTCT | |
| AGTTACTCCATGTGCTTGTTACACTTAGTATTATCAGTCTTTTTCATTTTAACCATTCTA | |
| GTGAGTATGTAGTAGTATTTTATTATGGCTTTAATTTACAACTCCCTAATGATGAATGAT | |
| GTTGAACATCTTTTCATGTGCTTATTGGCCATTCATATATCTTTTGTGAAGTGACTATTC | |
| AAATATTTTTCCACTTTTTATTAGGTCATTTATTTTCTTATTATTGAGTTATCTATGAAT | |
| ACAAATCCTTTATCAGTGTATGTATTGTGATTTTTTTCCCCAGTGGCTGGCCTTTTCATT | |
| TTCGTTAGGCTTTTTTGGTGGGTTTTTTTTTTTTTTTTTGGAAGAGAAAAATATTTTAAT | |
| TTGATAAAATCCAGTATATCAGGTGTTATAGACTGAATTATACTCTACCCCACAAATTCA | |
| TATGTTGAAGCCCTAACCTCTAAGTGACTATTTGGAGATGAGCCTTTAAGGAGGTAATTA | |
| AAGTAAAATGAGATCATAAGGGTGGGCCCTAATCTAATAGGACTGGTGTCTTTATAAGAA | |
| GAGGAAGACACCAAGAGCGCATGCACACAGAAGAACGGCCTTGTGAGGACACAGCAAGAT | |
| GACGGCCATCTGCAAGCCAAGGAGAGAGGCCTCAGTAGAAACCAAACCTGCTGATGCCTT | |
| GATCTTGGACTTCCAGCCTCCAGATTTCTGTTGCTGAAGCCACCCTGCCTGTGGTGTCTT | |
| ACCATGGCAGCCCTCACAGACTAATATATCAGATTTTTTTCCTTCAACAGTTAACGCTTT | |
| TGGTGTCCTAAGCAATATTCGCCTGACCCAGGGTCATGAAGATTTTTCTTCTATGCTTTC | |
| TTCTGGAAGTTCTATAATTTTAGCTTTTACATATTTTTTTAACTTTCCTTCTTCTTGCCT | |
| TCTGTTTCTTTTAAGGCATCATCTATTGTGTTAATTTGTTCTTGTATTCCTTCTGATTTA | |
| TTCTTCACTTCTGAAATGAATTTTGCTTTTTAAAAATATATATAATTCTTTTCTGTGTCT | |
| GAGTTTTTCTAATTAGGTTTTATGTGGTTTTTTCTTGTCCTGCATCACTTTTTACTGTCT | |
| TTTGCCCATTTTGAAGTATCAGGTTCCAGTTTTGATCTGTTCATGGATATGTTTTTGTGA | |
| CATGTTTCTTCTGGCTTCTTATCATTTATTGCTTAGCTTATTAATTTCTATTCTTTCTTA | |
| TTTTCTATTATAAGTATTTAAAGCTATATGTTTTCCTCTAAGTATTACTTAGCTGTCTTA | |
| TACGTTTTCATTTGTGTTATTTGGTGATCATTCACTTTCAGCTATTTATTAATTTCCATT | |
| ATAATTCTTTCATCTATGGGTTGTTTTAAAAAATATTTTTAAGGCCAGGTGTGGTGACTC | |
| ACATCTGTAATCACAGCACTTAGGGAGGCTGAGGTGGGAGGATTGCTTGAGGCCAGAAGT | |
| TTGAGACCGGCCTAGGCAACAAAGTGAGACCCCCTCTCTACAGAATATTTTTTTAAAATT | |
| AGCTGGGCCAGGCGTGGTGGCTCATCCCAGCACCTGTAATACCAGCACTTTGGGAGGCCA | |
| AGGCAGATGGATCACCTGAGGTCAGGAGTTCGAGACCACCCTGGGCAACATGGTAAAACC | |
| CCATCTCTACTAAAATATAAAAATTAGCCAGGTGTGGTGATAGGTGCCTGTAATCCCAGC | |
| TACTTGGGAGGCTGAGGCAGGAGAATTCTTTGAACCCAGGAGGAGGAGTTTGCAGTGAGC | |
| CGAGATTGCACCACTGCACTCCAGCCTGGATGACAGAGCGAGACTCTGTCTCAAAAAAAA | |
| AAAGAAAAGAAAATTAGCTGGGTGTAGTGGCAGGTACCTGTGGTCCCAGTGACTCAGAGA | |
| CTGAGGCAGGAGGATCACCTGAGCCCAGGAGTAGAGGCTGCAGTGAGCTATGTTTGTGCC | |
| ACTGCACTCCAGCCTGTGCAACAGAGCAAGACGCTGTCTCAAAAAATATATATTTTTTTA | |
| AATTTTCAAACTTCCTTTAGTTCTCTTTTTGTTATTAACTTTTAACTGAATGTTTTGCAA | |
| TCAGAAGAAATACTTTATGAGATACCTATTCTTTAAAATTTCTTAAGAATTGCTTTGTGT | |
| TAATATTTTGTTAATAGTTCACATGTGGTTCAACCAATTTGTTTAGTTAGTTCTGTATAT | |
| GTTCATTAGACCAACTTGATAACTGTGTTGTTCTTTATTTATTTATGTATTTATTTTTCT | |
| TTGTCTATTCATCAATTGCTGGGTGAGATGTATTAAAATTTCTTGTTGTAAGTGTGGCTG | |
| TTCACTTTCTACCTGTAGTTTGTCTGTTTGCTTTATAGAGGGTGAAGTTGTTTAGTAGGC | |
| ACACATAAGTTAGAATTTTTCTGTCTTCCTGGTGAATGGAATCATTTATCATTATCTAAT | |
| GTTCTTTTCATCTTTAGTATTGCTTTGGACTTGGAAGTCTGTATTTTGTCTCCTGTTAAT | |
| ATAACTACACTGGTTCCTTTGGTGTGAATATTTGCATAGTATAACATTTTCCATGAAGAA | |
| ACAAAACAGAGGAATTGGTTCTTTCTCAAAATCTGATCTTTGTGTCAGCCCCCATCTCAG | |
| CCTTCTCCATTCATCCTTGGTCACTCCCCAAACCCAGGAGCAATCCTTGATTCTCCTTTT | |
| CCCCACATTCTACATCCAATCCGTTAGCAAGTTCTATTAGTTCTATTATTACCTCCAAAA | |
| TAGATATTGAATCCAGCCCTTTCTCACTGTCTCCACCATCATCCTGTCTCACATCCCTAC | |
| CATGGCCTCCTTGCTGGTTGACCAGAGTGATCTTGTAAAAACATGTTAGGCCAGGCACGG | |
| TGGCTCCTGCCTGTAATCCCAACACTTTGGGAGGCCAAGCGGGTGGGTCACCTGAGGTCA | |
| GGAGTTGGAGACCAGCCTGGCCGACATGGTGAAACCCTGTCTCTACTAAAAATACAAAAT | |
| TAGCCAGGTGTGGTTACGCTGGCCTGTAATCCCATCTACTCGGGAGGCTGAGGCAGGAGA | |
| ATCACTTGAACCCAGGAGGCGGAGGTTGCAGTGAGCCAAGATCATGCCACTGCACCCCAG | |
| CCTGGGCAACAGAACAAGACTCCATCTCAAAAAATAAAAATTAAAATAAAATGTTAGGCT | |
| CCCTGGGTCTCTGGCTTAGTCCATTTGTACTGCTTTAACAAAATACCTTAGAATGGTGTA | |
| ATTCTAATAATTGCTATTAATAAATAATAGCAATTAATAAATAATAGCAATTTCCTTCTC | |
| ACAGTTCTAGAGGCTGGGAAGTTCAGGGTCAAGGTGGCACCTGACTCCGTTCTGGTAAGG | |
| GCGGCTCTCTGCTTCCAAGATGGTGCCTTCTCGCTGCGTCTTCGCATAGCGGAAGGGCAA | |
| ACACTGTGTCCTCACGTGGCAGAAGAGATAGAAGGGCCAGGCAGCTCTCTGAAGTATCCA | |
| GGTTGGAGTCATGGACCTGCATGTTCCCCTCTGACATCCACAGAGTACCTATCATGGTCC | |
| TTGGCATGCAGCAGGTGGCCCATAAACGCCTGAATGAACAAACATATAGTAATGGTCGCT | |
| AGTACTAGGAATAGCAGCCACCGCAACAGTCCTGTGAGGGAGGCATTACAGATGAGGAAA | |
| CTGAGGTTTAGGGGCAAGGACCTGCCCATGGTCCCAAAGCTAGGGAGGGACAGGGCTGGG | |
| ATTCCCACTCCCATCCATCTGGCTCCAGAACCTGAGCTCCTGACCAGGCTGTTCTTATCC | |
| TGTCTCAGCCAGTGGCTGCCTGTCTGGACGGATGGACCTAAAGTCAGTCCAGCCAAACAG | |
| AGGGAAGCATGATCAACTGTTCTCTAAGTTCCCTGACCCGGAGAGGCTGAGTCCATGGCC | |
| CAAGCTCTCCTCTCTCCTCCCCCAGCTCTCCCACCCGTAGACGGTGGCGCGAAGTGGAAG | |
| AGTGTGCGGGAACCAAGGAGCTGCTATGTTCTATGATGTGCCTGAAGAAACAGGACCTGT | |
| ACAACAAGTTCAAGGGACGCGTGCGGACGGTTTCTCCCAGCTCCAAGTCCCCCTGGGTGG | |
| AGTCCGAATACCTGGATTACCTTTTTGAAGGTAGGTCTGTGGGTAAGGGACTGAGTGGAA | |
| GGCTGTCCATCCCATCGGGGAGCTGTGCTCAGTGCTCAGTGGTTCTGTTCTCCTGACCAT | |
| CTGTCTCCCACTTCCCCAAAGCAGAGGGCAGCTCCCTGQGCCAGGCCCTTTGAGATGGGG | |
| TGTGGGACCAGCAACAGCGAGGGACCATGTCTGGCAGCCTGTCAGGGAGTTAGGGGAGCT | |
| CCAGCCAGCACCAGCAATCTCACGTGCACCCTCTGCTAACAATGTTCATTATTTTCAGTT | |
| GAGCACCATTTTGGTCATGGACTACACAAGGCACTTTATATGCTTATTCCTATTTTTTTA | |
| TGTTCAGCTTCTCTCCTTAAAAACAATGTTTAAAACCAATTCTGGGCCAGGCGTGGTGGC | |
| TCACGCCTGTAATCCCAGCACTTTGGGAGGCCAAGGCAGGTGGATCACCTGAGGTCAGGA | |
| GTTTGAGACCACCCTGGCCAACATGGCAAAACCCCGTCTTTACTAAAAATACAAAAATTA | |
| GCCAGGCTTGGTGGCAGGCACCTGTAATCCCAGCTACTCGGGAGGCTGAGGCAGGAGAAT | |
| CGCTTGAACCCAGGAGGCGGAGGTTGCAGTGAGCCAAGATCACGCCCCTGCACTCCAGCC | |
| TGGGCGACAGAGCGTCTCAAAAGAAAAAAATTAATAAACAAAGAAAAAAAAACAAATTCT | |
| GTTTGCAAAAGTATTTTCTATACACTGTAGAAATTTGTGGGGTGTGGGGGGGTAAAGATG | |
| ATAGAAAAAAAAATGTCCCATGCTTACTGGCAGAAATCATGTATTGACATTGGGTGAGGA | |
| GGGCACTTTTTTTTTTTCAGTCTATTTTTAATCTTCACAGCAAACTTGTGAGGTTCATTT | |
| CCATCAACCTGAGACTCACAGAAGCTAAGAAACTTGATACCGCTAGTAACCAGTGGACTT | |
| GATACCGCTAGTAACCGGTGGACATAGATGTGAACTGGATCTTTCTGACCTCGGGCAGGG | |
| CCGGGTAACAAGGGGAGGATAAATGCCCAGACAGTGTCCTCAGAGAGCTGAGAGCTGTAA | |
| CTTGCTGCCCGGGCTTCTCACAGTGTTCAAGGACAAAATAAGGCTTTAAGAGAGAAGAGG | |
| GACAGACTGATTGCAGGGCAGCAGGAAGAGATGGTAGAGAAGGAAGAAGAGATGATTCGT | |
| GTGGAAAGAAGCTGGCTCGGTGGATGGATAAAAGAAGGGAAGGACAGATGGGTAAGAAGA | |
| AAGGGAGGATGGAGGGGATGGAGGAGGAAGCAATGGAAAAATGGGAAGGAAGGAGGTTGG | |
| ATGGAAGGATAGATGCCTATTAGGAAGGAAATATGTGTGGATAGAGAGATGGAGGATAGG | |
| AAGTATGTTAGTCAAGGTTCTCCAGAGAAACTGAACCAATAGGATATATACAGATACACT | |
| AAGAGGAGGCCAGCCGGGCGCGGTGGCTCAACCTTGTAATCCCAGCACTTTAGGAGGCCG | |
| AGGCGGGCGGATCACGAGGTCAGGAGATCAAGACCATCCTGGCTAACACAGTGAAACCCC | |
| GACTCTACTAAAAATACAAAAAAAAATTAGTTGGGCGTGATGATGTGCGCCTGTAGTCCC | |
| AGCTGCTGGGGAGGCTAAGGCAGGAGGATGGCGTGAACCCAGGAGGCAGAGCTTGCAGTG | |
| AGCTGAGATCGTGCCACTGCACTTCAGCCTGGGTGACAGAGCAAGACTCCGTCTCAAAAT | |
| AAATAAATAAATAAATAAAAAGAGGCCAGCCATGGTGGCTCACACCTGTAATCTGAGCAC | |
| TTTGGGAGGCCGAGGCGGATGGATCATTTGAGATCAGGAGTTCAAGACCAGCCTGGCCAA | |
| CATGGTGAAACCCTGTCTCTACTAAAAATACAAAAGTTACCCGTGTGTGGTGGCACACAC | |
| CTGTAGTCCCAGCTACTCAGGAGGCTGAGGCAGGAGAATTGCTTGAACTTGGGAAGCAGA | |
| GGTTGCAGTGAGCTGAGATCACGACACTGCACTCCAGCCTGGGTGACAGAGCAAGACTTT | |
| GTCTCAAAAAAAAAAAATTTATAATAAGAGGAGATTTATTATGGGAATTGGCTCATGCAA | |
| TCACAGACACAAAAATGTCCCCCAGCATGCAGTCATGGGCTGGACAACCAGGAAAGCTTG | |
| TGGTGTGATTCTGTCTGAGTCTGAAGGCCCAAGGCCAGGGGAGCAGTGGTGTAACCCCCA | |
| GTCCGAGGCCACAGGCCCGACAATCAGAGGGGCCACTGATATAAGTCCCAGAGTCCAAAT | |
| GCCGGAGAACAGGAAGCTCCAACGTCCAAGGACAGGAGAAGTTGATGTGCCAGCTCAGGA | |
| AGAGAGAATGTGAATGTGCCATTCCTCCTCCATTTTTTGTTCTCTTTGGGCCGTCAGTGG | |
| ATTGGATGATGCCTGCCCACACTGGTGAGGACAGATCATCACCAAATCTGCCGATTAAAA | |
| TGTTAATCTCTTCTGGAAAAATCCTCACAGATGGGCCCAGAAATAATGTTTTACTGTCTA | |
| CCTGGGTATCCCTTAGTGCAGCTAAATTGACACATAAACTTAACCATCACAGGCCAGGCA | |
| CTGTGGCTCACACCTGTAATCCCATCACTTTGGGAGGCCAAGGTGGGAAGATCCTTTGAG | |
| GATGAGGTAGGCAGATCACTTGAGCCTAGGAGTTCAAGACCAGCCTAGGCAACATAGGGA | |
| GACCTCGTCTCTACAAAAAAAAAAAAAATTTAAATTCGCTGGGTACGGTGGTGGGCACCT | |
| GTGGTCCCAGCTATCTGGGAGGCCAAGGTAGGAGGATGACTTGAGCCCAGGAGGTCAAGG | |
| CTGCAGTGAGCCATGATTGTTCCATTGAATTCCAGCCTCGGTGACAGAGCAACACCCTGT | |
| CTTAAAGAAAGAAAAAATTTAACCATCACAGAAGGCAGAAGAAAAGGCAGATGGGTGGAT | |
| GAGATGGGTGGGTAGATAGTATAGAAGAAAAGCGGGACATCCAGGCAGGGAAGGAAGGGC | |
| TGGAGCGAAGGAGAAGCAAGGAAGGAAGGAAGGAGAGACAAGAAGGAAGGATGTGTAGAA | |
| AGGTGGAAGAGAAAAGAAGAATGGATGTATGGGAAGAATGGATGAGTAGGTTAGAAGGCT | |
| CACTGGCTAGATAAAAGGTGAGAAGTATAAATGAATAATAAGAAAGGAGGCATAGGAAGA | |
| AAAAAATATTGGTTAGAAAGGATGATTGAGAAGAAAGGGTGGTTGGGAAGGAAGGAAGGA | |
| AGGATGGATGGATGGATGGATGGATGGGAAGGAAAGGAAGGATAAGAAGGCAGACAGGAA | |
| GGCTCTCTGGCTAGAAGAATGGCAGACAAACCACAATAATTGCTGAATGGGTAGGAATAA | |
| GACATTAGAAGAATAAAGGGAAAGACACAAAGATATTTAAAATGTTTTCATTAATTTTTT | |
| GCCTCCTCCCTGAATTTCTCCTGATTCTTCAGCCCCACATCCCAAGCCAGGGTGATCCTT | |
| CCTGCCTTTACACTCCCTCCACACTTTTTCTGCTCTCATATGTGGCCGTGGTCACTTTCT | |
| TTTGGTAGTTTGCATATTTCATTTACCCCAAACTTTCAGCTCCTGAAGGTCAGGATACAA | |
| GGAGGCCTCATCTCCGCATTCCCCTCAGCTCCCTTCCTGAAGCTTGATACCTAGTCAGTA | |
| CCCAGTGGATGTTTCCTAAACATGTAAGTAATGACATCATGAAGAAGCCACATGTTTACC | |
| TTGACCACAAACACAGGGCAAAGGTGACTAGTGTGGTCAGAGATCCCTGCTGGCTGGGAA | |
| TCAGGGAAGGCTGCATGGAAGAAGTGGCATTTTAGTTAGAACTTGAAAGGTGGTGTATTT | |
| AGTTTTCTCTGGCTGCCATATTCCTTGTCACATTGCCCTCTCCATCTTCAAGCCACTGGG | |
| CAAGGCTAGAAGGCCCTCAACAGACTATCGGTAGGAATGTGGAAGTTGAAGACTCAGAGT | |
| GCAGAAAGAAACAAGTAGCATTTTAGAGAAAAGCTAAATCCCCTCCAAGAATACCTCAAT | |
| CATCGTGAAGAGCCTGTTAGTAGACGCACTAACACTCAAGGCACTGCTTCACAAGGTAAG | |
| GAACGTGTAATTGAAAACTTGAGAAAGGAAGAAACTTGTTCTGTACTGGCAGAAAGCTTA | |
| GCAGAATTGTGTCCTGCAGTCATATGGGACACAGAGCTTGTAAATGATGAATTTGAATGC | |
| TTATCCGAGAAGGTTTCCAAATAAAATGTGGAAGGCACGGCCTGGTTTCTTCCTGCCTCT | |
| TATAGTAAAATGCAAGAGGAGAGAGAGAAAATGAGGGAAGAACTTAAACAGAAAGGAACC | |
| AGGACTTGATGATTTGGGAGGTTCTCAACCTATGCAAAAAACAATAAAATTAAGAGATTG | |
| TAGCTGGGCACAGTGGCTCATGCCTGTAATCCCAGCACTTTGAGAGTCCGAGGCGAGCAG | |
| ATCACCTGAGGTCAGGAGTTTGAGACCAGCCTGGCCAATGTGGGGAAACTCCGTCTCTAC | |
| TAAAAATACAAAAATTAGCTGGGTGTGGTGGCGGGCACCTGTAATCCCAGCTACTCAGGA | |
| GGCTGAGGTGGGAGGATCACTTGAACCCAAGAGGCGGAGGTTGCAGTGAGCCAAGATCAT | |
| GCCACTGCACTCCAGCCTGGGTGGGTGACAGAGCAAGACTCCATCTCAAAAAAAAAAAAA | |
| AAAAGAGATTGCTCCCAAAAGTGTGACATAGAGAAACAGCCAAGTATGTGATTATACCAA | |
| ACTTCAGGAAGATAAAAGATCAAAGTACTCAGTCGCTCAAAAGGCTCTTTGAAGAGATTA | |
| AGATTATAACTCACAGTCCCCTTCAATCAAACCAGGGGACTTCTAGGAAGCTGAACAGCA | |
| TTGTCCCTCAGCCATATCAGCTGGAGCCAAAAGTAGAGAAGGGCTTATCTGAAAAAAGGA | |
| TCTGTGGACCTGGCTTTTATCTAATAATGCAGTGGATTCCCCCATGACATCCATAGGAGA | |
| CCCGTAAAGTTCCTGAGACGTTTACATCCACAGAAACACTGTTAGCTTGGATTAAATGGA | |
| ACACAGAGAGTATGAAATCAAAGAAGGCTGTTGGACTCTCCAGTTTCTACTGTTGAGATG | |
| CAGACTGGTAAAACTACTTAGCTGCAAACACCTGCTACCTTTAGTGAAAAGGAAGGATAT | |
| CTCAGACGGTGAAACCAGAAGCTCAAAGGGCAGTGCTAAGAGCGAAAGAGAATTCTTCCC | |
| AGGCCTTGAAACCTAATGGAGTTTTCTTGGCTGGATTTTCAAACTGCATTGGACCATGAC | |
| CTGATTGTCCCTTTCATGTCCCCATGCTTGAGCCAGATTGTCTGCAACTGTTATCCTGTG | |
| CCTGTCCCACATTTTATGTTGGGAGCAGAAAACTTTAGTTTTGCTGGCCCACAGATAGAG | |
| AGAAACTGTACCCCGAGAGTTGTACTGACTGGACTATGCCCAGAGTCTATTTGACTCTGA | |
| CTTAGATACTGTTGATTTGGGAATTTGAGTTGATGCTGTAATGAGATGAGACTTTGGGGG | |
| ACATTGGGATGGAGTGAATGGATTTTGCATTTGAAAGAGATGTGGGTTGGGTAATCCTAG | |
| CCCACACCTGTAATCCCAGCACTTTGGGAGGCCGAGGCAGGCAGATCACCTGAGGTCGGC | |
| AGTTCGAGACCAGCCTGACCACCATGGAGAAACCCCATCTCTACTAAAAATACAAAATTA | |
| GCCAAGCATGGTAGCACATGCCTATAATCCCAGCTACTCGGGAGGCTGAGGCAGTAGAAT | |
| CGCTTGAACCCGGGAGGCAGAGGTTGCGGTGAGCCGAGATCACGCCATTGCACTCCAGCC | |
| TGGGCAACAAGAGTGAACTCCATCTAAAAAAAAAAAAAAAAGAAAGAAAGAGATGTGGAT | |
| TTTGGGTGGGGGACAGAGGGAAGACCATGGTAGGCAGAATGATCCTCTAAAGGTGCTCTG | |
| CCCTAATCCCCAGAAGCTAAGAATATGTTAGATGTCAGTATTGCGTGGCAGTAGGAATCT | |
| TAATTAACGTTATAGACTGTTATGQTTTGAATGTCCCCTCTAAAACTCCTGTTGACATTT | |
| AATCATCATTGTGATTGCATTAAGAAGTGGCCCTGTTAAAAGGTGATTTAGTCCTTAACA | |
| ACGCTGCCCCCGTGAATAGATTAAGGTCAGTCTTGCGGGAGTGTGTTTATCAAGAATGGA | |
| TTGTTAAAAAGTGAGTTCTGGCCAGGGGCAGTGGCTTATGCCACTCAGCACTTTGCGGGG | |
| CCAAGACTTGAAGTCAGTTGTTTGAGACCAGCCTGGCCAACATGGTGAAAGTCTGTCTCT | |
| ACTAAAAAATACAAAAAGTGTCCGGGAGTGGTGGCGGGCGCCTGTAATCCCAGCTGCTCA | |
| GGAGGCCGAAGCAGGAGGATCGCATGAATCCGGGAGGCAGAGGTTGCAGTGAGCTGAGAT | |
| CGCCCCGTTGCACTCCAGCCTGGGTGATAGAGCAAGACTCTGTCTCAAAAAAAAAAAAAA | |
| AAGAGGAAAGAAAGAAGAAAGAAAGAGAAAGAAAGAAAAGAAAGAAAAGGAAGGAAGGAA | |
| GGAAGGAAGGAAGGAAGGAAGGAAGGAAAGAAAGAAAGAAAGAAAGAAAGAAAGAAAGAA | |
| AGAAAGAAAGAAAGAAAGAAAGAAAGAAAAGAAAGAAGAAAAAAAGAAAGAAAAAAGAAA | |
| GAAAGAAAAAGAAAGAAAGAAAGAAAGAAAGAAAGAAAGAAAGAAAGAAAAGAAAAGAAA | |
| AGTGAGTTCTGCCCTCTCTTGCTGGCTTACTCTCACCCTCTCTTGCCCTTCCACCTGCCA | |
| CCATGGGATGACACAGCACAAAGGCCCTCACCAGATGCCAGTGCCATGCTCTTGGACTTC | |
| CAAGTCTCCAGAAACATGAGCCAAATACACTTCTGTTCATTATAAATTACCCAGCCTGTG | |
| ATATTCTGTAATAACAACACAAAATAGACTGAGACATAGATCTTCAAATAGTGAGGTTAT | |
| CCTGGATAATCCAGATGGGCCCAATCTAATCCCATGAGCCTTTAAAACTTTCTCCAGATG | |
| GAGGCAGAAGAGAAGTGGCAGAAGGGGAAGTCAGAGAGATTTGAAGCATAAACAGGACTC | |
| CATGGTGCCGTTTCTGGTTTGACGATGGAGTGGTAACGTGATGAAAAATGTGGGTGCCTT | |
| CCGGAGCTGAGAGGCTCCCACTAACAATCGGCCAGGAAACAGGGACCACAGCCCTACAGC | |
| CACAAAGAACTAAGTTTTGCTGACAACCCAAGGGGGCTTGGAAGTGTCTTCTCCCCCATC | |
| GGTTCCAGATGTGAGACCCAGAGCGAAGGAACCAGCTGAGCCCACCTGGACTTCTGACCT | |
| AGAGAACTGTGAGATAATAAGTTTGTATCATTTTTAAGGCACTGTGTGTGTGGTAATTTG | |
| TTATGACAGCAATAGAAAATGAATCCAGATGGGCAGGATCTGCCAGGCCAGTGACATGTG | |
| GAGGGCACCCAGGCGGATGGGATGGCATGAGAGAAGGCAGGTCAGCAATGAGCTTGCCCA | |
| GGTCACCTCTCCTCTCTAAGCCTCAGTTTTCCTCTCTATGAAATGAGAGTAGTGATATCT | |
| CCCTCCCAGGGTCAGTGCAAGGCTGAAATAACAGATTATAAGGTGCTAGGTGCACAAGAA | |
| GTGTTTGAAACATGCTAGTTGCTTTTCCATTTCCAAGAGAGCTCTCTGGTCTTGGGGGAT | |
| GGAGGCAGTGCGGCCCCTCGGGATTACTGACAGGTCCTGCTCTGTTTCTGCAGTGGAGCC | |
| GGCCCCACCTGTCCTGGTGCTCACCCAGACGGAGGAGATCCTGAGTGCCAATGCCACGTA | |
| CCAGCTGCCCCCCTGCATGCCCCCACTGGATCTGAAGTATGAGGTGGCATTCTGGAAGGA | |
| GGGGGCCGGAAACAAGGTGGGAAGCTCCTTTCCTGCCCCCAGGCTAGGCCCGCTCCTCCA | |
| CCCCTTCTTACTCAGGTTCTTCTCACCCTCCCAGCCTGCTCCTGCACCCCTCCTCCAGGA | |
| AGTCTTCCCTGTACACTCCTGACTTCTGGCAGTCAGCCCTAATAAAATCTGATCAAAGTA | |
| TGATGACCTACAGGAGGCCTGCTTGCCAAGTCAACAGATTCAGTACAGAAAAACTGAAAA | |
| ATACAGATAAGCTCTAAGAAGCAGACCAAAAGTACCCAGAGATGACCGCACATCACTCTG | |
| GTGTATATCCAATTTCAGATTTGTTTTCTGTGTATGCATGTGTGTATAGCTGCATTTATT | |
| TATGGCAAGGGCTGGCAGACTTTCCCGAAGAAGGCCAGATAGTCGATATGTTTGGCTTCA | |
| TGGGCCGTATGTTCGCTCAGGACTACTCAACGCTGCAGTTATAGCACAAAAGGAGCCGTA | |
| GCCTATACGTAAATGAATGGGCATCGCTGGGTTCCAGTAAAACTGTTTACAGGCCAGGTG | |
| CGGTGGCTCATGCCTGTAATCTCAGTACTTTGGGAGGCCGAGGTGGTGGGAGGATTACCT | |
| TAGCCCAGGAGTTCAAGACCAGCCTGGGGAACATGGTGAAACATTATCCCTACAAAAAAA | |
| AAAAAAGCTGGGTGTGGTGATGCATGCTTGTGGTCCCAGCTGCTTGGGATGCTGAGGCAG | |
| GAGGATCGCTCGAGCCCAGGAAGCAAGGCCACAGTGAGCCATGATCGCACCACTGCACTT | |
| TAGTCTGGGCAACAGAGTGAGACCTTGTCTCAAAAAAAACAAAAAATAAAACTTTTTACA | |
| TAAACAAGTGGCCAACCAGACTTGGTCCCTGGGCCTCTGCTCTTGAATGTTCTTGCTTCC | |
| ACTAAAGTAACATTCACACTCCCGATTTTTGCATACTCTGGGTTCTGGGGAATATAGATC | |
| CGAATCCAGCGTGGTTCCTGCCTTCAAGAACCTCACAAATATTCTAGACCAGCACTGCCC | |
| AATAGAAAGAAATATAATGCAAGCCACATGTGCAGTTTTAAGTGTTCCATGTTAAATTAA | |
| GTAAAAAGAGACGGGTAAATCGAATTTTAATAACAGATTTTACTTCATCCAATTGAATGG | |
| TATCATTTCAATGAGCAATTCTGATAGTGATTGAGATCTTTTACATTCTTTTTCACTACG | |
| TCTTTAAAATCTGATGTGTGTTTTGTACTTGGAACACTTCTCAGTGTGGACCAGATGCAT | |
| TTCACATACTCAGTAGTCACGCGTGGCCAGTGCCTTCCATACCACACAGTGCAGCATCTG | |
| TAGAGGTTTCCTCCACTGCTGATAGACTAGGAGACCCCAAGATGGAAAGCCTGAAGAATC | |
| TGCTCCTCGAAGTAGGGACCTTAATGGGGTGCACGCCAGGGCGACCCCAAGTGGTAGGCT | |
| GCTTTTGAACCATGGCTATCCCTACCTCTAGACTCAGCTGAAAAGAACTCAGGTAGTCTT | |
| GGGAAGTGCTTCCTCAATGCTTAAACTTTAATGCAGGAAAAGAATAGAAAGTTCAGGCAA | |
| GGAGGGAGGATCACTTGAGGCTGGGAGTTCGAGACCAGCCTGGGCAACAGCAAGACCTTG | |
| CCTATACAAAAAATAATTTTAAAAAATTACCCAGGTATGGTGGTGTGGATCTGTAGTCCC | |
| TAGTTACTTGGAGAGCTGAGGTAGGAGGATCGCTTGAGCCCAGGAGTTTGAGGCTGCAGT | |
| GAGCTGTGATCACACCACTGCACTTTGGCCTGGGTGACAGAACCAAACCCTATCCCCTAC | |
| AAAAAAACAAAAAAAAAAAACAAAAAAAAACACCCTACCATGTCTGCCAACCCCACTCTG | |
| TCCTGGCTGTGTGAAACCAGTCCCCACAGCAGCTCTGCCACTCTCTGCTTCTTTTCCAAA | |
| CAGACCCTATTTCCAGTCACTCCCCATGGCCAGCCAGTCCAGATCACTCTCCAGCCAGCT | |
| GCCAGCGAACACCACTGCCTCAGTGCCAGAACCATCTACACGTTCAGTGTCCCGAAATAC | |
| AGCAAGTTCTCTAAGCCCACCTGCTTCTTGCTGGAGGTCCCAGGTGGGTATCAAGTGGTG | |
| CAGAAGGAGAAACTTTCCCTCTGGGCCTTGGGAGCTTCGTGACACAGTGGTTAAGAACAT | |
| GAGCCTAGAGATAGACTCGCCTGGATTAAAACCACACTCATTGTGTGTCTTTGGGCAGCT | |
| TACATAATGCCCCGAACCTTGGTTTGCACAGTCTGCAGGATGGGTTTATTCTTGTGAGGA | |
| TTAAATAGGGTCATGTATGTGAAGCACTCGGCACAGGTGCAGTTGTAGACAAGAGCCATT | |
| GTTGTTTCTCTCATTGTTATTTTTCCTTCCTTAGAAGCCAACTGGGCTTTCCTGGTGCTG | |
| CCATCGCTTCTGATACTGCTGTTAGTAATTGCCGCAGGGGGTGTGATCTGGAAGACCCTC | |
| ATGGGGAACCCCTGGTTTCAGCGGGCAAAGATGCCACGGGCCCTGGTATAGCAAATCTGG | |
| GGGTGTGCGGCAGGTGGGGAGGGGTTGAGAGTAAGGGAGTGGGGCTGGAGCTATGAGTTG | |
| TTCAGATAGAATATCAAGATGGTCCAGACTCTTGGACCAAAACATCTATCTTTGTGTCTG | |
| AATTTCCACCATTAGTAATGCATTCATTTAGTCCTGAATAAAATGGCAAACAGGCCCTGG | |
| AGGGAGCAGTGCCTTAAGTTCCTTTGAGATAAATAACTTCACCTCTGCTAAGGATGTGTC | |
| AGCTGCTGAGAGCAGAGCCCCTGGCCTTGGACCTCAGGAGAGACACTCAAAAGGGGAGGA | |
| GAGGAGGCACCAAAGGGGACATCTTAAAAGAGTTCCPATTTTTAGTTCACACTTTAACCC | |
| AGGATAAGCTGTGTCCTGGCTGACCTTGGAGTTTCTTCCCTGGTCTGCTGCGTCTCTCCC | |
| TTAGAACCTAGGGGCGAGCTGGGGCAGGGGAAGCCCAGGAGGTGATATAGGTCGGCCCTG | |
| TTCAGATGAGGGCTGGCAGGGGCAGCTTGGGCATATGCGAGGCTCCGATGGGCATGGGGG | |
| CTTTGAGGATGGATTCTGAGTGTCCCTGCATCGTGGCAGGGTGGCAAAGGGAGCATTTCC | |
| AAATTTCCTGGCTCCAGGATCTGTGGGAGAATCCCACTAACTGTCAGGGTGACAACCTCG | |
| GGTAGACATGTCTGTGCCCTGCCCCGTGCCCTCAGCCTTCCTGTTAAGAGCACACCAGCT | |
| GGATTTGCAACTCCCAGCGCCTGCACCCAATGGGCTTTCTCTGGCCTCTGGAGCCCACAT | |
| TGCCCCTGCATGTGGCAGGCTGCAAGTGTCACAGCCACCAGCTCTTCCATTCCTCAACAA | |
| TGACTGTGGGTAAATAGCCCAGGAGCGTCCCCCTCCTGGGATGGTTCTGAGGTGCGTGTG | |
| CCCAGTGGCTCCCTGAGTTGCCAGCAGGATTAAGTGCCAGTAGCCCTAGTGGTCAGCTGC | |
| TTGATAACACCCTGCTTCCTGGCTGCTCCCCCAGTCCCATCTGGTGTGTTCTGGGATCAT | |
| CTCCCAAAGAAACTGCTTACACTTGAAGCCTTGTCTGAGGTCTGTTTCTAGGGGAATTCA | |
| GATGACGATAATTATGCTTCAGGAAAGCCTAAATTTTCTGCTTTTCTCTCCCCTACCCAA | |
| ATCAGGACTTTTCTGGACACACACACCCTGTGGCAACCTTTCAGCCCAGCAGACCAGAGT | |
| CCGTGAATGACTTGTTCCTCTGTCCCCAAAAGGAACTGACCAGAGGGGTCAGGCCGACGC | |
| CTCGAGTCAGGGCCCCAGCCACCCAACAGACAAGATGGAAGAAGGACCTTGCAGAGGACG | |
| AAGAGGAGGAGGATGAGGAGGACACAGAAGATGGCGTCAGCTTCCAGCCCTACATTGAAC | |
| CACCTTCTTTCCTGGGGCAAGAGCACCAGGCTCCAGGGCACTCGGAGGCTGGTGGGGTGG | |
| ACTCAGGGAGGCCCAGGGCTCCTCTGGTCCCAAGCGAAGGCTCCTCTGCTTGGGATTCTT | |
| CAGACAGAAGCTGGGCCAGCACTGTGGACTCCTCCTGGGACAGGGCTGGGTCCTCTGGCT | |
| ATTTGGCTGAGAAGGGGCCAGGCCAAGGGCCGGGTGGGGATGGGCACCAAGAATCTCTCC | |
| CACCACCTGAATTCTCCAAGGACTCGGGTTTCCTGGAAGAGCTCCCAGAAGATAACCTCT | |
| CCTCCTGGGCCACCTGGGGCACCTTACCACCGGAGCCGAATCTGGTCCCTGGGGGACCCC | |
| CAGTTTCTCTTCAGACACTGACCTTCTGCTGGGAAAGCAGCCCTGAGGAGGAAGAGGAGG | |
| CGAGGGAATCAGAAATTGAGGACAGCGATGCGGGCAGCTGGGGGGCTGAGAGCACCCAGA | |
| GGACCGAGGACAGGGGCCGGACATTGGGGCATTACATGGCCAGGTGAGCTGTCCCCCGAC | |
| ATCCCACCGAATCTGATGCTGCTGCTGCCTTTGCAAGGACTACTGGGCTTCCCAAGAAAC | |
| TCAAGAGCCTCCGTACCTCCCCTGGGCGGCGGAGGGGCATTGCACTTCCGGGAAGCCCAC | |
| CTAGCGGCTGTTTGCCTGTCGGGCTGAGCAATAACATGCCCCTCCCTCCTGTGACCCGCC | |
| CTCTTTAGGCTGAGCTATAAGAGGGGTGGACACAGGGTGGGCTGAGGTCAGAGGTTGGTG | |
| GGGTGTCATCACCCCCATTGTCCCTAGGGTGACAGGCCAGGGGGAAAAATTATCCCCGGA | |
| CAACATGAAACAGGTGAGGTCAGGTCACTGCGGACATCAAGGGCGGACACCACCAAGGGG | |
| CCCTCTGGAACTTGAGACCACTGGAGGCACACCTGCTATACCTCATGCCTTTCCCAGCAG | |
| CCACTGAACTCCCCCATCCCAGGGCTCAGCCTCCTGATTCATGGGTCCCCTAGTTAGGCC | |
| CAGATAAAAATCCAGTTGGCTGAGGGTTTTGGATGGGAAGGGAAGGGTGGCTGTCCTCAA | |
| ATCCTGGTCTTTGGAGTCATGGCACTGTACGGTTTTAGTGTCAGACAGACCGGGGTTCAA | |
| ATCCCAGCTCTGCTCTTCACTGGTTGTATGATCTTGGGGAAGACATCTTCCTTCTCTGCC | |
| TCGGCTTCCTCATCTGCAGCTACGCCTGGGTGTGGTGAGGGTTCTAGGGGATCTCAGATG | |
| TGTGTAGCACGGAGCCTGCTGTGTCCTGGGTGCTCTCTACGTGGTGGCCGGTAGAATTCT | |
| CCATCTATCCAGGCTCCAGGAGACCCCTGGGCATCTCCCACCTGTGGCCCCTAAACCCAG | |
| AGTGACTGAGAGCACTTACCATTCAGCTTGTCTCATCCCCAGTCTACCTCCTTCCTTCTA | |
| CCCTCACTGCCTCCCAGTCAGGAGAGTGAGCTCTCAGAAGCCAGAGCCCCACCCAAGGGG | |
| ACCCTGGTCTCTCCGCCTTCACCTAGCAATGGGAACCCTGCTTCCCAGGGGAGGAACCAA | |
| CTGCTCCACCTTCTAGGGACCCAGTTTGTTGGAGTAGGACAGTAACATGGCAGGAATCGG | |
| ACTTCTGGGCCTGTAATCCCAGTTTGGATGGCACGTTAGACTCTTGGTTGACCGTTGTGG | |
| TCCTTAGAAGTCCCATTCTCCCTTCCAGTTATGAGAAACCAATGCCTTCTAGATTCAGGT | |
| GACTATCCTTACCTGGGGGTGCTGATGCATCCTCAGTTAACCTACACCCACCTGAATATA | |
| GATGAGCGTAGCTGAGTTTTCACCCGTAGGACCGAAGTGTTTTGTGGTGGAGTATCTGAA | |
| CAACCTTGGCTCTGTGGCCATTCAACCTGCCAGGACTAACATTTCTGGATTTGTGAAGAA | |
| GGGATCTTCAAAGCCATTGAACCCACAGAGCTGTGTTGCTTTAAAGCCACCACAAGGGTA | |
| CAGCATTAAATGGCAGAACTGGAAAAGCTTCTTAGGGCATCTCATCCAGGGATTCTCAAA | |
| CCATGTCCCCCAGAGGCCTTGGGCTGCAGTTGCAGGGGGCGCCATGGGGCTATAGGAGCC | |
| TCCCACTTTCACCAGAGCAGCCTCACTGTGCCCTGATTCACACACTGTGGCTTTCCACGT | |
| GAGGTTTTGTTTAGAGGGATCCACTACTCAAGAAAAAGTTAGCAAACCACTCCTTTTGTT | |
| GCAAAGGAGCTGAGGTCAAGGGTGGCAAAGGCACTTGTCCAAGGTCGCCCAGCAGTGCTG | |
| CTCTGATGACTTGTGCACATCCCCAAGGGTAAGAGCTTCGATCTCTGCACAGCCGGGCCA | |
| ACCTCTGACCCCTTGTCCATGTCAGTAAAATATGAAGGTCACAGCCAGGATTTCTAAGGG | |
| TCAGGAGGCCTTCACCGCTGCTGGGGCACACACACACACATGCATACACACATACGACAC | |
| ACACCTGTGTCTCCCCAGGGGTTTTCCCTGCAGTGAGGCTTGTCCAGATGATTGAGCCCA | |
| GGAGAGGAAGAACAAACAAACTACGGAGCTGGGGAGGGCTGTGGCTTGGGGCCAGCTCCC | |
| AGGGAAATTCCCAGACCTGTACCGATGTTCTCTCTGGCACCAGCCGAGCTGCTTCGTGGA | |
| GGTAACTTCAAAAAAGTAAAAGCTATCATCAGCATCATCTTAGACTTGTATGAAATAACC | |
| ACTCCGTTTCTATTCTTAAACCTTACCATTTTTGTTTTGTTTTGTTTTTTTGAGTCGGAG | |
| TTTTGTTCTTGTTGCCTAGGCTGGAGTGCAGTGGTGCGATCTCGGCTCACTGCAACCTCC | |
| ACCTCCCGGGTTCAAGTGATTCTCCTOCCTCAGCCTCCCAAGTAGCTGGGATTACAGGCA | |
| CCCGCCACCACACCTGGCTAATTTTTTTGTATTTTTAGTAGAGATGGGGTTTCACCATGT | |
| TGGCCAGGCTGGTCTCGAACTCCTGACCTCAGGTGATCCGCCCGCCTCGGCCTCCCAAAG | |
| TGCTGGGATTACAGGCGTGAGCCACCGCGCCCAGCCAAACCTTACTATTTTTTTAAAGAA | |
| TTTTTTCCAGAGTTTAATTTCTGACATAGCTTAAGTTTTCCAGTAACTCTAAACTCCATC | |
| TCCTTTATCGTCATTAAGTCATTCACAAAAAGCCAGGAGAAGCATTTGGAAAGGGCATGA | |
| TAATCAGTATAATAATT | |

Table 8 presents a correlation between the genomic sequence shown in Table 7 and the locations of the corresponding regions of the cDNA sequence shown in Table 1.

TABLE 8

| Region in Genomic | Sequence | | Corresponding Region |
|---|---|---|---|
| Sequence of Table 7 | Attribute | Length | in cDNA sequence |
| 1-2000 | 5′ sequence | 2000 | — |
| 2001-2058 | Exon #1 | 58 | 1-58 |
| 2059-8391 | Intron #1 | 6333 | — |
| 8392-8515 | Exon #2 | 124 | 59-182 |
| 8516-19645 | Intron #2 | 11130 | — |
| 19646-19830 | Exon #3 | 185 | 183-367 |
| 19831-27533 | Intron #3 | 7703 | — |
| 27534-27676 | Exon #4 | 143 | 368-510 |
| 27677-29583 | Intron #4 | 1907 | — |
| 29584-29743 | Exon #5 | 160 | 511-670 |
| 29744-30034 | Intron #5 | 291 | — |
| 30035-30165 | Exon #6 | 131 | 671-801 |
| 30166-31325 | Intron #6 | 1160 | — |
| 31326-32084 | Exon #7 | 759 | 802-1560 |
| 32085-32087 | Stop | 3 | 1561-1563 |
| 32088-34757 | 3′ sequence | 2667 | — |

Several sequence polymorphisms have been identified in the sequence shown in Table 7. These are summarized in the Table 9:

TABLE 9

| SNP | Position | SNP Changes | |
|---|---|---|---|
| variation | 32959 | allele = “C” | allele = “A” |
| variation | 31266 | allele = “C” | allele = “T” |
| variation | 30960 | allele = “T” | allele = “C” |
| variation | 29048 | allele = “C” | allele = “T” |
| variation | 28753 | allele = “G” | allele = “A” |
| variation | 23830 | allele = “G” | allele = “A” |
| variation | 8811 | allele = “C” | allele = “T” |

CRF2-like nucleic acids and polypeptides of the invention (including those shown in Table 1) are referred to herein as “CRF2-13” nucleic acids and polypeptides.

A CRF2-13 nucleic acid, and the encoded polypeptide, according to the invention are useful in a variety of applications and contexts. For example, sequence comparison reveals that the disclosed CRF2-13 nucleic acid (Table 1) encodes a Type II cytokine receptor. One or more secreted receptor chains may be associated with, and/or modulate the activity of, another membrane bound member of CRF2, or a membrane bound receptor of another family. Alternatively, or in addition, the receptor chains disclosed herein may act alone or in combination with another soluble receptor. In effect, the receptor can also be a ligand.

A soluble form of the CRF2-13 polypeptide of the invention (e.g., a polypeptide that includes amino acids 21-230, amino acids of SEQ ID NO:2) may additionally be used as a soluble receptor antagonist. Soluble receptor antagonists that block the activity of specific cytokines, e.g., TNF, are known in the art. A soluble CRF2-13 polypeptide of the invention can similarly block the activity of a cytokine that acts through a CRF2 member. Examples of such polypeptides include IL-10, IL-19, IL-20, IL-22, AK155, mda-7 or an interferon, such as interferon alpha, interferon beta, or interferon gamma. In one embodiment, a soluble CRF2-13 polypeptide of the invention is used to antagonize the function of IL-22. IL-22 is distantly related in sequence to IL-10 and is produced by activated T cells. IL-22 signaling into a cell is mediated by its receptor chains, IL-22R and CRF2-4, both members of the CRF2 family. The CRF2-4 receptor was originally reported to serve as a second component in IL-10 signaling. IL-22 has been reported to inhibit IL-4 production from human Th2 T cells and to induce acute phase proteins in the liver of mice.

CRF2-13 nucleic acids and polypeptides according to the invention may additionally be used to identify cell types that make the invention or bind to the invention in a population of cells. The CRF2-13 nucleic acids and polypeptides can also be used for immunomodulation, inflammation, immunosuppression, allergy, asthma, autoimmunity (including rheumatoid arthritis and multiple sclerosis), repair of vascular smooth muscle cell after vascular injury or disease, transplantation and cancer based on the ligand that associates with this soluble receptor, alone or in conjunction with another receptor, and the impact that this ligand has on the above mechanisms and/or pathologies.

For example, a CRF2-13 polypeptide and/or soluble form of a CRF2-13 polypeptide of the invention may exhibit one or more of the following activities: (1) modulation,-e.g., it may antagonize a signal transduction pathway mediated by a cytokine (such as IL-10 or IL-22); (2) modulation of cytokine production and/or secretion (e.g., production and/or secretion of a proinflammatory cytokine); (3) modulation of lymphokine production and/or secretion; (4) modulation of expression or activity of nuclear transcription factors (5) competition with cytokine receptors for cytokine ligands; (6) modulation of cell proliferation, development or differentiation, e.g., cytokine-stimulated (such as IL-10 or IL-22) production, development, or differentiation; (7) modulation of cellular immune responses; modulation of cytokine-meditated proinflammatory actions; and/or promotion and/or potentiation of immune reactions.

A CRF2-13 polypeptide of the invention may directly, by association with a membrane bound receptor, or indirectly, by its association with a soluble ligand affect or effect one or more of the following cell types: circulating or tissue-associated cells: T cells, B cells, NK cells, NK T cells, dendritic cells, macrophages, monocytes, neutrophils, mast cells, basophils, eosinophils, as well as cells in the respiratory tract, pancreas, kidney, liver, small and large intestine. A CRF2-13 polypeptide of the invention may additionally modulate upregulation of humoral immune responses and cell-mediated immune reactions; modulate the synthesis of proinflammatory cytokines and chemokines; and modulate inflammatory responses associated with injury, sepsis, gastrointestinal and cardiovascular disease, or inflammation following surgery.

For efficient production of the protein, it is preferable to place the CRF2-13 sequences under the control of expression control sequences optimized for expression in a desired host. For example, the sequences may include optimized transcriptional and/or translational regulatory sequences (such as altered Kozak sequences). In addition, the mature amino terminus of a CRF2-13 protein may be operably linked to a non-CRF2-13 signal sequence based on a hypothetical or empirically determined of the mature amino terminal end of the protein.

A CRF2-13 fusion protein can be used to identify and determine binding partners using assays known in the art. These assays include, e.g., either histological, immunochemical, BIACORE or cell biology based assays.

Assays can also be performed in order to determine whether a CRF2-13 protein of the invention associates with cell types that already express other members of the CRF2 family. A CRF2-13 of the invention can also be examined for its ability to modulate the activity of known or novel cytokines (e.g., by inhibiting or otherwise antagonizing the functions of a cytokine).

For example, several novel IL-10 like molecules have been cloned. IL-22 is one of these molecules. It has been reported that this molecule blocks the production of IL-4 by Th2 cells (human) and initiates an acute phase response (mice). A finding that CRF2-13 binds to and inhibits IL-22 (or other IL-10 like molecules) indicates a CRF2-13 invention can be used to treat or prevent diseases associated with high levels of the IL-22 polypeptide.

It is also contemplated that a CRF2-13 polypeptide of the invention associates with other receptors and/or their associated cytokines within the CRF2 family. For example, a CRF2-13 of the invention may associate with either chain of the IL-22R and affect the function of the receptor or the IL-22 ligand.

Also within the invention is a nucleic acid that encodes a polypeptide that includes amino acid sequences 21-520 of SEQ ID NO:2, e.g., a nucleic acids 61-1560 of SEQ ID NO:1. Examples of such nucleic acid molecules are that encode polypeptides with the amino acid sequences of SEQ ID NO:2.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes an CRF2-13 nucleic acid and a pharmaceutically acceptable carrier or diluent.

In a further aspect, the invention includes a substantially purified CRF2-13 polypeptide, e.g., any of the CRF2-13 polypeptides encoded by an CRF2-13 nucleic acid, and fragments, homologs, analogs, and derivatives thereof. The invention also includes a pharmaceutical composition that includes an CRF2-13 polypeptide and a pharmaceutically acceptable carrier or diluent.

In still a further aspect, the invention provides an antibody that binds specifically to an CRF2-13 polypeptide. The antibody can be, e.g., a monoclonal or polyclonal antibody, and fragments, homologs, analogs, and derivatives thereof. The invention also includes a pharmaceutical composition including CRF2-13 antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing an CRF2-13 polypeptide by providing a cell containing an CRF2-13 nucleic acid, e.g., a vector that includes an CRF2-13 nucleic acid, and culturing the cell under conditions sufficient to express the CRF2-13 polypeptide encoded by the nucleic acid. The expressed CRF2-13 polypeptide is then recovered from the cell. Preferably, the cell produces little or no endogenous CRF2-13 polypeptide. The cell can be, e.g., a prokaryotic cell or eukaryotic cell.

The invention is also directed to methods of identifying an CRF2-13 polypeptide or nucleic acid in a sample by contacting the sample with a compound that specifically binds to the polypeptide or nucleic acid, and detecting complex formation, if present.

The invention further provides methods of identifying a compound that modulates the activity of an CRF2-13 polypeptide by contacting an CRF2-13 polypeptide with a compound and determining whether the CRF2-13 polypeptide activity is modified.

The invention is also directed to compounds that modulate CRF2-13 polypeptide activity identified by contacting an CRF2-13 polypeptide with the compound and determining whether the compound modifies activity of the CRF2-13 polypeptide, binds to the CRF2-13 polypeptide, or binds to a nucleic acid molecule encoding an CRF2-13 polypeptide.

In another aspect, the invention provides a method of determining the presence of or predisposition of an CRF2-13-associated disorder in a subject. The method includes providing a sample from the subject and measuring the amount of CRF2-13 polypeptide in the subject sample. The amount of CRF2-13 polypeptide in the subject sample is then compared to the amount of CRF2-13 polypeptide in a control sample. An alteration in the amount of CRF2-13 polypeptide in the subject protein sample relative to the amount of CRF2-13 polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the CRF2-13 is detected using an CRF2-13 antibody.

In a further aspect, the invention provides a method of determining the presence of or predisposition of an CRF2-13-associated disorder in a subject. The method includes providing a nucleic acid sample, e.g., RNA or DNA, or both, from the subject and measuring the amount of the CRF2-13 nucleic acid in the subject nucleic acid sample. The amount of CRF2-13 nucleic acid sample in the subject nucleic acid is then compared to the amount of an CRF2-13 nucleic acid in a control sample. An alteration in the amount of CRF2-13 nucleic acid in the sample relative to the amount of CRF2-13 in the control sample indicates the subject has a tissue proliferation-associated disorder.

In a still further aspect, the invention provides a method of treating or preventing or delaying an CRF2-13-associated disorder. The method includes administering to a subject in which such treatment or prevention or delay is desired an CRF2-13 nucleic acid, an CRF2-13 polypeptide, or an CRF2-13 antibody in an amount sufficient to treat, prevent, or delay a tissue proliferation-associated disorder in the subject. Examples of such disorders include rheumatoid arthritis and multiple sclerosis.

CRF2-1 Nucleic Acids

The nucleic acids of the invention include those that encode a CRF2-13 polypeptide or protein. As used herein, the terms polypeptide and protein are interchangeable.

In some embodiments, a CRF2-13 nucleic acid encodes a mature CRF2-13 polypeptide. As used herein, a “mature” form of a polypeptide or protein described herein relates to the product of a naturally occurring polypeptide or precursor form or proprotein. An example of a CRF2-13 nucleic acid encoding a mature form of a CRF2-13 polypeptide is a nucleotide sequence encoding amino acids 21-520 of SEQ ID NO:2 (e.g., nucleotides 61-1560 of SEQ ID NO:1). The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the CRF2-13 nucleic acids of the invenation are the nucleic acid whose sequence is provided in nucleotides 1-1560 of SEQ ID NO:1, SEQ ID NO:1 itself, or a fragment of one of these sequences. Additionally, the invention includes mutant or variant nucleic acids of SEQ ID NO:1, or a fragment thereof, any of whose bases may be changed from the corresponding bases shown in SEQ ID NO:1, while still encoding a protein that maintains at least one of its CRF2-13-like activities and physiological functions (ie., modulating angiogenesis, neuronal development). The invention further includes the complement of the nucleic acid sequence of SEQ ID NO:1, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode CRF2-13 proteins or biologically active portions thereof. Also included are nucleic acid fragments sufficient for use as hybridization probes to identify CRF2-13-encoding nucleic acids (e.g., CRF2-13 mRNA) and fragments for use as polymerase chain reaction (PCR) primers for the amplification or mutation of CRF2-13 nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CRF2-13 nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, or a complement thereof, can be isolated using standard molecular biology techniques and the sequence information provided herein. Using all or a portion of the nucleic acid sequence of SEQ ID NO:1 as a hybridization probe, CRF2-13 nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to CRF2-13 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO:1, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO:1, or a portion of this nucleotide sequence. A nucleic acid molecule that is complementary to the nucleotide sequence shown in SEQ ID NO:1 is one that is sufficiently complementary to the nucleotide sequence shown in SEQ ID NO:1 that it can hydrogen bond with little or no mismatches to the nucleotide sequence shown in SEQ ID NO:1, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO:1, e g., a fragment that can be used as a probe or primer, or a fragment encoding a biologically active portion of CRF2-13. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a CRF2-13 polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a CRF2-13 polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human CRF2-13 protein. Homologous nucleic acid sequences include those nucleic acid sequences that encode conservative amino acid substitutions (see below) in SEQ ID NO:2, as well as a polypeptide having CRF2-13 activity. Biological activities of the CRF2-13 proteins are described below. A homologous amino acid sequence does not encode the amino acid sequence of a human CRF2-13 polypeptide.

The nucleotide sequence determined from the cloning of the human CRF2-13 gene allows for the generation of probes and primers designed for use in identifying and/or cloning CRF2-13 homologues in other cell types, e.g., from other tissues, as well as CRF2-13 homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:1; or an anti-sense strand nucleotide sequence of SEQ ID NO:1; or of a naturally occurring mutant of SEQ ID NO:1.

Probes based on the human CRF2-13 nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CRF2-13 protein, such as by measuring a level of a CRF2-13-encoding nucleic acid in a sample of cells from a subject e.g., detecting CRF2-13 mRNA levels or determining whether a genomic CRF2-13 gene has been mutated or deleted.

A “polypeptide having a biologically active portion of CRF2-13” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of CRF2-13” can be prepared by isolating a portion of SEQ ID NO:1 that encodes a polypeptide having a CRF2-13 biological activity (biological activities of the CRF2-13 proteins are described below), expressing the encoded portion of CRF2-13 protein (e.g., by recombinant expression in vitro) and assessing the activity of the encoded portion of CRF2-13. For example, a nucleic acid fragment encoding a biologically active portion of CRF2-13 can optionally include a cytokine-binding domain. In another embodiment, a nucleic acid fragment encoding a biologically active portion of CRF2-13 includes one or more regions.

Polymorphisms in CRF2-13 Associated Sequences

The invention also provides polymorphic forms of CRF2-13 nucleic acid sequences as well as methods of detecting polymorphic sequences in CRF2-13 sequences The polymorphic forms include genomic sequences corresponding to exons and/or introns associated with CRF2-13. The polymorphisms can be provided on various isolated CRF2-13 nucleic acids. For example, the polymorphism can be provided on an isolated polynucleotide comprising at least 10 contiguous nucleotides of SEQ ID NO:3 that include the polymorphic sequences shown in Table 6. Alternatively, the polymorphism can be provided on an isolated polynucleotide comprising at least 10 nucleotides of SEQ ID NO:2 that include alternative forms of the polymorphic sequences shown in Table 9.

For example, an isolated CRF2-13 polymorphic sequence can include from nucleotide 30957 to nucleotide 30967 of SEQ ID NO:3, provided that position 30962 is “A or “G”. In a second example, the isolated CRF2-13 polymorphic sequence can include at least 10 contiguous nucleotides from nucleotide 30650 to nucleotide 30660 of SEQ ID NO:3, provided that position 30655 is “A” or “G”. In additional examples, the isolated CRF2-13 nucleic acid sequence includes at least 10 contiguous nucleotides from nucleotide 28739 to nucleotide 28749 of SEQ ID NO:3, wherein position 28744 is “A” or “G”; at least 10 contiguous nucleotides from nucleotide 28442 to 28452 of SEQ ID NO:3, wherein position 28448 is “C” or “T”; additional examples include an isolated polynucleotide comprising at least 10 contiguous nucleotides from nucleotide 9421 to 9431 of SEQ ID NO:3, wherein position 9426 of the polynucleotide is “A” or “G”, or an isolated polynucleotide comprising at least 10 contiguous nucleotides from nucleotide 8806 to 8816 of SEQ ID NO:3, wherein position 8811 of the polynucleotide is “C or “T”.

Alternatively, an isolated CRF2-13 polymorphic sequence can include from nucleotide 32954 to nucleotide 32964 of SEQ ID NO:22, provided that position 30962 is “C” or “A”. Alternatively, the polymorphic sequence can include from nucleotide 31262 to 31272 of SEQ ID NO:22, provided that position 31266 is “C” or “T”; or nucleotides 30955 to 20965 of SEQ ID NO:22, provided that nucleotide 30960 is “T” or “C”; or nucleotides 29043 to 29053 of SEQ ID NO:22, provided that nucleotide 29048 is “C” or “T”; or nucleotides 28748 to 28758 of SEQ ID NO:22, provided that nucleotide 28753 is “G” or “A”; or nucleotides 23825 to 23835 of SEQ ID NO:22, provided that nucleotide 23830 is “G” or “A”.

In additional embodiments, the polymorphic nucleic acid includes at least 15, 20, 25, 50, 75, 100, 150, 250, 500, 750, or 1000 or more contiguous nucleotides from SEQ ID NO:3. In some embodiments, the polymorphic nucleotide sequence is 10-1000 nucleotides in length. For example, the polymorphic nucleotide sequence can be 20-750 nucleotides, 50-625 nucleotides, 75-500 nucleotides, 100-250 nucleotides in length.

Individuals carrying polymorphic alleles of the invention may be detected at either the DNA, the RNA, or the protein level using a variety of techniques that are well known in the art. Strategies for identification and detection are described in e.g., EP 730,663, EP 717,113, and PCT US97/02102. The present methods usually employ pre-characterized polymorphisms. That is, the genotyping location and nature of polymorphic forms present at a site have already been determined. The availability of this information allows sets of probes to be designed for specific identification of the known polymorphic forms.

Many of the methods described below require amplification of DNA from target samples. This can be accomplished by e.g., PCR. (1989), B. for detecting polymorphisms. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

The genomic DNA used for the diagnosis may be obtained from any nucleated cells of the body, such as those present in peripheral blood, urine, saliva, buccal samples, surgical specimen, and autopsy specimens. The DNA may be used directly or may be amplified enzymatically in vitro through use of PCR (Saiki et al. Science 239:487-491 (1988)) or other in vitro amplification methods such as the ligase chain reaction (LCR) (Wu and Wallace Genomics 4:560-569 (1989)), strand displacement amplification (SDA) (Walker et al. Proc. Natl. Acad. Sci. U.S.A, 89:392-396 (1992)), self-sustained sequence replication (3SR) (Fahy et al. PCR Methods P&J& 1:25-33 (1992)), prior to mutation analysis.

The detection of polymorphisms in specific DNA sequences, can be accomplished by a variety of methods including, but not limited to, restriction-fragment-length-polymorphism detection based on allele-specific restriction-endonuclease cleavage (Kan and Dozy Lancet ii:910-912 (1978)), hybridization with allele-specific oligonucleotide probes (Wallace et al. Nucl. Acids Res. 6:3543-3557 (1978)), including immobilized oligonucleotides (Saiki et al. Proc. Natl. Acad. SCI. USA, 86:6230-6234 (1969)) or oligonucleotide arrays (Maskos and Southern Nucl. Acids Res 21:2269-2270 (1993)), allele-specific PCR (Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Natl. Acad. Sci. U.S.A. 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. Proc. Natl. w Sci. U.S.A, 8Z4397-4401 (1988)) or enzymatic (Youil et al. Proc. Natl. Acad. Sci. U.S.A. 92:87-91 (1995)) cleavage of heteroduplex DNA, methods based on allele specific primer_extension (Syvanen et al. Genomics 8:684-692 (1990)), genetic bit analysis (GBA) (Nikiforov et al. &&I Acids 22:4167-4175 (1994)), the oligonucleotide-ligation assay (OLA) (Landegren et al. Science 241:1077 (1988)), the allele-specific ligation chain reaction (LCR) (Barrany Proc. Natl. Acad. Sci. U.S.A. 88:189-193 (1991)), gap-LCR (Abravaya et al. Nucl Acids Res 23:675-682 (1995)), radioactive and/or fluorescent DNA sequencing using standard procedures well known in the art, and peptide nucleic acid (PNA) assays (Orum et al., Nucl. Acids Res, 21:5332-5356 (1993); Thiede et al., Nucl. Acids Res. 24:983-984 (1996)).

CRF2-13 Variants

The invention further encompasses nucleic acid molecules that differ from the nucleotide sequences shown in SEQ ID NO:1 due to the degeneracy of the genetic code. These nucleic acids thus encode the same CRF2-13 protein as that encoded by the nucleotide sequence shown in SEQ ID NO:1, e.g., the polypeptide of SEQ ID NO:2. In another embodiment, an isolated nucleic acid molecule of the invention has a nucleotide sequence encoding a protein having an amino acid sequence shown in SEQ ID NO:2.

In addition to the human CRF2-13 nucleotide sequence shown in SEQ ID NO:1, it will be appreciated by those skilled in the art that DNA sequence polymorphisms that lead to changes in the amino acid sequences of CRF2-13 may exist within a population (e.g., the human population). Such genetic polymorphism in the CRF2-13 gene may exist among individuals within a population due to natural allelic variation. As used herein, the terms “gene” and “recombinant gene” refer to nucleic acid molecules comprising an open reading frame encoding a CRF2-13 protein, preferably a mammalian CRF2-13 protein. Such natural allelic variations can typically result in 1-5% variance in the nucleotide sequence of the CRF2-13 gene. Any and all such nucleotide variations and resulting amino acid polymorphisms in CRF2-13 that are the result of natural allelic variation and that do not alter the functional activity of CRF2-13 are intended to be within the scope of the invention.

Moreover, nucleic acid molecules encoding CRF2-13 proteins from other species, and thus that have a nucleotide sequence that differs from the human sequence of SEQ ID NO:1 are intended to be within the scope of the invention. Nucleic acid molecules corresponding to natural allelic variants and homologues of the CRF2-13 cDNAs of the invention can be isolated based on their homology to the human CRF2-13 nucleic acids disclosed herein using the human cDNAs, or a portion thereof, as a hybridization probe according to standard hybridization techniques under stringent hybridization conditions. For example, a soluble human CRF2-13 cDNA can be isolated based on its homology to human membrane-bound CRF2-13. Likewise, a membrane-bound human CRF2-13 cDNA can be isolated based on its homology to soluble human CRF2-13.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding CRF2-13 proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g, 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO:1 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO:1, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the CRF2-13 sequence that may exist in the population, the skilled artisan will further appreciate that changes can be introduced by mutation into the nucleotide sequence of SEQ ID NO:1, thereby leading to changes in the amino acid sequence of the encoded CRF2-13 protein, without altering the functional ability of the CRF2-13 protein. For example, nucleotide substitutions leading to amino acid substitutions at “non-essential” amino acid residues can be made in the sequence of SEQ ID NO:1. A “non-essential” amino acid residue is a residue that can be altered from the wild-type sequence of CRF2-13 without altering the biological activity, whereas an “essential” amino acid residue is required for biological activity. For example, amino acid residues that are conserved among the CRF2-13 proteins of the present invention, are predicted to be particularly unamenable to alteration.

Another aspect of the invention pertains to nucleic acid molecules encoding CRF2-13 proteins that contain changes in amino acid residues that are not essential for activity. Such CRF2-13 proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO:2. Preferably, the protein encoded by the nucleic acid is at least about 80% homologous to SEQ ID NO:2, more preferably at least about 90%, 95%, 98%, and most preferably at least about 99% homologous to SEQ ID NO:2.

An isolated nucleic acid molecule encoding a CRF2-13 protein homologous to the protein of SEQ ID NO:2 can be created by introducing one or more nucleotide substitutions, additions or deletions into the nucleotide sequence of SEQ ID NO:1, such that one or more amino acid substitutions, additions or deletions are introduced into the encoded protein.

Mutations can be introduced into the nucleotide sequence of SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in CRF2-13 is replaced with another amino acid residue from the same side chain family. Alternatively, in another embodiment, mutations can be introduced randomly along all or part of a CRF2-13 coding sequence, such as by saturation mutagenesis, and the resultant mutants can be screened for CRF2-13 biological activity to identify mutants that retain activity. Following mutagenesis of SEQ ID NO:1 the encoded protein can be expressed by any recombinant technology known in the art and the activity of the protein can be determined.

In one embodiment, a mutant CRF2-13 protein can be assayed for (1) the ability to form protein:protein interactions with other CRF2-13 proteins, other cell-surface proteins, or biologically active portions thereof, (2) complex formation between a mutant CRF2-13 protein and a CRF2-13 receptor; (3) the ability of a mutant CRF2-13 protein to bind to an intracellular target protein or biologically active portion thereof; (e.g., avidin proteins); (4) the ability to bind CRF2-13 protein; or (5) the ability to specifically bind an anti-CRF2-13 protein antibody.

Antisense CRF2-13 Nucleic Acids

Another aspect of the invention pertains to isolated antisense nucleic acid molecules that are hybridizable to or complementary to the nucleic acid molecule comprising the nucleotide sequence of SEQ ID NO:1, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire CRF2-13 coding strand, or to only a portion thereof. Nucleic acid molecules encoding fragments, homologs, derivatives and analogs of a CRF2-13 protein of SEQ ID NO:2, or antisense nucleic acids complementary to a CRF2-13 nucleic acid sequence of SEQ ID NO:1 are additionally provided.

In one embodiment, an antisense nucleic acid molecule is antisense to a “coding region” of the coding strand of a nucleotide sequence encoding CRF2-3. The term “coding region” refers to the region of the nucleotide sequence comprising codons which are translated into amino acid residues (e.g., the protein coding region of human CRF2-13 corresponds to SEQ ID NO:2). In another embodiment, the antisense nucleic acid molecule is antisense to a “noncoding region” of the coding strand of a nucleotide sequence encoding CRF2-13. The term “noncoding region” refers to 5′ and 3′ sequences which flank the coding region that are not translated into amino acids (i.e., also referred to as 5′ and 3′ untranslated regions).

Given the coding strand sequences encoding CRF2-13 disclosed herein (e.g., SEQ ID NO:1), antisense nucleic acids of the invention can be designed according to the rules of Watson and Crick or Hoogsteen base pairing. The antisense nucleic acid molecule can be complementary to the entire coding region of CRF2-13 mRNA, but more preferably is an oligonucleotide that is antisense to only a portion of the coding or noncoding region of CRF2-13 mRNA. For example, the antisense oligonucleotide can be complementary to the region surrounding the translation start site of CRF2-13 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CRF2-13 protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA—DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

CRF2-13 Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave CRF2-13 mRNA transcripts to thereby inhibit translation of CRF2-13 mRNA. A ribozyme having specificity for a CRF2-13-encoding nucleic acid can be designed based upon the nucleotide sequence of a CRF2-13 DNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a CRF2-13-encoding mRNA. See, e.g., Cech et al. U.S. Pat. No. 4,987,071; and Cech et al. U.S. Pat. No. 5,116,742. Alternatively, CRF2-13 mRNA can be used to select a catalytic RNA having a specific ribonuclease activity from a pool of RNA molecules. See, e.g., Bartel et al., (1993) Science 261:1411-1418.

Alternatively, CRF2-13 gene expression can be inhibited by targeting nucleotide sequences complementary to the regulatory region of the CRF2-13 (e.g., the CRF2-13 promoter and/or enhancers) to form triple helical structures that prevent transcription of the CRF2-13 gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of CRF2-13 can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of CRF2-13 can be used in therapeutic and diagnostic applications. For example, PNAs can be used as antisense or antigene agents for sequence-specific modulation of gene expression by, e.g., inducing transcription or translation arrest or inhibiting replication. PNAs of CRF2-13 can also be used, e.g., in the analysis of single base pair mutations in a gene by, e.g., PNA directed PCR clamping; as artificial restriction enzymes when used in combination with other enzymes, e.g, S1 nucleases (Hyrup B. (1996) above); or as probes or primers for DNA sequence and hybridization (Hyrup et al. (1996), above; Perry-O'Keefe (1996), above).

In another embodiment, PNAs of CRF2-13 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of CRF2-13 can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

CRF2-13 Polypeptides

A CRF2-13 polypeptide of the invention includes the CRF2-13-like protein whose sequence is provided in SEQ ID NO:2. In some embodiments, a CRF2-13 polypeptide includes amino acid sequences 21-520, amino acids 21-230 of SEQ ID NO:2, amino acids 21-246 of SEQ ID NO:2, amino acids 231-520 of SEQ ID NO:2, amino acids 247-520 of SEQ ID NO:2. The invention also includes a mutant or variant form of the disclosed CRF2-13 polypeptide, or of any of the fragments of the herein disclosed CRF2-13 polypeptide sequences.

Thus, a CRF2-13 polypeptide includes one in which any residues may be changed from the corresponding residue shown in SEQ ID NO:2 while still encoding a protein that maintains its CRF2-13-like activities and physiological functions, or a functional fragment thereof. In some embodiments, up to 20% or more of the residues may be so changed in the mutant or variant protein. In some embodiments, the CRF2-13 polypeptide according to the invention is a mature polypeptide.

In general, a CRF2-13-like variant that preserves CRF2-13-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated CRF2-13 proteins, and biologically active portions thereof, or derivatives, fragments, analogs or homologs thereof. Also provided are polypeptide fragments suitable for use as immunogens to raise anti-CRF2-13 antibodies. In one embodiment, native CRF2-13 proteins can be isolated from cells or tissue sources by an appropriate purification scheme using standard protein purification techniques. In another embodiment, CRF2-13 proteins are produced by recombinant DNA techniques. Alternative to recombinant expression, a CRF2-13 protein or polypeptide can be synthesized chemically using standard peptide synthesis techniques.

A “purified” protein or biologically active portion thereof is substantially free of cellular material or other contaminating proteins from the cell or tissue source from which the CRF2-13 protein is derived, or substantially free from chemical precursors or other chemicals when chemically synthesized. The language “substantially free of cellular material” includes preparations of CRF2-13 protein in which the protein is separated from cellular components of the cells from which it is isolated or recombinantly produced. In one embodiment, the language “substantially free of cellular material” includes preparations of CRF2-13 protein having less than about 30% (by dry weight) of non-CRF2-13 protein (also referred to herein as a “contaminating protein”), more preferably less than about 20% of non-CRF2-13 protein, still more preferably less than about 10% of non-CRF2-13 protein, and most preferably less than about 5% non-CRF2-13 protein. When the CRF2-13 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CRF2-13 protein in which the protein is separated from chemical precursors or other chemicals that are involved in the synthesis of the protein. In one embodiment, the language “substantially free of chemical precursors or other chemicals” includes preparations of CRF2-13 protein having less than about 30% (by dry weight) of chemical precursors or non-CRF2-13 chemicals, more preferably less than about 20% chemical precursors or non-CRF2-13 chemicals, still more preferably less than about 10% chemical precursors or non-CRF2-13 chemicals, and most preferably less than about 5% chemical precursors or non-CRF2-13 chemicals.

Biologically active portions of a CRF2-13 protein include peptides comprising amino acid sequences sufficiently homologous to or derived from the amino acid sequence of the CRF2-13 protein, e.g., the amino acid sequence shown in SEQ ID NO:2 that include fewer amino acids than the full length CRF2-13 proteins, and exhibit at least one activity of a CRF2-13 protein. Typically, biologically active portions comprise a domain or motif with at least one activity of the CRF2-13 protein. A biologically active portion of a CRF2-13 protein can be a polypeptide which is, for example, 10, 25, 50, 100 or more amino acids in length.

A biologically active portion of a CRF2-13 protein of the present invention may contain at least one of the above-identified domains conserved between the CRF2-13 proteins. Moreover, other biologically active portions, in which other regions of the protein are deleted, can be prepared by recombinant techniques and evaluated for one or more of the functional activities of a native CRF2-13 protein.

In an embodiment, the CRF2-13 protein has an amino acid sequence shown in SEQ ID NO:2. In other embodiments, the CRF2-13 protein is substantially homologous to SEQ ID NO:2 and retains the functional activity of the protein of SEQ ID NO:2, yet differs in amino acid sequence due to natural allelic variation or mutagenesis, as described in detail below. Accordingly, in another embodiment, the CRF2-13 protein is a protein that comprises an amino acid sequence at least about 45% homologous to the amino acid sequence of SEQ ID NO:2 and retains the functional activity of the CRF2-13 proteins of SEQ ID NO:2.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides CRF2-13 chimeric or fusion proteins. As used herein, a CRF2-13 “chimeric protein” or “fusion protein” comprises a CRF2-13 polypeptide operatively linked to a non-CRF2-13 polypeptide. An “CRF2-13 polypeptide” refers to a polypeptide having an amino acid sequence corresponding to CRF2-13, whereas a “non-CRF2-13 polypeptide” refers to a polypeptide having an amino acid sequence corresponding to a protein that is not substantially homologous to the CRF2-13 protein, e.g., a protein that is different from the CRF2-13 protein and that is derived from the same or a different organism. Within a CRF2-13 fusion protein the CRF2-13 polypeptide can correspond to all or a portion of a CRF2-13 protein. An example of a CRF2-13 fusion polypeptide is one that includes amino acids 21-230 of SEQ ID NO:2 (e.g., a polypeptide that includes amino acids 1-246 or amino acids 21-246 of SEQ ID NO:2). In one embodiment, a CRF2-13 fusion protein comprises at least one biologically active portion of a CRF2-13 protein. In another embodiment, a CRF2-13 fusion protein comprises at least two biologically active portions of a CRF2-13 protein. Within the fusion protein, the term “operatively linked” is intended to indicate that the CRF2-13 polypeptide and the non-CRF2-13 polypeptide are fused in-frame to each other. The non-CRF2-13 polypeptide can be fused to the N-terminus or C-terminus of the CRF2-13 polypeptide.

For example, in one embodiment a CRF2-13 fusion protein comprises a CRF2-13 polypeptide operably linked to either an extracellular domain of a second protein, i.e., non-CRF2-13 protein, or to the transmembrane and intracellular domain of a second protein, i.e., non-CRF2-13 protein. Such fusion proteins can be further utilized in screening assays for compounds that modulate CRF2-13 activity (such assays are described in detail below).

In another embodiment, the fusion protein is a GST-CRF2-13 fusion protein in which the CRF2-13 sequences are fused to the C-terminus of the GST (i.e., glutathione S-transferase) sequences. Such fusion proteins can facilitate the purification of recombinant CRF2-13.

In another embodiment, the fusion protein is a CRF2-13-immunoglobulin fusion protein in which the CRF2-13 sequences comprising one or more domains are fused to sequences derived from a member of the immunoglobulin protein family.

The CFR2-13 fusion proteins (e.g., CRF2-13-immunoglobulin fusion proteins) of the invention can be incorporated into pharmaceutical compositions and administered to a subject to inhibit or augment an interaction between a cell surface receptor and its ligand. This could occur either by 1) binding to and removing available ligand for the receptor (Fe mediated scavenging of the ligand affecting bioavailability); 2) binding to the ligand and blocking its ability to bind to the cell receptor (antagonizing or neutralizing); 3) associating with another CRF member and thereby modulating (e.g., inhibiting) a downstream signal transduction cascade; 4) associating with either a ligand or another CRF and facilitating the activity of the ligand. By all of these mechanisms, a CRF2-13 protein may be used to modulate the interaction between a CRF2 receptor and its cognate ligand (e.g., an interaction between IL-10 and an IL-10 receptor and interaction between IL-22 and an IL-22 receptor).

Inhibition of the CRF2-13 ligand/CRF2-13 interaction can be used therapeutically for both the treatment of proliferative and differentiative disorders, e,g., cancer, modulating (e.g., promoting or inhibiting) cell survival as well as immunomodulatory disorders, autoimmunity, transplantation, and inflammation by alteration of cyotokine and chemokine cascade mechanisms. Moreover, the CRF2-13-immunoglobulin fusion proteins of the invention can be used as immunogens to produce anti-CRF2-13 antibodies in a subject, to purify CRF2-13 ligands, and in screening assays to identify molecules that inhibit the interaction of CRF2-13 with a CRF2-13 ligand.

A CRF2-13 chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A CRF2-13-encoding nucleic acid can be cloned into such an expression vector such that the fusion moiety is linked in-frame to the CRF2-13 protein.

Polypeptide Libraries

In addition, libraries of fragments of the CRF2-13 protein coding sequence can be used to generate a variegated population of CRF2-13 fragments for screening and subsequent selection of variants of a CRF2-13 protein. In one embodiment, a library of coding sequence fragments can be generated by treating a double stranded PCR fragment of a CRF2-13 coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the CRF2-13 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CRF2-13 proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify CRF2-13 variants (Arkin and Yourvan (1992) PNAS 89:7811-7815; Delgrave et al. (1993) Protein Engineering 6:327-331).

CRF2-13 Antibodies

Also included in the invention are antibodies to CRF2-13 proteins, or fragments of CRF2-13 proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated CRF2-13-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO:2, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of CRF2-13-related protein that is located on the surface of the protein, e.g., a hydrophilic region. A hydrophobicity analysis of the human CRF2-13-related protein sequence will indicate which regions of a CRF2-13-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F(ab′)₂ fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi ¹³¹I, ¹¹³In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

CRF2-13 Recombinant Expression Vectors and Host Cells

Another aspect of the invention pertains to vectors, preferably expression vectors, containing a nucleic acid encoding a CRF2-13 protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CRF2-13 proteins, mutant forms of CRF2-13 proteins, fusion proteins, etc.).

The recombinant expression vectors of the invention can be designed for expression of CRF2-13 proteins in prokaryotic or eukaryotic cells. For example, CRF2-13 proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the CRF2-13 expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, CRF2-13 can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to CRF2-13 mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, CRF2-13 protein can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding CRF2-13 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) CRF2-13 protein. Accordingly, the invention further provides methods for producing CRF2-13 protein using the host cells of the invention. In one embodiment, the method comprises culturing the host cell of invention (into which a recombinant expression vector encoding CRF2-13 protein has been introduced) in a suitable medium such that CRF2-13 protein is produced. In another embodiment, the method further comprises isolating CRF2-13 protein from the medium or the host cell.

Transgenic CRF2-13 Animals

The host cells of the invention can also be used to produce non-human transgenic animals. For example, in one embodiment, a host cell of the invention is a fertilized oocyte or an embryonic stem cell into which CRF2-13 protein-coding sequences have been introduced. Such host cells can then be used to create non-human transgenic animals in which exogenous CRF2-13 sequences have been introduced into their genome or homologous recombinant animals in which endogenous CRF2-13 sequences have been altered. Such animals are useful for studying the function and/or activity of CRF2-13 protein and for identifying and/or evaluating modulators of CRF2-13 protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous CRF2-13 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing CRF2-13-encoding nucleic acid into the male pronuclei of a fertilized oocyte (e.g., by microinjection, retroviral infection) and allowing the oocyte to develop in a pseudopregnant female foster animal. Sequences including SEQ ID NO:1 can be introduced as a transgene into the genome of a non-human animal. Alternatively, a non-human homologue of the human CRF2-13 gene, such as a mouse CRF2-13 gene, can be isolated based on hybridization to the human CRF2-13 cDNA (described further supra) and used as a transgene. Intronic sequences and polyadenylation signals can also be included in the transgene to increase the efficiency of expression of the transgene. A tissue-specific regulatory sequence(s) can be operably-linked to the CRF2-13 transgene to direct expression of CRF2-13 protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the CRF2-13 transgene in its genome and/or expression of CRF2-13 mRNA in tissues or cells of the animals. A transgenic founder animal can then be used to breed additional animals carrying the transgene. Moreover, transgenic animals carrying a transgene-encoding CRF2-13 protein can further be bred to other transgenic animals carrying other transgenes.

To create a homologous recombinant animal, a vector is prepared which contains at least a portion of a CRF2-13 gene into which a deletion, addition or substitution has been introduced to thereby alter, e.g., functionally disrupt, the CRF2-13 gene. The CRF2-13 gene can be a human gene (e.g., the DNA of SEQ ID NO:1), but more preferably, is a non-human homologue of a human CRF2-13 gene. For example, a mouse homologue of human CRF2-13 gene of SEQ ID NO:1 can be used to construct a homologous recombination vector suitable for altering an endogenous CRF2-13 gene in the mouse genome. In one embodiment, the vector is designed such that, upon homologous recombination, the endogenous CRF2-13 gene is functionally disrupted (i.e., no longer encodes a functional protein; also referred to as a “knock out” vector).

Alternatively, the vector can be designed such that, upon homologous recombination, the endogenous CRF2-13 gene is mutated or otherwise altered but still encodes functional protein (e.g., the upstream regulatory region can be altered to thereby alter the expression of the endogenous CRF2-13 protein). In the homologous recombination vector, the altered portion of the CRF2-13 gene is flanked at its 5′- and 3′-termini by additional nucleic acid of the CRF2-13 gene to allow for homologous recombination to occur between the exogenous CRF2-13 gene carried by the vector and an endogenous CRF2-13 gene in an embryonic stem cell. The additional flanking CRF2-13 nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced CRF2-13 gene has homologously-recombined with the endogenous CRF2-13 gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The CRF2-13 nucleic acid molecules, CRF2-13 proteins, and anti-CRF2-13 antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a CRF2-13 protein or anti-CRF2-13 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., 1993 Proc. Natl. Acad. Sci. USA, 90: 7889-7893. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Screening and Detection Methods

The isolated nucleic acid molecules of the invention can be used to express CRF2-13 protein (e.g., via a recombinant expression vector in a host cell in gene therapy applications), to detect CRF2-13 mRNA (e.g., in a biological sample) or a genetic lesion in a CRF2-13 gene, and to modulate CRF2-13 activity, as described further, below. In addition, the CRF2-13 proteins can be used to screen drugs or compounds that modulate the CRF2-13 protein activity or expression as well as to treat disorders characterized by insufficient or excessive production of CRF2-13 protein or production of CRF2-13 protein forms that have decreased or aberrant activity compared to CRF2-13 wild-type protein. In addition, the anti-CRF2-13 antibodies of the invention can be used to detect and isolate CRF2-13 proteins and modulate CRF2-13 activity. For example, CRF2-13 activity includes T-cell or NK cell growth and differentiation, antibody production, and tumor growth.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to CRF2-13 proteins or have a stimulatory or inhibitory effect on, e.g., CRF2-13 protein expression or CRF2-13 protein activity. The invention also includes compounds identified in the screening assays described herein.

In one embodiment, the invention provides assays for screening candidate or test compounds which bind to or modulate the activity of the membrane-bound form of a CRF2-13 protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of CRF2-13 protein, or a biologically-active portion thereof, on the cell surface is contacted with a test compound and the ability of the test compound to bind to a CRF2-13 protein determined. The cell, for example, can be of mammalian origin or a yeast cell. Determining the ability of the test compound to bind to the CRF2-13 protein can be accomplished, for example, by coupling the test compound with a radioisotope or enzymatic label such that binding of the test compound to the CRF2-13 protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of CRF2-13 protein, or a biologically-active portion thereof, on the cell surface with a known compound which binds CRF2-13 to form an assay mixture, contacting the assay mixture with a test compound, and determining the ability of the test compound to interact with a CRF2-13 protein, wherein determining the ability of the test compound to interact with a CRF2-13 protein comprises determining the ability of the test compound to preferentially bind to CRF2-13 protein or a biologically-active portion thereof as compared to the known compound.

In another embodiment, an assay is a cell-based assay comprising contacting a cell expressing a membrane-bound form of CRF2-13 protein, or a biologically-active portion thereof, on the cell surface with a test compound and determining the ability of the test compound to modulate (e.g., stimulate or inhibit) the activity of the CRF2-13 protein or biologically-active portion thereof. Determining the ability of the test compound to modulate the activity of CRF2-13 or a biologically-active portion thereof can be accomplished, for example, by determining the ability of the CRF2-13 protein to bind to or interact with a CRF2-13 target molecule. As used herein, a “target molecule” is a molecule with which a CRF2-13 protein binds or interacts in nature, for example, a molecule on the surface of a cell which expresses a CRF2-13 interacting protein, a molecule on the surface of a second cell, a molecule in the extracellular milieu, a molecule associated with the internal surface of a cell membrane or a cytoplasmic molecule. A CRF2-13 target molecule can be a non-CRF2-13 molecule or a CRF2-13 protein or polypeptide of the invention In one embodiment, a CRF2-13 target molecule is a component of a signal transduction pathway that facilitates transduction of an extracellular signal (e.g. a signal generated by binding of a compound to a membrane-bound CRF2-13 molecule) through the cell membrane and into the cell. The target, for example, can be a second intercellular protein that has catalytic activity or a protein that facilitates the association of downstream signaling molecules with CRF2-13.

Determining the ability of the CRF2-13 protein to bind to or interact with a CRF2-13 target molecule can be accomplished by one of the methods described above for determining direct binding. In one embodiment, determining the ability of the CRF2-13 protein to bind to or interact with a CRF2-13 target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a CRF2-13-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a CRF2-13 protein or biologically-active portion thereof with a test compound and determining the ability of the test compound to bind to the CRF2-13 protein or biologically-active portion thereof. Binding of the test compound to the CRF2-13 protein can be determined either directly or indirectly as described above. In one such embodiment, the assay comprises contacting the CRF2-13 protein or biologically-active portion thereof with a known compound which binds CRF2-13 to form an assay mixture, contacting the assay mixture with a test compound, and determining the ability of the test compound to interact with a CRF2-13 protein, wherein determining the ability of the test compound to interact with a CRF2-13 protein comprises determining the ability of the test compound to preferentially bind to CRF2-13 or biologically-active portion thereof as compared to the known compound.

In still another embodiment, an assay is a cell-free assay comprising contacting CRF2-13 protein or biologically-active portion thereof with a test compound and determining the ability of the test compound to modulate (e.g. stimulate or inhibit) the activity of the CRF2-13 protein or biologically-active portion thereof. Determining the ability of the test compound to modulate the activity of CRF2-13 can be accomplished, for example, by determining the ability of the CRF2-13 protein to bind to a CRF2-13 target molecule by one of the methods described above for determining direct binding. In an alternative embodiment, determining the ability of the test compound to modulate the activity of CRF2-13 protein can be accomplished by determining the ability of the CRF2-13 protein further modulate a CRF2-13 target molecule. For example, the catalytic/enzymatic activity of the target molecule on an appropriate substrate can be determined as described above.

In yet another embodiment, the cell-free assay comprises contacting the CRF2-13 protein or biologically-active portion thereof with a known compound which binds CRF2-13 protein to form an assay mixture, contacting the assay mixture with a test compound, and determining the ability of the test compound to interact with a CRF2-13 protein, wherein determining the ability of the test compound to interact with a CRF2-13 protein comprises determining the ability of the CRF2-13 protein to preferentially bind to or modulate the activity of a CRF2-13 target molecule.

The cell-free assays of the invention are amenable to use of both the soluble form or the membrane-bound form of CRF2-13 protein. In the case of cell-free assays comprising the membrane-bound form of CRF2-13 protein, it may be desirable to utilize a solubilizing agent such that the membrane-bound form of CRF2-13 protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either CRF2-13 protein or its target molecule to facilitate separation of complexed from uncomplexed forms of one or both of the proteins, as well as to accommodate automation of the assay. Binding of a test compound to CRF2-13 protein, or interaction of CRF2-13 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-CRF2-13 fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or CRF2-13 protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of CRF2-13 protein binding or activity determined using standard techniques.

Other techniques for immobilizing proteins on matrices can also be used in the screening assays of the invention. For example, either the CRF2-13 protein or its target molecule can be immobilized utilizing conjugation of biotin and streptavidin. Biotinylated CRF2-13 protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with CRF2-13 protein or target molecules, but which do not interfere with binding of the CRF2-13 protein to its target molecule, can be derivatized to the wells of the plate, and unbound target or CRF2-13 protein trapped in the wells by antibody conjugation. Methods for detecting such complexes, in addition to those described above for the GST-immobilized complexes, include immunodetection of complexes using antibodies reactive with the CRF2-13 protein or target molecule, as well as enzyme-linked assays that rely on detecting an enzymatic activity associated with the CRF2-13 protein or target molecule.

In another embodiment, modulators of CRF2-13 protein expression are identified in a method wherein a cell is contacted with a candidate compound and the expression of CRF2-13 mRNA or protein in the cell is determined. The level of expression of CRF2-13 mRNA or protein in the presence of the candidate compound is compared to the level of expression of CRF2-13 mRNA or protein in the absence of the candidate compound. The candidate compound can then be identified as a modulator of CRF2-13 mRNA or protein expression based upon this comparison. For example, when expression of CRF2-13 mRNA or protein is greater (i.e., statistically significantly greater) in the presence of the candidate compound than in its absence, the candidate compound is identified as a stimulator of CRF2-13 mRNA or protein expression. Alternatively, when expression of CRF2-13 mRNA or protein is less (statistically significantly less) in the presence of the candidate compound than in its absence, the candidate compound is identified as an inhibitor of CRF2-13 mRNA or protein expression. The level of CRF2-13 mRNA or protein expression in the cells can be determined by methods described herein for detecting CRF2-13 mRNA or protein.

In yet another aspect of the invention, the CRF2-13 proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with CRF2-13 (“CRF2-13-binding proteins” or “CRF2-13-bp”) and modulate CRF2-13 activity. Such CRF2-13-binding proteins are also likely to be involved in the propagation of signals by the CRF2-13 proteins as, for example, upstream or downstream elements of the CRF2-13 pathway.

The two-hybrid system is based on the modular nature of most transcription factors, which consist of separable DNA-binding and activation domains. Briefly, the assay utilizes two different DNA constructs. In one construct, the gene that codes for CRF2-13 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CRF2-13-dependenht complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with CRF2-13.

The invention further pertains to novel agents identified by the aforementioned screening assays and uses thereof for treatments as described herein.

The invention will be further illustrated in the following non-limiting examples.

### EXAMPLE 1

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:4. The variant amino acid sequence is shown in bold-font. A valine at position 30 in the polypeptide sequence shown in SEQ ID NO:2 is replaced with an alanine in SEQ ID NO:4.

(SEQ ID NO:4)
MAGPERWGPLLLCLLQAAPGRPRLAPPQNATLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWOTLPPEPNLVPGGPPVS
LQTLTFCWESSPEEEEEARESEIEDSDAGSWGAESTQRTEDRGRTLGHYMAR

### EXAMPLE 2

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:5. The variant amino acid sequence is shown in bold-font. A leucine at position 39 in the polypeptide sequence shown in SEQ ID NO:2 is replaced with an isoleucine in SEQ ID NO:5.

(SEQ ID NO:5)
MAGPERWGPLLLCLLQAAPGRPRLAPPQNVTLLSQNFSVYITWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEOAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 3

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:6. The variant amino acid sequence is shown in bold-font. An asparagine at position 49 in the polypeptide sequence shown in SEQ ID NO:2 is replaced with a threonine in SEQ ID NO:6.

(SEQ ID NO:5)
MAGPERWGPLLLCLLQAAPGRPRLAPPQNVTLLSQNFSVYLTWLPGLGTPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 4

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:7. The variant amino acid sequence is shown in bold-font. An arginine at position 65 in the polypeptide sequence shown in SEQ ID NO:2 is replaced with a lysine in SEQ ID NO:7.

(SEQ ID NO:7)
MAGPERWGPLLLCLLQAAPGRPRLAPPQNVTLLSQNFSVYLTWLPGLGNPQDVTYFVAYQSSPTKRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 5

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:8. The variant amino acid sequence is shown in bold-font. A lysine at position 78 in the polypeptide sequence shown in SEQ ID NO:2 is replaced with an arginine in SEQ ID NO:8.

(SEQ ID NO:8)
MAGPERWGPLLLCLLQAAPGRPRLAPPQNVTLLSQNFSVYLTWLPGLGNPQDVTYFVAYQSSPTRRRWREVEECAGTR
]
ELLCSMMCLKKQDLYNKFKGRVRTVSPSSKSPWVESEYLDYLFEVEPAPPVLVLTQTEEILSANATYQLPPCMPPLDL
]
KYEVAFWKEGAGNKTLFPVTPHGQPVQITLQPAASEHHCLSARTIYTFSVPKYSKFSKPTCFLLEVPEANWAFLVLPS
]
LLILLLVIAAGGVIWKTLMGNPWFQRAKMPRALDFSGHTHPVATFQPSRPESVNDLFLCPQKELTRGVRPTPRVRAPA
]
TQQTRWKKDLAEDEEEEDEEDTEDGVSFQPYIEPPSFLGQEHQAPGHSEAGGVDSGRPRAPLVPSEGSSAWDSSDRSW
]
ASTVDSSWDRAGSSGYLAEKGPGQGPGGDGHQESLPPPEFSKDSGFLEELPEDNLSSWATWGTLPPEPNLVPGGPPVS
]
LQTLTFCWESSPEEEEEARESEIEDSDAGSWGAESTQRTEDRGRTLGHYMAR

### EXAMPLE 6

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:9. The variant amino acid sequence is shown in bold-font. A Q {glutamine?}at position 90 in the polypeptide sequence shown in SEQ ID NO:2 is replaced with an asparagine in SEQ ID NO:9.

(SEQ ID NO:9)
MAGPERWGPLLLCLLQAAPGRPRLAPPQNVTLLSQNFSVYLTWLPGLGNPQDVTYFVAYQSSPTRRRWREVEECAGTK
ELLCSMMCLKKNDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 7

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:10. The variant amino acid sequence is shown in bold-font. A arginine at position 99 in the polypeptide sequence shown in SEQ ID NO:2 is replaced with an lysine in SEQ ID NO:10.

(SEQ ID NO:10)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGKVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLOQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 8

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:11. The variant amino acid sequence is shown in bold-font. A valine at position 112 in the polypeptide sequence shown in SEQ ID NO:2 is replaced with an leucine in SEQ ID NO:.11.

(SEQ ID NO:11)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWLESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 9

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:12. The variant amino acid sequence is shown in bold-font. A tyrosine at position 119 in the polypeptide sequence shown in SEQ ID NO:2 is replaced with a phenylalanine in SEQ ID NO:12.

(SEQ ID NO:12)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWLESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### Example 10

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:13. The variant amino acid sequence is shown in bold-font. A valine at position 129 in the polypeptide sequence shown in SEQ ID NO:2 is replaced with an isoleucine in SEQ ID NO:13.

(SEQ ID NO:13)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPILVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSOHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSORPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 11

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:14. The variant amino acid sequence is shown in bold-font. A threonine at position 144 in the polypeptide sequence shown in SEQ ID NO:2 is replaced with an asparagine in SEQ ID NO:14.

(SEQ ID NO:14)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANANYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 12

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:15. The variant amino acid sequence is shown in bold-font. A leucine at position 154 in the polypeptide sequence shown in SEQ ID NO:2 is replaced with an alanine in SEQ ID NO:15.

(SEQ ID NO:15)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPADL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 13

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:16. The variant amino acid sequence is shown in bold-font. A lysine at position 170 in the polypeptide sequence shown in SEQ ID NO:2 is replaced with an arginine in SEQ ID NO:16.

(SEQ ID NO:16)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNRTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 14

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:17. The variant amino acid sequence is shown in bold-font. A valine at position 175 in the polypeptide sequence shown in SEQ ID NO:2 is replaced with a leucine in SEQ ID NO:17.

(SEQ ID NO:17)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPLTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### EXAMPLE 15

### A sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:18. The variant amino acid sequence is shown in bold-font. An alanine at position 189 in the polypeptide sequence shown in SEQ ID NO:2 is replaced with a valine in SEQ ID NO:18.

(SEQ ID NO:18)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPVASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPOHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWOAESTQRTEDRGRTLGHYMAR

### Example 16

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:19 The variant amino acid sequence is shown in bold-font. An arginine at position 199 in the polypeptide sequence shown in SEQ ID NO:2 is replaced with a lysine in SEQ ID NO:.19

| | |
|---|---|
| MAGPERWGPLLLCLLQAAPGRPRLAPPQNVTLLSQNFSVYLTWLPGLGNPQDVTYFVAYQSSPTRRRWREVEECAGTK | (SEQ ID NO:19) |
| ELLCSMMCLKKQDLYNKFKGRVRTVSPSSKSPWVESEYLDYLFEVEPAPPVLVLTQTEEILSANATYQLPPCMPPLDL | |
| KYEVAFWKEGAGNKTLFPVTPHGQPVQITLQPAASEHHCLSARTIYTFSVPKYSKWSKPTCFLLEVPEANWAFLVLPS | |
| LLILLLVIAAGGVIWKTLMGNPWFQRAKMPRALDFSGHTHPVATFQPSRPESVNDLFLCPQKELTRGVRPTPRVRAPA | |
| TQQTRWKKDLAEDEEEEDEEDTEDGVSFQPYIEPPSFLGQEHQAPGHSEAGGVDSGRPRAPLVPSEGSSAWDSSDRSW | |
| ASTVDSSWDRAGSSGYLAEKGPGQGPGGDGHQESLPPPEFSKDSGFLEELPEDNLSSWATWGTLPPEPNLVPGGPPVS | |
| LQTLTFCWESSPEEEEEARESEIEDSDAGSWGAESTQRTEDRGRTLGHYMAR | |

### EXAMPLE 17

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:20. The variant amino acid sequence is shown in bold-font. A phenylalanine at position 212 in the polypeptide sequence shown in SEQ ID NO:2 is replaced with an a tryptophan in SEQ ID NO:20.

(SEQ ID NO:20)
MAGPERWGPLLLCLLQAAPGRPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKWSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGHQESLPPPEFSKDSGFLEELPEDNLSSWATWGTLPPEPNLVPGGPPVS
LQTLTFCWESSPEEEEEARESEIEDSDAGSWGAESTQRTEDRGRTLGHYMAR

### Example 18

### A Sequence Variant of the Disclosed CRF2-13 Polypeptide Amino Acid Sequence (SEQ ID NO:2)

A polypeptide sequence differing by one amino acid sequence from the amino acid sequence of SEQ ID NO:2 is shown in SEQ ID NO:21. The variant amino acid sequence is shown in bold-font. An arginine at position 230 in the polypeptide sequence shown in SEQ ID NO:2 is replaced with a lysine in SEQ ID NO21:.

(SEQ ID NO:21)
MAGPERWGPLLLCLLQAAPORPRLAPPQNVTLLSQNFSVYLTWLPGLGNPQDVTYFVAYQSSPTRRRWREVEECAGTK
ELLCSMMCLKKQDLYNKFKGRVRTVSPSSKSPWVESEYLDYLFEVEPAPPVLVLTQTEEILSANATYQLPPCMPPLDL
KYEVAFWKEGAGNKTLFPVTPHGQPVQITLQPAASEHHCLSARTIYTFSVPKYSKFSKPTCFLLEVPEANWAFLVLPS
LLILLLVIAAGGVIWKTLMGNPWFQRAKMPRALDFSGHTHPVATFQPSRPESVNDLFLCPQKELTRGVRPTPRVRAPA
TQQTRWKKDLAEDEEEEDEEDTEDGVSFQPYIEPPSFLGQEHQAPGHSEAGGVDSGRPRAPLVPSEGSSAWDSSDRSW
ASTVDSSWDRAGSSGYLAEKGPGQGPGGDGEQESLPPPEFSKDSGFLEELPEDNLSSWATWGTLPPEPNLVPGGPPVS
LQTLTFCWESSPEEEEEARESEIEDSDACSWGAESTQRTEDRGRTLGHYMAK

### EXAMPLE 19

### Identification of a CRF2-13 Sequence in a Human Placental cDNA Library

A 310 nucleotide fragment corresponding to nucleotides XX to XX [41-352 of SEQ ID No.1] in Table 1 was identified in a human placental cDNA library (BD Biosciences Clontech, Palo Alto, Calif., USA) by PCR using an Advantage II PCR kit (BD Biosciences Clontech, Palo Alto, Calif., USA) and primers specific for the 5′ region of the human CRF2-13. The primers included Ax5-1 (GCTGCAGGCCGCTCCAGGGAGGCCCCG; SEQ ID:23) and Ax3-1 (CCAGGTATTCGGACTCCACCCAGGGGGAC; SEQ ID NO:24). The primers were used for thirty eight thermal cycles of PCR. The CRF2-13 nucleic acid product was gel purified and sequenced. The sequence corresponds to the corresponding sequences in the CRF2-13 sequence disclosed in Table 1.

Based on these findings a Rapid-Screen™ Arrayed cDNA Library Panel of Human Placenta Sub-Plate 2H (Origene Technologies, Inc., Rockville, Md., USA) was selected for screening and isolation of the CFR2-13 clone coding for the mature protein. [11-1563 of SEQ ID No.1]. The existence of the first 10 bases of SEQ ID No.1 was verified by PCR. The library quality was improved by first isolating double-stranded cDNAs of different size-fractions and then ligating them separately into the vector. The cDNA library is arrayed in a 96-well plates.

Since the cDNAs of the Human Placenta Sub-Plate 2H human placental library were directionally-cloned into the CMV expression vector pCMV6-XL4, a vector-derived 5′ PCR primer was used in conjunction with a gene-specific 3′ reverse primer to identify the CRF2-13 clone. In this study, the cDNA library was screened by a PCR-based procedure using the Advantage II PCR kit (BD Biosciences Clontech, Palo Alto, Calif., USA) and Ax5-1 (SEQ ID:25 and Ax3-2 (TTGGTTCCCGCACACTCTTCCACTTCG; SEQ ID NO:26) as PCR primers. PCR analysis was carried out in a 96-well arrayed at 50 clones per well. The PCR positive well (E2) was identified and the E. coli cells from that well were subsequently diluted, plated out and analyzed to yield the clone full-length CRF2-13 clone. The identity of the CRF2-13 clone was then verified by sequence analysis.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid molecule encoding a polypeptide comprising an amino acid sequence at least 95% identical to amino acids 21-520 of SEQ ID NO:2.

2. The isolated nucleic acid molecule of claim 1, wherein said nucleic acid molecule encodes a polypeptide comprising an amino acids 21-520 of SEQ ID NO:2.

3. The isolated nucleic acid molecule of claim 1, wherein said nucleic acid molecule encodes a polypeptide with an amino acid sequence having one or more substitutions relative to the amino acid sequence of amino acids 21-520 of SEQ ID NO:2.

4. The nucleic acid molecule of claim 9, wherein said molecule hybridizes under stringent conditions to a nucleic acid sequence complementary to a nucleic acid molecule comprising SEQ ID NO:1.

5. A vector comprising the nucleic acid molecule of claim 1 and a pharmaceutically acceptable carrier.

6. A cell containing the vector of claim 5.

7. A substantially purified polypeptide comprising an amino acid sequence at least 95% identical to the amino acid sequence of amino acids 21-520 of SEQ ID NO:2.

8. The substantially purified polypeptide of claim 7, wherein said polypeptide comprises amino acids 21-520 of SEQ ID NO:2.

9. A pharmaceutical composition comprising the polypeptide of claim 7 and a pharmaceutically acceptable carrier.

10. A fusion polypeptide comprising the polypeptide of claim 7 operably linked to a non-CRF2-13 polypeptide.

11. The fusion polypeptide of claim 10, wherein said non-CRF2-13 polypeptide is selected from the group consisting of an Fc region of an immunoglobulin molecules or a FLAG epitope, a HIS tag, and a MYC tag.

12. A pharmaceutical composition comprising the fusion polypeptide of claim 10 and a pharmaceutically acceptable carrier.

13. An antibody that binds selectively to the substantially purified polypeptide of claim 7.

14. The antibody of claim 13, wherein said antibody is a polyclonal antibody.

15. The antibody of claim 13, wherein said antibody is a monoclonal antibody.

16. The monoclonal antibody of claim 13, wherein said monoclonal antibody is selected from the group consisting of a murine monoclonal antibody, and a humanized monoclonal antibody.

17. A method of detecting the presence of a CRF2-13 nucleic acid molecule in a biological sample, the method comprising:
contacting the sample with a nucleic acid probe; and
identifying the bound probe, if present,
thereby detecting the presence of CRF2-13 nucleic acid molecule in said sample.

18. A method of detecting the presence of a CRF2-13 polypeptide in a sample, the method comprising:
contacting the sample with a compound that selectively binds to said polypeptide under conditions allowing for formation of a complex between said polypeptide and said compound; and
detecting said complex, if present, thereby identifying said polypeptide in said sample.

19. A method of modulating the activity of a CRF2-13 polypeptide, the method comprising contacting a cell sample comprising said polypeptide with a compound that binds to said polypeptide in an amount sufficient to modulate the activity of the polypeptide.

20. A method of treating or preventing a pathological condition associated with a cytokine-mediated disorder, the method comprising administering to a subject an agent that increases levels of a polypeptide comprising the extracellular amino acid sequence of a CRF2-13 polypeptide in an amount sufficient to alleviate or prevent the pathological condition in said subject.
